(19) United States
(12) Patent Application Publication
(10) Pub. No.: US 2025/0281585 A1
(43) Pub. Date: Sep. 11, 2025

(54) VETERINARY COMPOSITIONS OF MODIFIED VIRUS-LIKE PARTICLES OF CMV AND IL-1 BETA MUTEIN ANTIGENS

(71) Applicants: Elanco US Inc., Greenfield, IN (US); Saiba Animal Health AG, Pfäffikon (CH)

(72) Inventors: Andris ZELTINS, Pfäffikon (CH); Jonathan SNYDER, New Palestine, IN (US)

(21) Appl. No.: 19/070,668

(22) Filed: Mar. 5, 2025

Related U.S. Application Data
(60) Provisional application No. 63/562,153, filed on Mar. 6, 2024.

(51) Int. Cl. A61K 39/00; A61K 39/385; A61K 47/64; A61P 37/06; C12N 15/70

(52) U.S. Cl. CPC A61K 39/00114; A61K 39/385; A61K 47/646; A61P 37/06; C12N 15/70; A61K 2039/5258; A61K 2039/552; A61K 2039/575; A61K 2039/6075; A61K 2039/627; C12N 2770/14022; C12N 2770/14023; C12N 2770/14034

(57) ABSTRACT

The present invention relates to compositions comprising modified virus-like particles (VLPs) of Cucumber Mosaic Virus (CMV), and in particular to modified VLPs of CMV comprising chimeric CMV polypeptides which comprise a stretch of consecutive negative amino acids selected from aspartic acid and/or glutamic acid to which specific Interleukin-1β mutein antigens, IL-1β-D145X antigens, are linked, as well as pharmaceutical compositions thereof, which compositions preferably serve as vaccines for generating immune responses, in particular antibody responses, against IL-1β.

### CROSS-REFERENCE TO RELATED APPLICATIONS

This application claims priority to U.S. provisional application Ser. No. 63/562,153 filed Mar. 6, 2024, the contents of which is incorporated by reference.

### REFERENCE TO SEQUENCE LISTING SUBMITTED AS AN XML FILE (.xml)

A Sequence Listing is submitted herewith as an XML file named “P6731PC00_Sequence_Listing_ST26.xml” created on 27 Feb. 2025 and having a size of 62,556 bytes as permitted under 37 C.F.R. § 1.821 (c). The material in the aforementioned file is hereby incorporated by reference in its entirety.

### PARTIES TO A JOINT RESEARCH AGREEMENT

The presently disclosed invention was made by or on behalf of the below listed parties to a joint research agreement. The joint research agreement was in effect on or before the effective filing date of the disclosed invention, and the disclosed invention was made as a result of activities undertaken within the scope of the joint research agreement. The parties to the joint research agreement are Elanco US Inc. and SAIBA Animal Health AG.

### BACKGROUND

Virus-like particles (VLPs) have become an established and accepted vaccine technology, in particular as immunological carriers for inducing strong immune responses against conjugated antigens (Zeltins A, Mol Biotechnol (2013) 53:92-107; Jennings G T and Bachmann M F, Annu Rev Pharmacol Toxicol (2009) 49:303-26, Jennings G T and Bachmann M F, Biol Chem (2008) 389:521-536).

Recently, a vaccine platform based on Cucumber Mosaic Virus (CMV, family Bromoviridae, genus Cucumovirus) virus-like particles (CMV VLPs) has been described using chemical linker coupling technology to present different antigens, including self-antigens such as cytokines, on their surface, and elicit neutralizing antibody responses. These soluble CMV VLPs serve as an acceptable platform due to their intrinsic properties such as repetitive presentation of the target antigen to B cell receptors, nanoscale dimensions and geometry, as well as activation of innate immunity through activation of TLRs and provision of T cell help (WO2016/062720; U.S. Pat. No. 10,532,107; Zeltins A et al. Vaccines 2 (2017) 30; Bachmann M F et al. Frontiers in Microbiology Vol. 9, Article 2522 October 2018; von Loga I S et al. Ann. Rheum Dis 2019, 78:672-675; WO2021/260131).

However, despite the progress made in the course of the development of these VLP based vaccines, there remain unsolved challenges and requirements that have to be taken into account, in particular for eventual clinical trial testing, product registration, market launch and commercial supply needs. Hereby, controlling product characteristics such as stability, shelf-life, solubility, manufacturability including scalability, safety, potency, bioavailability and other pharmacological properties are particularly to be mentioned and are key elements of the chemistry, manufacturing and control (CMC) process necessary for the cost-effective provision of these products in sufficient amounts for such eventual clinical trial testing, product registration, market launch and commercial supply needs (Pham N G, Int J Pharm, 2020, 585:119523). In particular, the stability of VLP platforms and VLP based vaccines under various conditions required for an efficient CMC process is of relevance. A further undesired occurrence and problem negatively impacting product characteristics is the aggregation of biopharmaceuticals and vaccines, respectively (Roberts C J, Current Opinion in Biotechnology, 2014, 30:211-217). While an aggregated vaccine may still be capable of eliciting an immune response, provided its native structure is maintained, and even though it may thus still be suitable for some laboratory studies, it is not acceptable for GMP products produced for clinical studies and the market.

Therefore, despite the progress made in the course of the development of these VLP based vaccines, there is still a need for development of modified VLP systems adapted to address such challenges and to meet the requirements for eventual product registration and market launch.

Interleukin 1β is a pro-inflammatory cytokine. (WO 2008/037504; U.S. Pat. No. 8,449,874). The term “interleukin-1β (IL-1β)” may also be referred herein interchangeably to as “interleukin-1β”, “IL-1β”, “interleukin-1beta (IL-1beta)”, “interleukin-1beta”, “IL-1beta”, “interleukin-1b (IL-1b)”, “interleukin-1b”, or “IL-1b”.

### SUMMARY OF THE INVENTION

The inventors have surprisingly found that the inventive compositions comprising the modified VLPs of CMV to which specific Interleukin-1β mutein antigens are linked are not only highly immunogenic and lead to the induction of high titers of neutralizing antibodies against said Interleukin-1β mutein antigens in vitro, but, in addition, the inventive CMV VLP-IL-1β-D145X conjugates, in particular the CMV VLP-IL-1β-D145K conjugates retain their stability and structural integrity. This was in particular surprising since inclusion of additional negative charges within the VLP-forming proteins such as the inserted stretches of consecutive negative amino acids selected from glutamic acid and/or aspartic acid according to the present invention would have been expected to have deleterious effects on the formation of VLPs. In contrast, the present inventors have found not only that the specific insertion of these stretches of consecutive negative amino acids lead to improvements in stability of the resulting modified CMV VLPs as compared to prior art CMV VLPs under conditions of elevated temperatures and higher ionic strengths, but, moreover, that the inventive CMV VLP-IL-1β-D145X conjugates, in particular the CMV VLP-IL-1β-D145K conjugates, did not form aggregates and remained stable in solution upon linking the IL-1β-D145X antigens. In contrast, prior art CMV VLPs formed large aggregates and precipitated upon such linking. Such aggregation and formation of aggregated conjugated CMV VLPs is highly undesired for drug development and product registration, and the substantial reduction or avoidance of such undesired aggregation by the inventive compositions is highly beneficial. Furthermore, the improved stability in higher salt solution arising from the surface charge modifications to the inventive CMV VLPs is additionally beneficial or even essential for their processability and purification by ion-exchange chromatography, in particular anion exchange chromatography, which advantageously further allows readiness for scalable manufacturing of the inventive compositions.

Moreover, the inventors have surprisingly found that the preferred muteins of IL-1 beta show a reduced natural biological activity, but when linked to the modified CMV VLPs forming the inventive compositions are highly capable of inducing anti-IL-1 beta antibodies with neutralizing activity in vitro and in vivo. Furthermore, the present inventors have discovered that the administration of the inventive composition can be produces only very mild adverse effects of very short duration.

Thus, in a first aspect, the present invention provides a composition, preferably a veterinary composition, comprising
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least about 75% with SEQ ID NO:45; and
   (ii) optionally a polypeptide comprising, preferably consisting of, a stretch of consecutive negative amino acids, wherein said negative amino acids are independently selected from aspartic acid and/or glutamic acid, wherein said polypeptide is inserted between any two adjacent amino acid residues of said CMV polypeptide corresponding to any two adjacent amino acid residues between position 75 and position 85 of SEQ ID NO:45.
(b) at least one antigen, wherein said antigen comprises at least one second attachment site, and wherein said antigen is an interleukin 1β D145X mutein (IL-1β-D145X mutein) antigen, preferably an interleukin 1β D145K mutein (IL-1β-D145K mutein) antigen; and
   wherein (a) and (b) are linked through said at least one first and said at least one second attachment site, preferably via at least one covalent non-peptide bond.

Further aspects and embodiments of the present invention will become apparent as this description continues.

### BRIEF DESCRIPTION OF FIGURES

FIG. 1: Description of pET-CMVB2-Ntt-E8* plasmid map with single-cut restriction enzyme sites.

FIG. 2A: SDS-PAGE gel analysis of the purification of the VLP derived from the expression of CMV-Ntt830-E8*. M—protein size marker PageRuler (Thermo Fisher Scientific, #26620); S—soluble proteins in cell extract in E. coli C2566/pET-CMVB2-Ntt-E8*; P—insoluble proteins in cell extract; 1—insoluble proteins after sucrose gradient (bottom of the tube); 2-6—sucrose gradient fractions (from 60% at the bottom of tube to 0% at the top). The asterisk (*) within the figure denotes the relative position of the corresponding CMV-Ntt830-E8* chimeric CMV polypeptide in SDS/PAGE gel.

FIG. 2B: Electron microscopy images of purified CMV-Ntt830-E8* VLPs. The horizontal bar corresponds to 500 nm.

FIG. 3: Description of pET-CMVB2-Ntt-E4 plasmid map with single-cut restriction enzyme sites.

FIG. 4: Description of pET-CMVB2-Ntt-E8 plasmid map with single-cut restriction enzyme sites.

FIG. 5: Description of pET-CMVB2-Ntt-E12 plasmid map with single-cut restriction enzyme sites.

FIG. 6: SDS-PAGE (left) and agarose gel (right) analysis of the purification of the VLP derived from the expression of CMV-Ntt830-E4. M1—protein size marker PageRuler (Thermo Fisher Scientific, #26620); M2—DNA size marker (Thermo Fisher Scientific, #SM0311); T—total proteins in E. coli C2566 cells after 18 h cultivation at 20° C.; S—soluble proteins in cell extract after cell disruption before sucrose gradient (20-60%); P—insoluble proteins; 1-6—sucrose gradient fractions (from 60% at the bottom of tube to 0% at the top. The asterisk (*) within the figure denotes the relative position of the corresponding CMV-Ntt830-E4 chimeric CMV polypeptide in SDS/PAGE gel and typical VLP signal in agarose gel.

FIG. 7: SDS-PAGE (left) and agarose gel (right) analysis of the purification of the VLP derived from the expression of CMV-Ntt830-E8. M1—protein size marker PageRuler (Thermo Fisher Scientific, #26620); M2—DNA size marker (Thermo Fisher Scientific, #SM0311); T—total proteins in E. coli C2566 cells after 18 h cultivation at 20° C.; S—soluble proteins in cell extract after cell disruption before sucrose gradient (20-60%); P—insoluble proteins; 1-6—sucrose gradient fractions (from 60% at the bottom of tube to 0% at the top. The asterisk (*) within the figure denotes the relative position of the corresponding CMV-Ntt830-E8 chimeric CMV polypeptide in SDS/PAGE gel and typical VLP signal in agarose gel.

FIG. 8: SDS-PAGE (left) and agarose gel (right) analysis of the purification of the VLPs derived from the expression of CMV-Ntt830-E12. M1—protein size marker PageRuler (Thermo Fisher Scientific, #26620); M2—DNA size marker (Thermo Fisher Scientific, #SM0311); T—total proteins in E. coli C2566 cells after 18 h cultivation at 20° C.; S—soluble proteins in cell extract after cell disruption before sucrose gradient (20-60%); P—insoluble proteins; 1-6—sucrose gradient fractions (from 60% at the bottom of tube to 0% at the top. The asterisk (*) within the figure denotes the relative position of the corresponding CMV-Ntt830-E12 chimeric CMV polypeptide in SDS/PAGE gel. A clear and distinct band corresponding to intact VLPs was not observed in the agarose gel.

FIG. 9: Electron microscopy images of purified CMV-Ntt830-E4 VLPs. Horizontal bar corresponds to 200 nm.

FIG. 10: Electron microscopy images of purified CMV-Ntt830-E8 VLPs. Horizontal bar corresponds to 200 nm.

FIG. 11: Comparison of thermal stability of CMV-Ntt830 VLPs and CMV-Ntt830-E4 VLPs. The structural changes in CMV-Ntt830 VLPs and CMV-Ntt830-E4 VLPs were monitored in the presence of Sypro-Orange dye using a DNA melting point determination program and a real-time PCR system. Curve 1 is for CMV-Ntt830-E4 VLPs), curve 2 is for CMV-Ntt830 VLPs and Curve 3 is for buffer control (5 mM Na phosphate 2 mM EDTA, pH 7.5). The respective 57° C. and 51° C. melting points are indicated by arrows.

FIG. 12: Stability of CMV-Ntt830 VLPs and CMV-Ntt830-E4 VLPs in solution in the presence of different NaCl concentrations. Samples of CMV-Ntt830 VLPs and CMV-Ntt830-E4 VLPs at 0.5 mg/ml were incubated at room temperature in 5 mM Na phosphate, 2 mM EDTA, pH 7.5 with different concentrations of NaCl (the molar concentration of NaCl in each sample is indicated at the bottom of the gels) for up to 2 hours. Samples were analysed by native agarose gel electrophoresis and ethidium bromide staining. Panels A and B show NAGE analysis of CMV-Ntt830 VLP and CMV-Ntt830-E4 VLPsamples respectively. M shows the lanes loaded with GeneRuler 1 kb DNA Ladder (SM0311, TFS). Black arrows indicate the position of loading wells within the gels and location of VLPs within the wells and gels. The presence of CMV-Ntt830 VLPs in the loading wells after electrophoresis (panel A) is due to the formation of VLP aggregates which are too large to enter the gel. Integral unaggregated VLPs migrated into the gel.

FIG. 13: Analysis of CMV-Ntt830 VLPs subject to Anion Exchange Chromatography. 5 ml of 1 mg/ml CVMtt-VLPs in 5 mM Sodium Borate buffer pH 9.0 was loaded onto 1.0 ml Macro-Prep DEAE Bio-Rad anion exchange cartridge equilibrated with 5 mM Sodium Borate buffer and eluted step-wise with increasing concentrations of NaCl (0.1, 0.2, 0.3, 0.4. 0.5, 0.8, 1.0 and 2.0 M). Fractions were collected and analysed by nanodrop 260 nm for protein concertation and native agarose gel electrophoresis. Panel A shows the NaCl concentration and 260 nm absorbance values plotted against the respective fractions (1-25). Panel B is a NAGE analysis (ethidium bromide stained) of the principle fractions containing the highest protein concentrations. M shows the lanes loaded with GeneRuler 1 kb DNA Ladder (SM0311, TFS). Black arrows indicate the position of loading wells within the gels and location of VLPs within the wells and gels. The presence of CMV-Ntt830 VLPs in the loading wells after electrophoresis is due to the formation of VLP aggregates which are too large to enter the gel. Integral unaggregated VLPs migrated into the gel.

FIG. 14: Analysis of CMV-Ntt830-E4 VLPs subject to Anion Exchange Chromatography. A biomass of E. coli cells expressing CMV-Ntt830-E4 VLPs was resuspended in 50 mM citrate, 5 mM Borate buffer pH 9.0 and cells were lysed using a microfluidizer LM-20. The soluble fraction was clarified by centrifugation and loaded onto a 60 ml Fracto-DEAE (XK 26/20). An elution buffer comprising 50 mM Citrate 5 mM Borate and 1M NaCl was applied in a continuous gradient manner to elute the bound VLPs. Panel A shows the protein elution and NaCl concentration gradient measured by A260 nm (mAU) and conductivity (mS/cm) respectively. The X-axis shows the elution volume and fraction numbers (4-11). The fractions collected from the Fracto-DEAE column were analysed by NAGE (panel B) and SDS-PAGE (panel C). In panel B, M indicates the lane loaded with a GeneRuler 1 kb DNA Ladder (SM0311, TFS), L is a sample of E. coli lysate before loading onto the Fracto DEAE, FT is the flow through collected from 0 to 150 ml and 4-10 represent the fraction numbers collected during elution. The black arrows from top to bottom indicate the position of the loading wells, position of integral CMV-Ntt830-E4 VLPs within the gel and contaminating nucleic acids from the clarified bacterial lysate respectively. In panel C, FT is the flow through collected from 0 to 150 ml and 4-10 represent the fraction numbers. The black arrow shows the position of the Coomassie blue stained CMV-Ntt830-E4 coat protein.

FIG. 15: Description of pET42NBS-cIL1b-C6Hcg plasmid map with single-cut restriction enzyme sites.

FIG. 16A: SDS-PAGE analysis of E. coli C2566/pET42NBS-cIL1b-D145K-C6Hcg expression clones. E. coli C2566 cells transformed with the pET42NBS-cIL1b-D145K-C6Hcg expression plasmid were grown on agar plates and three single colonies picked (clone 1, clone 2, clone 3). Selected clones were grown in growth medium at 30° C. to an OD600 of 0.8-1.0. A sample was collected (0) and expression of canine IL-1β-D145K was induced by addition of IPTG and MgCl₂ for 18 hours at +20° C. The cells were harvested and lysed. The expression of canine IL-1β-D145K at equal levels by all three clones was confirmed by loading the total lysate (T) prior to clarification onto an SDS-PAGE. The arrow indicates expected protein size of canine IL-1β-D145K.

FIG. 16B: SDS-PAGE analysis of the HisTag column purification by Protino, Ni-IDA columns. After expression the clarified lysate was subjected to immobilized metal ion affinity chromatography (IMAC) column (Protino, Ni-IDA) and a Superdex 75 gel filtration purification. M—protein size marker PageRuler (Thermo Fisher Scientific, #26620); S—soluble fraction of E. coli C2566/pET42NBS-cIL1b-D145K-C6Hcg cell extract after 18 h induction of canine IL-1β-D145K at +20° C., lysis and clarification prior to loading onto IMAC column purification; P—insoluble fraction of E. coli C2566/pET42NBS-CIL1b-D145K-C6Hcg cell extract after 18 h induction of canine IL-1β-D145K at +20° C., lysis and clarification; FT—flow through sample of the IMAC column purification; W1—fraction of IMAC column purification collected during the first column wash; W2—fraction of IMAC column purification collected during the second column wash; E1—fraction of IMAC column purification collected after the first addition of elution buffer; E2—fraction of IMAC column purification collected after the second addition of elution buffer; E3—fraction of IMAC column purification collected after the third addition of elution buffer. The arrow indicates expected protein size of canine IL-1β-D145K.

FIG. 17: SDS-PAGE analysis of fractions collected during the size exclusion chromatography using a Superdex 75 column. After purification by IMAC columns the elution fraction 1 containing the canine IL-1β-D145K was subjected a Superdex 75 gel filtration purification. Fractions collected during the Superdex column purification run (1-4) containing canine IL-1β-D145K were subjected to an SDS-PAGE analysis. M—protein size marker PageRuler (Thermo Fisher Scientific, #26620); The arrow indicates expected protein size of canine IL-1β-D145K.

FIG. 18A: canine IL-1β-D145K testing by a canine IL-1β-specific ELISA. The authenticity of the canine IL-1β-D145K was confirmed by a canine IL-1β-specific ELISA. Purified canine IL-1β-D145K mutein (triangle), purified canine IL-1β wild type (squares) and recombinant canine IL-1β purchased from R&D system (circles) were tested.

FIG. 18B: canine IL-1β-D145K testing by a canine IL-1β/IL-1RI-binding ELISA. The ability of the canine IL-1β-D145K mutein to bind the IL-1RI was confirmed by a IL-1β/IL-1RI-binding ELISA. Purified canine IL-1β-D145K mutein (triangle), purified canine IL-1β wild type (squares) and recombinant canine IL-1β purchased from R&D system (circles) were tested.

FIG. 18C: Bioactivity testing of canine IL-1β-D145K mutein using the HEK-Blue IL-1beta reporter cells. HEK-Blue™ IL-1 cells were incubated for 20 hours in presence of purified canine IL-1β-D145K mutein (triangle), purified canine IL-1β wild type (squares) and recombinant canine IL-1β purchase from R&D system (circles). The reporter protein, secretory alkaline phosphatase (SAP), in the culture supernatant was quantitated by QUANTI-Blue Solution. Absorbance at 620 nm was measured 5 hours after addition of substrate solution.

FIG. 19A: SDS-PAGE analyses of expression and purification of cIL1b-D145K-CMV-Ntt830-E4 VLPs. The indicated lanes were loaded with the following samples: M—Page Ruler™ Plus (Pre-stained Protein Ladder, #26620 Thermo Fisher Scientific); 1—CMV-Ntt830-E4 VLPs; 2—CMV-Ntt830-E4 VLPs after 5×SMPH derivatization and unreacted SMPH removal; 3—CMV-Ntt830-E4 VLPs after coupling with 0.25× canine IL-1β-D145K treated with 10×TCEP; 4—soluble fraction of CMV-Ntt830-E4 VLPs after coupling with 0.25× IL-1β-D145K (10×TCEP) and centrifugation at 14,000 rpm for 5 minutes; 5—insoluble fraction of CMV-Ntt830-E4 VLPs after coupling with 0.25× IL-1β-D145K (10×TCEP) and centrifugation at 14,000 rpm for 5 minutes; 6—cIL1b-D145K-CMV-Ntt830-E4 after ultrafiltration with Amicon-Ultra-15 filtration units, 100K. The asterisk denotes the position of cIL1b-D145K-CMV-Ntt830-E4 coupling product in the SDS-PAGE gel.

FIG. 19B: Native agarose gel electrophoresis stained with ethidium bromide of samples collected during expression and purification of cIL1b-D145K-CMV-Ntt830-E4 VLPs. The indicated lanes were loaded with the following samples: M-Gene Ruler™ 1 kb DNA Ladder (#SM0311 Thermo Fisher Scientific); 2—CMV-Ntt830-E4 VLPs after 5×SMPH derivatization and unreacted SMPH removal; 3—CMV-Ntt830-E4 VLPs after coupling with 0.25× canine IL-1β-D145K treated with 10×TCEP; 4—soluble fraction of CMV-Ntt830-E4 VLPs after coupling with 0.25× IL-1β-D145K (10×TCEP) and centrifugation at 14,000 rpm for 5 minutes; 6—cIL1b-D145K-CMV-Ntt830-E4 after ultrafiltration with Amicon-Ultra-15 filtration units, 100K.

FIG. 19C: Native agarose gel electrophoresis stained with Coomassie Blue of samples collected during expression and purification of cIL1b-D145K-CMV-Ntt830-E4 VLPs. The indicated lanes were loaded with the following samples: M-Gene Ruler™ 1 kb DNA Ladder (#SM0311 Thermo Fisher Scientific); 2—CMV-Ntt830-E4 VLPs after 5×SMPH derivatization and unreacted SMPH removal; 3—CMV-Ntt830-E4 VLPs after coupling with 0.25× canine IL-1β-D145K treated with 10×TCEP; 4—soluble fraction of CMV-Ntt830-E4 VLPs after coupling with 0.25× IL-1β-D145K (10×TCEP) and centrifugation at 14,000 rpm for 5 minutes; 6—cIL1b-D145K-CMV-Ntt830-E4 after ultrafiltration with Amicon-Ultra-15 filtration units, 100K.

FIG. 20A: Dynamic light scattering analysis of cIL1b-D145K-CMV-Ntt830-E4 VLPs.

FIG. 20B: Electron microscopic analysis of cIL1b-D145K-CMV-Ntt830-E4 VLPs.

FIG. 21A: cIL-1b-D145K-CMV-Ntt830-E4 VLPs testing using a canine IL-1β-specific ELISA. The authenticity of the canine IL-1β-D145K coupled to the CMV-Ntt830-E4 VLPs was confirmed by a canine IL-1β-specific ELISA. cIL1b-D145K-CMV-Ntt830-E4 VLPs (squares) and control CMV-Ntt830-E4 VLPs (open diamonds) were tested.

FIG. 21B: cIL-1b-D145K-CMV-Ntt830-E4 VLPs testing using canine IL-1β/IL-1RI-binding ELISA. The authenticity of the canine IL-1β-D145K coupled to the CMV-Ntt830-E4 VLPs was confirmed by canine IL-1β/IL-1RI-binding ELISA. cIL1b-D145K-CMV-Ntt830-E4 VLPs (squares) and control CMV-Ntt830-E4 VLPs (open diamonds) were tested.

FIG. 21C: cIL-1b-D145K-CMV-Ntt830-E4 VLPs testing using a HEK-Blue IL-1beta reporter cell based bioactivity assay. The loss of bioactivity of the cIL1b-D145K-CMV-Ntt830-E4 VLPs was confirmed using HEK-Blue IL-1beta reporter cells. cIL1b-D145K-CMV-Ntt830-E4 VLPs (squares) and control CMV-Ntt830-E4 VLPs (open diamonds) were tested. The wild type canine IL-1β recombinant protein conjugated to the CMV-Ntt830-E4 VLPs was included as a positive control (circles).

FIG. 22: SDS-PAGE analysis of the HisTag column purification of the feline IL-1β-D145K antigen by Protino, Ni-IDA columns. After expression the clarified lysate was subjected to immobilized metal ion affinity chromatography (IMAC) column (Protino, Ni-IDA) and a Superdex 75 gel filtration purification. M—protein size marker PageRuler (Thermo Fisher Scientific, #26620); T0—E. coli C2566/pET42NBS-fIL1b-145K-C6Hcg sample prior to addition of IPTG to the culture and induction of feline IL-1β-D145K expression. S—soluble fraction of E. coli C2566/pET42NBS-fIL1b-145K-C6Hcg cell extract after 18 h induction of feline IL-1β-D145K at +20° C., lysis and clarification prior to loading onto IMAC column purification; P—insoluble fraction of E. coli C2566/pET42NBS-fIL1b-145K-C6Hcg cell extract after 18 h induction of feline IL-1β-D145K at +20° C., lysis and clarification; FT—flow through sample of the IMAC column purification; W1—fraction of IMAC column purification collected during the first column wash; W2—fraction of IMAC column purification collected during the second column wash; E1—fraction of IMAC column purification collected after the first addition of elution buffer; E2—fraction of IMAC column purification collected after the second addition of elution buffer; E3—fraction of IMAC column purification collected after the third addition of elution buffer. The arrow indicates expected protein size of feline IL-1β-D145K.

FIG. 23: SDS-PAGE analysis of fractions containing feline IL-1β-D145K collected during the size exclusion chromatography using a Superdex 75 column. After purification by IMAC columns the elution fraction 1 containing the feline IL-1β-D145K was subjected a Superdex 75 gel filtration purification. Fractions collected during the Superdex column purification run (2 and 3) containing feline IL-1β-D145K were subjected to an SDS-PAGE analysis. M—protein size marker PageRuler (Thermo Fisher Scientific, #26620); The arrow indicates expected protein size of feline IL-1β-D145K.

FIG. 24A: feline IL-1β-D145K testing using a feline IL-1β-specific ELISA. The authenticity of the feline IL-1β-D145K was confirmed by a feline IL-1β-specific ELISA. Purified feline IL-1β-D145K mutein (triangle), purified feline IL-1β wild type (squares) and recombinant feline IL-1β purchased from R&D system (circles) were tested.

FIG. 24B: feline IL-1β-D145K testing using a feline IL-1β/IL-1RI-binding ELISA. The authenticity of the feline IL-1β-D145K was confirmed by a feline IL-1β/IL-1RI-binding ELISA. Purified feline IL-1β-D145K mutein (triangle), purified feline IL-1β wild type (squares) and recombinant feline IL-1β purchased from R&D system (circles) were tested.

FIG. 24C: feline IL-1β-D145K testing using a HEK-Blue IL-1beta reporter cell-based bioactivity assay. The loss of bioactivity of the feline IL-1β-D145K was confirmed using a HEK-Blue IL-1beta reporter cell line. Purified feline IL-1β-D145K mutein (triangle), purified feline IL-1β wild type (squares) and recombinant feline IL-1β purchased from R&D system (circles) were tested.

FIG. 25A: SDS-PAGE analyses of samples collected during expression and purification of the fIL-1b-D145K-CMV-Ntt830-E4 VLPs. The indicated lanes were loaded with the following samples: M1—Page Ruler™ Plus (Pre-stained Protein Ladder, #26620 Thermo Fisher Scientific); 1—CMV-Ntt830-E4 VLP; 2—CMV-Ntt830-E4 after 5×SMPH derivatization and unreacted SMPH removal; 3—CMV-Ntt830-E4 after coupling with 0.3× feline IL-1β-D145K treated with 10×TCEP; 4—soluble fraction of CMV-Ntt830-E4 after coupling with fIL-1β-D145K (10×TCEP) and centrifugation at 14,000 rpm for 5 minutes; 5—fIL-1b-D145K-CMV-Ntt830-E4 after removal of fIL-1β-D145K not associated with the CMV-Ntt830-E4 VLPs; 6—fIL-1b-D145K-CMV-Ntt830-E4 after sterile filtration using an 0.2 μm filter; 7—purified feline IL-1β-D145K. The asterisk denotes the position of fIL-1b-D145K-CMV-Ntt830-E4 coupling product in the SDS-PAGE gel.

FIG. 25B: Native agarose gel stained with ethidium bromide of samples collected during expression and purification of the fIL-1b-D145K-CMV-Ntt830-E4 VLPs. The indicated lanes were loaded with the following samples: M2—Gene Ruler™ 1 kb DNA Ladder (#SM0311 Thermo Fisher Scientific); 1—CMV-Ntt830-E4 VLP; 2—CMV-Ntt830-E4 after 5×SMPH derivatization and unreacted SMPH removal; 3—CMV-Ntt830-E4 after coupling with 0.3× feline IL-1β-D145K treated with 10×TCEP; 4—soluble fraction of CMV-Ntt830-E4 after coupling with fIL-1β-D145K (10×TCEP) and centrifugation at 14,000 rpm for 5 minutes; 5—fIL-1b-D145K-CMV-Ntt830-E4 after removal of fIL-1β-D145K not associated with the CMV-Ntt830-E4 VLPs; 6—fIL-1b-D145K-CMV-Ntt830-E4 after sterile filtration using an 0.2 μm filter.

FIG. 25C: Native agarose gel stained with Coomassie Blue of samples collected during expression and purification of the fIL-1b-D145K-CMV-Ntt830-E4 VLPs. The indicated lanes were loaded with the following samples: M2—Gene Ruler™ 1 kb DNA Ladder (#SM0311 Thermo Fisher Scientific); 1—CMV-Ntt830-E4 VLP; 2—CMV-Ntt830-E4 after 5×SMPH derivatization and unreacted SMPH removal; 3—CMV-Ntt830-E4 after coupling with 0.3× feline IL-1β-D145K treated with 10×TCEP; 4—soluble fraction of CMV-Ntt830-E4 after coupling with fIL-1β-D145K (10×TCEP) and centrifugation at 14,000 rpm for 5 minutes; 5—fIL-1b-D145K-CMV-Ntt830-E4 after removal of fIL-1β-D145K not associated with the CMV-Ntt830-E4 VLPs; 6—fIL-1b-D145K-CMV-Ntt830-E4 after sterile filtration using an 0.2 μm filter.

FIG. 26A: Dynamic light scattering analysis of fIL1b-D145K-CMV-Ntt830-E4 VLPs.

FIG. 26B: Electron microscopic analysis of fIL1b-D145K-CMV-Ntt830-E4 VLPs.

FIG. 27A: fIL-1b-D145K-CMV-Ntt830-E4 VLPs testing using a feline IL-1β-specific ELISA. The authenticity of the feline IL-1β-D145K coupled to the CMV-Ntt830-E4 VLPs was confirmed by a feline IL-1β-specific ELISA. fIL1b-D145K-CMV-Ntt830-E4 VLPs (squares) and control CMV-Ntt830-E4 VLPs (diamonds) were tested. The wild type feline IL-1β recombinant protein conjugated to the CMV-Ntt830-E4 VLPs was included as a positive control (circles).

FIG. 27B: fIL-1b-D145K-CMV-Ntt830-E4 VLPs testing using canine IL-1β/IL-1RI-binding ELISA. The authenticity of the feline IL-1β-D145K coupled to the CMV-Ntt830-E4 VLPs was confirmed by feline IL-1β/IL-1RI-binding ELISA. fIL1b-D145K-CMV-Ntt830-E4 VLPs (squares) and control CMV-Ntt830-E4 VLPs (diamonds) were tested. The wild type feline IL-1β recombinant protein conjugated to the CMV-Ntt830-E4 VLPs was included as a positive control (circles).

FIG. 27C: fIL-1b-D145K-CMV-Ntt830-E4 VLPs testing using a HEK-Blue IL-1beta reporter cell based bioactivity assay. The loss of bioactivity of the fIL1b-D145K-CMV-Ntt830-E4 VLPs was confirmed using HEK-Blue IL-1beta reporter cells. fIL1b-D145K-CMV-Ntt830-E4 VLPs (squares) and control CMV-Ntt830-E4 VLPs (diamonds) were tested. The wild type feline IL-1β recombinant protein conjugated to the CMV-Ntt830-E4 VLPs was included as a positive control (circles).

FIG. 28A: Kinetics of the anti-fIL-1β in mice administered with the fIL1b-D145K-CMV-Ntt830-E4 vaccine on two occasions. 5 Balb/c mice were dosed on two occasions three weeks apart, on day 0 and on day 21, with 30 μg per dose of fIL1b-D145K-CMV-Ntt830-E4. Sera were collected on day 0, day 14, day 21, day 35 and day 42. fIL-1β-binding IgG antibodies were measured in the sera. Geometric mean titers of the study groups were graphed as EC50 values. The error bars represent the 95% CI of the geometric mean titer (GMT). Assay results below the level of detection were set to 50, corresponding to 0.5× the lowest dilution factor used in the assays.

FIG. 28B: Kinetics of the anti-CMV IgG responses in mice administered with the fIL1b-D145K-CMV-Ntt830-E4 vaccine on two occasions. 5 Balb/c mice were dosed on two occasions three weeks apart, on day 0 and on day 21, with 30 μg per dose of fIL1b-D145K-CMV-Ntt830-E4. Sera collected on day 0, day 14, day 21, day 35 and day 42 were used to determine the CMV carrier-specific IgG antibody titers. Geometric mean titers (GMTs) of the study groups were graphed as EC50 values. The error bars represent the 95% CI of the GMT. Assay results below the level of detection were set to 50, corresponding to 0.5× the lowest dilution factor used in the assays.

FIG. 28C: Kinetics of the fIL-1β neutralization titers in mice administered with the fIL1b-D145K-CMV-Ntt830-E4 vaccine on two occasions. 5 Balb/c mice were dosed on two occasions three weeks apart, on day 0 and on day 21, with 30 μg per dose of fIL1b-D145K-CMV-Ntt830-E4. Sera collected on day 0, day 21 and day 42 after first dosing were used to determine fIL-1β neutralizing antibodies induced by the vaccine. Geometric mean titers (GMT) of the study groups were graphed as EC50 values. The error bars represent the 95% CI of the GMT. Assay results below the level of detection were set to 25, corresponding to 0.5× the lowest dilution factor used in the assays.

FIG. 29 shows systemic observations pre- and post vaccination of cats with an exemplary composition described herein. The alphabetical scale utilized is as follows:
A=Normal
B=Generalized scratching/biting/rubbing
C=Vocalization
D=Labored breathing
E=Lethargy/depression
F=Vomiting
G=Inappetence
H=Diarrhea
I=Facial Swelling
J=Hives
K=Other

FIG. 30 shows local injection site reaction observations pre- and post vaccination of cats with an exemplary composition described herein. The numerical scale utilized is as follows:
0=No reactions
1=Pain upon palpation
2=Injection site markedly warmer than rest of the body
3=Swelling
4=Mild lump <1 cm
5=Moderate lump 1-3 cm
6=Severe lump >3 cm
7=Abscess (with drainage)
8=Scratching/biting/rubbing of injection site
9=Other
*=No observations conducted per protocol

FIG. 31 shows, in degrees Celsius, rectal temperatures pre- and post vaccination of cats with an exemplary composition described herein. Pyrexia is indicated by a temperature >39. %° C. and at least 0.6° C. above baseline.

FIG. 32 shows, in degrees Fahrenheit, rectal temperatures pre- and post vaccination of cats with an exemplary composition described herein. Pyrexia is indicated by a temperature >103.5° F. and at least 1.0° F. above baseline.

FIG. 33 shows the results of feline IL-1β total antibody ELISA assays performed on (pre- and post-vaccination) sera from cats vaccinated with an exemplary composition described herein. All cats were screened on SD-8 for total feline IL-1β antibodies using an ELISA method prior to being enrolled in the study and were found to be negative for IL-1β antibodies. Cats were vaccinated with a primary vaccination on SD0. All cats had detectable feline IL-1β antibodies by SD20. Antibody titers increased following the two booster vaccinations on SD21 and SD42 with peak antibody titers observed on SD 40, 80, and 154.

FIG. 34 shows the results of feline IL-1β serum neutralization assays on (pre- and post-vaccination) sera from cats vaccinated with an exemplary composition described herein. Prior to being enrolled in the study, all cats were screened on study day (SD)-8 for IL-1β serum neutralization (SN) titers. None of the cats had detectable IL-1β serum neutralization (SN) titers prior to study initiation. After receiving the initial primary vaccination on SD0, all cats remained negative for IL-1β SN titers through SD20. On SD21, all cats received the first booster vaccination, and an IL-1 β SN response was observed in all cats on SD40. The 2nd and final booster vaccination was administered on SD42. On SD80 the IL-1β SN response increased further and the titers remained high through the remainder of the study.

### DETAILED DESCRIPTION OF THE INVENTION

Virus-like particle (VLP): The term “virus-like particle (VLP)” as used herein, refers to a non-replicative and/or non-infectious, preferably a non-replicative and non-infectious virus particle, or refers to a non-replicative and/or non-infectious, preferably a non-replicative and non-infectious structure resembling a virus particle, preferably a capsid of a virus. The term “non-replicative”, as used herein, refers to being incapable of replicating the genome comprised by the VLP. The term “non-infectious”, as used herein, refers to being incapable of entering the host cell. A virus-like particle in accordance with the invention preferably is non-replicative and non-infectious since it lacks all or part of the viral genome or genome function. A virus-like particle in accordance with the invention may contain nucleic acid distinct from its genome. Recombinantly produced virus-like particles typically contain host cell derived RNA. A typical and preferred embodiment of a virus-like particle in accordance with the present invention is a viral capsid comprising or composed of polypeptides of the invention. A virus-like particle is typically a macromolecular assembly composed of viral coat protein which typically comprises 60, 120, 180, 240, 300, 360, or more than 360 protein subunits per virus-like particle. Typically, and preferably, the interactions of these subunits lead to the formation of viral capsid or viral capsid-like structure with an inherent repetitive organization. One feature of a typical virus-like particle is its highly ordered and repetitive arrangement of its subunits.

Modified virus-like particle (VLP) of CMV: The term “modified virus-like particle of CMV” refers to a virus-like particle comprising at least one chimeric CMV polypeptide as defined and as described herein. Typically and preferably, modified VLPs of CMV resemble the structure of the capsid of CMV. Modified VLPs of CMV are non-replicative and/or non-infectious, and lack at least the gene or genes encoding for the replication machinery of the CMV, and typically also lack the gene or genes encoding the protein or proteins responsible for viral attachment to or entry into the host. This definition includes also modified virus-like particles in which the aforementioned gene or genes are still present but inactive. Preferably, non-replicative and/or non-infectious modified virus-like particles are obtained by recombinant gene technology and typically and preferably do not comprise the viral genome. Preferably, a modified VLP of CMV is a macromolecular assembly comprising or composed of CMV polypeptides modified in accordance with the present invention, and typically and preferably comprising 180 of such protein subunits and chimeric polypeptides, respectively per VLP. Thus, in a preferred embodiment, said modified virus-like particle (VLP) of cucumber mosaic virus (CMV) comprises 180 chimeric CMV polypeptides. However, in some embodiments, a modified VLP of CMV may comprise a different number of chimeric CMV polypeptides, such as 60, 120, 240, 300, 360, or more than 360 chimeric CMV polypeptides.

Polypeptide: The term “polypeptide” as used herein refers to a polymer composed of amino acid monomers which are linearly linked by amide bonds (also known as peptide bonds). It indicates a molecular chain of amino acids and does not refer to a specific length of the product. Thus, peptides, dipeptides, tripeptides, oligopeptides and proteins are included within the definition of polypeptide. In embodiments, the term “polypeptide” as used herein also refers to a polypeptide as defined hereinabove and encompassing modifications such as post-translational modifications, including but not limited to glycosylations. In embodiments, the term “polypeptide” as used herein refers to a polypeptide as defined hereinabove and not encompassing modifications such as post-translational modifications such as glycosylations. In particular, for said biologically active peptides, said modifications such as said glycosylations can occur even in vivo thereafter, for example, by bacteria.

Cucumber Mosaic Virus (CMV) polypeptide, CMV polypeptide: The term “cucumber mosaic virus (CMV) polypeptide” as used herein refers to a polypeptide comprising or preferably consisting of: (i) an amino acid sequence of a coat protein of cucumber mosaic virus (CMV), or (ii) a mutated amino acid sequence, wherein said mutated amino acid sequence and said coat protein of CMV show a sequence identity of at least 90%, at least 91%, at least 92%, at least 93% or at least 94%, at least 95%, at least 98%, or at least 99%. Typically and preferably, the CMV polypeptide is capable of forming a virus-like particle of CMV upon expression by self-assembly.

Coat protein (CP) of cucumber mosaic virus (CMV): The term “coat protein (CP) of cucumber mosaic virus (CMV)”, as used herein, refers to a coat protein of the cucumber mosaic virus which occurs in nature. Due to extremely wide host range of the cucumber mosaic virus, many different strains and isolates of CMV are known. The sequences of the coat proteins of said strains and isolates have been determined and are known to the skilled person in the art. The sequences of said coat proteins (CPs) of CMV are described in and retrievable from the known databases such as Genbank, www.dpvweb.net, or www.ncbi.nlm.nih.gov/protein/. Specific examples CPs of CMV are described in WO2016/062720 at page 12, line 8 to page 13, line 25 (and at corresponding locations in U.S. Pat. No. 10,532,107), the disclosure of each of which is explicitly incorporated herein by way of reference. A very preferred example and embodiment of a CMV coat protein is provided in SEQ ID NO:45. Thus, in embodiments, the term “coat protein of cucumber mosaic virus (CMV)”, as used herein, refers to an amino acid sequence of a coat protein of CMV, wherein said amino acid sequence comprises, or preferably consists of, SEQ ID NO: 45 or an amino acid sequence having a sequence identity of at least 75%, at least 80%, at least 85%, at least 90%, at least 91%, at least 92%, at least 93%, at least 94%, at least 95%, at least at least 96%, or at least 97%, at least about 98%, or at least 99% of SEQ ID NO: 45.

It is noteworthy that these strains and isolates of CMV have highly similar coat protein sequences at different protein domains, such as the N-terminus of the coat protein. For example, 98.1% of all completely sequenced CMV isolates share more than 85% sequence identity within the first 28 amino acids of their coat protein sequence, and still 79.5% of all completely sequenced CMV isolates share more than 90% sequence identity within the first 28 amino acids of their coat protein sequence.

M-CMV polypeptide: The term “M-CMV polypeptide” as used herein refers to a CMV polypeptide comprising, or preferably consisting of, (i) a CMV polypeptide, and (ii) a T helper cell epitope. Typically, M-CMV polypeptide is capable of forming a virus-like particle of CMV upon expression by self-assembly. Typically and preferably, said T helper cell epitope replaces a N-terminal region of said CMV polypeptide, wherein said replaced N-terminal region of said CMV polypeptide consists of 5 to 15 consecutive amino acids. Preferably, said T helper cell epitope replaces a N-terminal region of said CMV polypeptide, and wherein said replaced N-terminal region of said CMV polypeptide consists of 5 to 15 consecutive amino acids, 9 to 14 consecutive amino acids, 9 to 13 consecutive amino acids, 10 to 13 consecutive amino acids, 11 to 13 consecutive amino acids, 11 consecutive amino acids, 12 consecutive amino acids, or 13 consecutive amino acids. Preferably, the M-CMV polypeptide is a recombinant M-CMV polypeptide and is capable of forming a virus-like particle of CMV upon expression by self-assembly in E. coli.

Chimeric CMV polypeptide: The term “chimeric CMV polypeptide” as used herein refers to a polypeptide as defined herein and in accordance with the present invention, and comprising, preferably consisting of a CMV polypeptide, wherein said CMV polypeptide is modified as defined and described herein, to insert a polypeptide (which may be referred to herein as a “D/E polypeptide”) comprising, preferably consisting of, a stretch of consecutive negative amino acids independently selected from aspartic acid and/or glutamic acid. In embodiments, the D/E polypeptide is inserted between any two adjacent amino acid residues of said CMV polypeptide corresponding to any two adjacent amino acid residues between position 75 and position 85 of SEQ ID NO:45. Typically and preferably, the chimeric CMV polypeptide is capable of forming a modified virus-like particle of CMV upon expression by self-assembly. Thus, in a preferred embodiment, said chimeric CMV polypeptide is capable of forming a modified virus-like particle of CMV by self-assembly, typically and preferably by self-assembly upon expression. Preferably, the chimeric CMV polypeptide is a recombinant CMV polypeptide and is capable of forming a virus-like particle of CMV upon expression by self-assembly in E. coli.

In a preferred embodiment, a chimeric CMV polypeptide described herein also comprises a T helper cell epitope. In other words, a CMV polypeptide may be modified to both (i) comprise a T helper cell epitope and (ii) comprise a D/E polypeptide; such a CMV polypeptide having both modifications may be referred to herein as a chimeric M-CMV polypeptide. Typically, chimeric M-CMV polypeptides are capable of forming a virus-like particle of CMV upon expression by self-assembly. Preferably, the chimeric M-CMV polypeptide is a recombinant M-CMV polypeptide and is capable of forming a virus-like particle of CMV upon expression by self-assembly in E. coli.

N-terminal region of the CMV polypeptide: The term “N-terminal region of the CMV polypeptide” as used herein, refers either to the N-terminus of said CMV polypeptide, and in particular to the N-terminus of a coat protein of CMV, or to the region of the N-terminus of said CMV polypeptide or said coat protein of CMV but starting with the second amino acid of the N-terminus of said CMV polypeptide or said coat protein of CMV if said CMV polypeptide or said coat protein comprises a N-terminal methionine residue. Preferably, in M-CMV polypeptides, in case said CMV polypeptide or said coat protein comprises a N-terminal methionine residue, from a practical point of view, the start-codon encoding methionine will usually be deleted from the CMV polypeptide itself and added to the N-terminus of the T helper (Th) cell epitope. Further preferably, one, two or three additional amino acids, preferably one amino acid, may be optionally inserted between the stating methionine and the Th cell epitope for cloning purposes.

Recombinant polypeptide: In the context of the invention the term “recombinant” when used in the context of a polypeptide refers to a polypeptide which is obtained by a process which comprises at least one step of recombinant DNA technology. Typically and preferably, a recombinant polypeptide is produced in a prokaryotic expression system. It is apparent for the artisan that recombinantly produced polypeptides which are expressed in a prokaryotic expression system such as E. coli may comprise an N-terminal methionine residue. The N-terminal methionine residue is typically cleaved off the recombinant polypeptide in the expression host during the maturation of the recombinant polypeptide. However, the cleavage of the N-terminal methionine may be incomplete. Thus, a preparation of a recombinant polypeptide may comprise a mixture of otherwise identical polypeptides with and without an N-terminal methionine residue. Typically and preferably, a preparation of a recombinant polypeptide comprises less than 10%, more preferably less than 5%, and still more preferably less than 1% recombinant polypeptide with an N-terminal methionine residue.

Recombinant modified virus-like particle: In the context of the invention the term “recombinant modified virus-like particle” refers to a modified virus-like particle (VLP) which is obtained by a process which comprises at least one step of recombinant DNA technology.

Mutated amino acid sequence: The term “mutated amino acid sequence” refers to an amino acid sequence which is obtained by introducing a defined set of mutations into an amino acid sequence to be mutated. In the context of the invention, said amino acid sequence to be mutated typically and preferably is an amino acid sequence of a coat protein of CMV. Thus, a mutated amino acid sequence differs from an amino acid sequence of a coat protein of CMV in at least one amino acid residue, wherein said mutated amino acid sequence and said amino acid sequence to be mutated show a sequence identity of at least 90%. Typically and preferably said mutated amino acid sequence and said amino acid sequence to be mutated show a sequence identity of at least 91%, at least 92%, at least 93%, at least 94%, at least 95%, at least 96%, at least 97%, at least 98%, or at least 99%. Preferably, said mutated amino acid sequence and said sequence to be mutated differ in at most 11, 10, 9, 8, 7, 6, 4, 3, 2, or 1 amino acid residues, wherein further preferably said difference is selected from insertion, deletion, and amino acid exchange (“amino acid exchange” may also be referred to as “amino acid substitution” or “amino acid replacement”). Preferably, the mutated amino acid sequence differs from an amino acid sequence of a coat protein of CMV in least one amino acid, wherein preferably said difference is an amino acid exchange.

The terms “corresponding, correspond or corresponds” when used herein to describe the relationship of specific positions of amino acid residue(s) within polypeptides and amino acid sequences, respectively, refers to the position of an amino acid residue(s) within an amino acid sequence, which corresponds to given and specific amino acid residue(s) of another amino acid sequence that can be identified by sequence alignment, typically and preferably by using the BLASTP algorithm, most preferably using the standard settings. Typical and preferred standard settings are: expect threshold: 10; word size: 3; max matches in a query range: 0; matrix: BLOSUM62; gap costs: existence 11, extension 1; compositional adjustments: conditional compositional score matrix adjustment.

Sequence identity: The sequence identity of two given amino acid sequences is determined based on an alignment of both sequences. Algorithms for the determination of sequence identity are available to the artisan. Preferably, the sequence identity of two amino acid sequences is determined using publicly available computer homology programs such as the “BLAST” program (http://blast.ncbi.nlm.nih.gov/Blast.cgi) or the “CLUSTALW” (http://www.genome.jp/tools/clustalw/), and hereby preferably by the “BLAST” program provided on the NCBI homepage at http://blast.ncbi.nlm.nih.gov/Blast.cgi, using the default settings provided therein. Typical and preferred standard settings are: expect threshold: 10; word size: 3; max matches in a query range: 0; matrix: BLOSUM62; gap costs: existence 11, extension 1; compositional adjustments: conditional compositional score matrix adjustment.

Amino acid exchange, replacement, or substitution: The terms “amino acid exchange”, “amino acid replacement”, and “amino acid substitution” interchangeably refer to the exchange, replacement, or substitution, of a given amino acid residue in an amino acid sequence by any other amino acid residue having a different chemical structure, preferably by another proteinogenic amino acid residue. Thus, in contrast to insertion or deletion of an amino acid, the amino acid exchange does not change the total number of amino acids of said amino acid sequence. Amino acid substitutions may be conservative amino acid substitution(s) or may be non-conservative amino acid substitution(s). Conservative substitutions may comprise those that are described by Dayhoff in “The Atlas of Protein Sequence and Structure. Vol. 5”, Natl. Biomedical Research.

The term “isoelectric point” as used herein and abbreviated as pI, refers to the pH at which a molecule carries no net electrical charge or is electrically neutral in the statistical mean. In particular, the term “isoelectric point” is used herein to refer to the pH at which antigens, used in the present invention and which are composed of amino acids, carries no net electrical charge or is electrically neutral in the statistical mean. At a pH below their pI, such antigens carry a net positive charge; above their pI they carry a net negative charge. Typically and preferably when referring to pI values, and in particular to pI values of antigens of the present invention and within the present disclosure, said pI values are determined by entering the primary amino acid sequence for a particular protein and antigen, respectively, into the ExPASy Compute pI/MW tool described by Gasteiger et al (Gasteiger, E., Hoogland, C., Gattiker, A., Duvaud, S., Wilkins, M. R., Appel, R. D., & Bairoch, A., Protein Identification and Analysis Tools on the ExPASy Server, (In) John M. Walker (ed): The Proteomics Protocols Handbook, Humana Press (2005). Thus, if referred herein to the ExPASy Compute pI/MW tool is refers to the one described by Gasteiger et al. The tool calculates the theoretical isoelectric point pI and Mw of a specified Swiss-Prot/TrEMBL entry or a user-entered amino acid sequence. The pI of the protein is calculated using pK values of amino acids described in Bjellqvist et al., which were defined by examining polypeptide migration between pH 4.5 to 7.3 in an immobilised pH gradient gel environment with 9.2M and 9.8M urea at 15° C. or 25° C. (Bjellqvist, B. et al, 1993, Electrophoresis 14:1023-1031; Bjellqvist, B. er al, 1994, Electrophoresis 15:529-539).

Epitope: The term “epitope” refers to continuous or discontinuous portion(s) of a polypeptide or an antigen, wherein said portion(s) can be specifically bound by an antibody or by a T-cell receptor within the context of an MHC molecule. With respect to antibodies, specific binding excludes non-specific binding but does not necessarily exclude cross-reactivity. An epitope typically comprise 5-20 amino acids in a spatial conformation which is unique to the antigenic site.

T helper (Th) cell epitope: The terms “T helper cell epitope” or “Th cell epitope”, as interchangeably used herein, refer to an epitope that is capable of recognition by a helper T cell. Typically and preferably, the term “Th cell epitope” as used herein refers to a Th cell epitope that is capable of binding to at least one, preferably more than one, MHC class II molecules. The simplest way to determine whether a peptide sequence is a Th cell epitope is to measure the ability of the peptide to bind to individual MHC class II molecules. This may be measured by the ability of the peptide to compete with the binding of a known Th cell epitope peptide to the MHC class II molecule. A representative selection of HLA-DR molecules are described in e.g. Alexander J, et al., Immunity (1994) 1:751-761. Affinities of Th cell epitopes for MHC class II molecules should be at least 10-5M. A representative collection of MHC class II molecules present in different individuals is given in Panina-Bordignon P, et al., Eur J Immunol (1989) 19:2237-2242. As a consequence, the term “Th cell epitope” as used herein preferably refers to a Th cell epitope that generates a measurable T cell response upon immunization and boosting. Moreover, and again further preferred, the term “Th cell epitope” as used herein preferably refers to a Th cell epitope that is capable of binding to at least one, preferably to at least two, and even more preferably to at least three DR alleles selected from of DR1, DR2w2b, DR3, DR4w4, DR4w14, DR5, DR7, DR52a, DRw53, DR2w2a; and preferably selected from DR1, DR2w2b, DR4w4, DR4w14, DR5, DR7, DRw53, DR2w2a, with an affinity at least 500 nM (as described in Alexander J, et al., Immunity (1994) 1:751-761 and references cited herein); a preferred binding assay to evaluate said affinities is the one described by Sette A, et al., J Immunol (1989) 142:35-40. In an even again more preferable manner, the term “Th cell epitope” as used herein refers to a Th cell epitope that is capable of binding to at least one, preferably to at least two, and even more preferably to at least three DR alleles selected from DR1, DR2w2b, DR4w4, DR4w14, DR5, DR7, DRw53, DR2w2a, with an affinity at least 500 nM (as described in Alexander J, et al., Immunity (1994) 1:751-761 and references cited herein); a preferred binding assay to evaluate said affinities is the one described by Sette A, et al., J Immunol (1989) 142:35-40. Th cell epitopes are described, and known to the skilled person in the art, such as by Alexander J, et al., Immunity (1994) 1:751-761, Panina-Bordignon P, et al., Eur J Immunol (1989) 19:2237-2242, Calvo-Calle J M, et al., J Immunol (1997) 159:1362-1373, and Valmori D, et al., J Immunol (1992) 149:717-721.

Amino acid linker: The term “amino acid linker” as used herein, refers to a linker consisting exclusively of amino acid residues. The amino acid residues of the amino acid linker are composed of naturally occurring amino acids or unnatural amino acids known in the art, all-L or all-D or mixtures thereof. The amino acid residues of the amino acid linker are preferably naturally occurring amino acids, all-L or all-D or mixtures thereof. In a preferred embodiment, said amino acid linker consists of naturally occurring alpha amino acids, all in its L-configuration.

G-linker: The term “G-linker”, as used herein refers to an amino acid linker solely consisting of glycine amino acid residues. A G-linker in accordance with the present invention preferably comprises at least two glycine residues and at most ten glycine residues.

GS-linker: The term “GS-linker”, as used herein refers to an amino acid linker solely consisting of glycine and serine amino acid residues. A GS-linker in accordance with the present invention preferably comprises at least one glycine and at least one serine residue. Typically and preferably, the GS-linker has a length of at most 30 amino acids.

GS*-linker: The term “GS*-linker”, as used herein refers to an amino acid linker comprising at least one glycine, at least one serine, and at least one amino acid residue selected from Thr, Ala, Lys, and Cys. Typically and preferably, the GS*-linker has a length of at most 30 amino acids.

The term “amino acid”, as used herein, refers to organic compounds containing the functional groups amine (—NH2) and carboxylic acid (—COOH) and its zwitterions, typically and preferably, along with a side chain specific to each amino acid. The term “amino acid” typically and preferably includes amino acids that occur naturally, such as proteinogenic amino acids (produced by RNA-translation), non-proteinogenic amino acids (produced by other metabolic mechanisms, e.g. posttranslational modification), standard or canonical amino acids (that are directly encoded by the codons of the genetic code) and non-standard or non-canonical amino acids (not directly encoded by the genetic code). Naturally occurring amino acids include non-eukaryotic and eukaryotic amino acids. The term “amino acid”, as used herein, also includes unnatural amino acids that are chemically synthesized; alpha-(α-), beta-(β-), gamma-(γ-) and delta-(δ-) etc. amino acids as well as mixtures thereof in any ratio; and, if applicable such as for alpha-(α-) amino acids, any isomeric form of an amino acid, i.e. its D-stereoisomers and L-stereoisomers (alternatively addressed by the (R) and(S) nomenclature) as well as mixtures thereof in any ratio including in a racemic ratio of 1:1. The term “D-stereoisomer”, “L-stereoisomer”, “D-amino acid” or “L-amino acid” refers to the chiral alpha carbon of the amino acids. In a preferred embodiment, the term amino acid refers to an alpha amino acid, preferably to a naturally occurring alpha amino acid, further preferably to a naturally occurring alpha amino acid in its L-configuration.

Associated: The terms “associated” or “association” as used herein refer to all possible ways, preferably chemical interactions, by which two molecules are joined together. Chemical interactions include covalent and non-covalent interactions. Typical examples for non-covalent interactions are ionic interactions, hydrophobic interactions or hydrogen bonds, whereas covalent interactions are based, by way of example, on covalent bonds such as ester, ether, phosphoester, carbon-phosphorus bonds, carbon-sulfur bonds such as thioether, or imide bonds.

Attachment Site, First: As used herein, the phrase “first attachment site” refers to an element which is naturally occurring with the virus-like particle or which is artificially added to the virus-like particle, and to which the second attachment site may be linked. The first attachment site preferably is a protein, a polypeptide, an amino acid, a peptide, a sugar, a polynucleotide, a natural or synthetic polymer, a secondary metabolite or compound such as biotin, fluorescein, retinol, digoxigenin, metal ions, phenylmethylsulfonylfluoride, or a chemically reactive group such as an amino group, a carboxyl group, a sulfhydryl group, a hydroxyl group, a guanidinyl group, histidinyl group, or a combination thereof. A preferred embodiment of a chemically reactive group being the first attachment site is the amino group of an amino acid residue, preferably the amino group of the side chain of a lysine residue. The first attachment site is typically located on the surface, and preferably on the outer surface of the VLP. Multiple first attachment sites are typically present on the surface, preferably on the outer surface, of the VLP, typically in a repetitive configuration. In a preferred embodiment, the first attachment site is associated with the VLP through at least one covalent bond, preferably through at least one peptide bond. In a further preferred embodiment, the first attachment site is naturally occurring with the VLP. Alternatively, in a preferred embodiment, the first attachment site is artificially added to the VLP. In a very preferred embodiment, said first attachment site is the amino group of a lysine residue of the amino acid sequence of said VLP polypeptide.

Attachment Site, Second: As used herein, the phrase “second attachment site” refers to an element which is naturally occurring with or which is artificially added to the antigen and to which the first attachment site may be linked. The second attachment site of the antigen preferably is a protein, a polypeptide, a peptide, an amino acid, a sugar, a polynucleotide, a natural or synthetic polymer, a secondary metabolite or compound such as biotin, fluorescein, retinol, digoxigenin, metal ions, phenylmethylsulfonylfluoride, or a chemically reactive group such as an amino group, a carboxyl group, a sulfhydryl group, a hydroxyl group, a guanidinyl group, histidinyl group, or a combination thereof. A preferred embodiment of a chemically reactive group being the second attachment site is a sulfhydryl group, preferably the sulfhydryl group of a cysteine residue. The term “antigen with at least one second attachment site” refers, therefore, to a construct comprising the antigen and at least one second attachment site. However, in particular for a second attachment site that is not naturally occurring within the antigen, such a construct typically and preferably further comprises a “linker”. In another preferred embodiment the second attachment site is associated with the antigen through at least one covalent bond, preferably through at least one peptide bond. In a further embodiment, the second attachment site is naturally occurring within the antigen. In another further preferred embodiment, the second attachment site is artificially added to the antigen through a linker, wherein said linker preferably comprises or alternatively consists of a cysteine. Preferably, the linker is fused to the antigen by a peptide bond.

Linked: The terms “linked” or “linkage” as used herein, refer to all possible ways, preferably chemical interactions, by which the at least one first attachment site and the at least one second attachment site are joined together. Chemical interactions include covalent and non-covalent interactions. Typical examples for non-covalent interactions are ionic interactions, hydrophobic interactions or hydrogen bonds, whereas covalent interactions are based, by way of example, on covalent bonds such as ester, ether, phosphoester, carbon-phosphorus bonds, carbon-sulfur bonds such as thioether, or imide bonds. In certain preferred embodiments the first attachment site and the second attachment site are linked through at least one covalent bond, preferably through at least one non-peptide bond, and even more preferably through exclusively non-peptide covalent bond(s). The term “linked” as used herein, however, shall not only refer to a direct linkage of the at least one first attachment site and the at least one second attachment site but also, alternatively and preferably, an indirect linkage of the at least one first attachment site and the at least one second attachment site through intermediate molecule(s), and hereby typically and preferably by using at least one, preferably one, heterobifunctional cross-linker. In other embodiments the first attachment site and the second attachment site are linked through at least one covalent bond, preferably through at least one peptide bond, and even more preferably through exclusively peptide bond(s).

Linker: A “linker”, as used herein, either associates the second attachment site with the antigen or already comprises or consists of the second attachment site. Preferably, a “linker”, as used herein, already comprises the second attachment site, typically and preferably as one amino acid residue, preferably as a cysteine residue. A preferred linker is a linker containing at least one amino acid residue. Also preferred is a linker consisting exclusively of amino acid residues. The amino acid residues of the linker are, preferably, composed of naturally occurring amino acids or unnatural amino acids known in the art, all-L or all-D or mixtures thereof. Further preferred embodiments of a linker in accordance with this invention are molecules comprising a sulfhydryl group or a cysteine residue and such molecules are, therefore, also encompassed within this invention. Further linkers useful for the present invention are molecules comprising a C1-6 alkyl-, a cycloalkyl such as a cyclopentyl or cyclohexyl, a cycloalkenyl, aryl or heteroaryl moiety. Moreover, linkers comprising preferably a C1-C6 alkyl-, cycloalkyl-(C5, C6), aryl- or heteroaryl-moiety and additional amino acid(s) can also be used as linkers for the present invention and shall be encompassed within the scope of the invention. Association of the linker with the antigen is preferably by way of at least one covalent bond, more preferably by way of at least one peptide bond.

Antigen: As used herein, the term “antigen” refers to a molecule capable of being bound by an antibody or a T-cell receptor (TCR) if presented by MHC molecule(s). An antigen may additionally be capable of being recognized by the immune system and/or be capable of inducing a humoral immune response and/or be capable of inducing a cellular immune response; the immune response may lead to the activation of B- and/or T-lymphocytes. An antigen can have one or more epitopes (B- and/or T-epitopes). An antigen as used herein may also be mixtures of several individual antigens.

Ordered and repetitive antigen array: As used herein, the term “ordered and repetitive antigen array” refers to a repeating pattern of antigen which typically and preferably is characterized by a high order of uniformity in spatial arrangement of the antigens with respect to the modified VLP of CMV. In one embodiment of the invention, the repeating pattern may be a geometric pattern. Certain embodiments of the invention, such as antigens linked to the modified VLP of CMV, are typical and preferred examples of suitable ordered and repetitive antigen arrays which, moreover, may possess repetitive, preferably strictly repetitive, paracrystalline orders of antigens, preferably with spacing of 1 to 30 nanometers, 1.6 to 30 nanometers, 2 to 15 nanometers, 1.6 to 12 nanometers, 1.6 to 10 nanometers, 2 to 10 nanometers, 1.6 to 8 nanometers, 2 to 8 nanometers, 2 to 7 nanometers, or 1.6 to 7 nanometers.

Coupling efficiency: The coupling efficiency of a virus-like particle with a specific antigen is determined by SDS-PAGE of the coupling reactions. The intensities of Coomassie Blue-stained bands corresponding to components of the coupling reaction are determined by densitometry and used to calculate coupling efficiency. Coupling efficiency is defined as the ratio of (i) the amount of VLP polypeptides coupled to said antigen to (ii) the total amount of VLP polypeptides. Typically and preferably, said coupling efficiency is at least 5%, at least 10%, at least 15%, at least 20%, at least 25%, at least 30%, at least 35%, or at least 40%. Coupling efficiency can also be expressed by the total number of antigens linked to the modified CMV VLP. Coupling efficiency can be dependent on the nature of the antigen, and the total numbers of antigens linked to the modified CMV VLP are typically and preferably at least 5, at least 7, at least 10, at least 15, at least 20, at least 25, at least 30, at least 40, or at least 50 antigens.

Interleukin-1β-D145X mutein antigen: The terms “Interleukin-1β-D145X mutein antigen” or “IL-1β-D145X antigen” or “IL-1β-D145X mutein” or “IL-1β-D145X mutein antigen” as interchangeably used herein and when referring to the antigen of the inventive compositions, refers (i) to a polypeptide comprising, preferably consisting of, the amino acid sequence of interleukin-1β (IL-1β) from any animal species, preferably from a non-human animal, preferably wherein the IL-1β polypeptide is a mature IL-1β polypeptide (e.g., lacking signal peptide(s) and/or propeptide(s)), wherein the amino acid sequence of said IL-1β polypeptides is mutated at the position corresponding to position 145 of reference sequence SEQ ID NO:38, and wherein said mutation is a replacement of the amino acid at position 145 (which amino acid is aspartic acid (D) in reference sequence SEQ ID NO: 38) by an amino acid X, wherein X is preferably selected from the group consisting of lysine (K), tyrosine (Y), phenylalanine (F), asparagine (N), and arginine (R), or (ii) to a polypeptide having a sequence identity of at least 90%, at least 91%, at least 92%, at least 93%, at least 94%, at least 95%, at least 96%, at least 97%, at least 98%, or at least 99% with the amino acid sequence as defined in (i) and, preferably, having an amino acid residue X at the position corresponding to position 145 of reference sequence SEQ ID NO:38 (which amino acid is aspartic acid (D) in SEQ ID NO: 38), wherein X is preferably selected from the group consisting of lysine (K), tyrosine (Y), phenylalanine (F), asparagine (N), and arginine (R). An IL-1β-D145X mutein may be said to be “derived from” a species when the IL-1β portion of the IL-1β-D145X mutein is at least 91%, at least 92%, at least 93%, at least 94%, at least 95%, at least 96%, at least 97%, at least 98%, or at least 99% identical to the amino acid sequence of the wild type IL-1β polypeptide or the mature wild type IL-1β polypeptide of that species. An IL-1β-D145X mutein typically and preferably comprises reduced, preferably eliminated, natural biological activity compared to the natural biological activity of corresponding wild type IL-1β polypeptide, preferably in vivo and/or in vitro, preferably as assessed using a cell proliferation assay, as demonstrated by the Examples. For example, a feline IL-1β-D145X mutein (such as for example a feline IL-1β-D145K mutein) preferably has reduced, or preferably eliminated, natural biological activity as compared to its wild-type feline IL-1β counterpart polypeptide (SEQ ID NO:38). As another example a canine IL-1β-D145X mutein (such as for example a canine IL-1β-D145K mutein) preferably has reduced, or preferably eliminated, natural biological activity as compared to its wild-type canine IL-1β counterpart polypeptide (SEQ ID NO:29). “Counterpart” refers to the same species, and, preferably, the same portion of the polypeptide.

Herein, “natural biological activity” refers to the biological functions performed by wild type IL-1β, such as, e.g., its pro-inflammatory activities. “Natural biological activity” does not, herein, refer to antigenicity. Accordingly, while an IL-1β-D145X mutein preferably comprises less or reduced natural biological activity than wild type IL-1β (and may preferably be inactive for natural biological activit(ies)), an IL-1β-D145X antigen is preferably capable of inducing production of neutralizing anti-wild type IL-1β and/or anti-IL-1β-D145X antibodies; preferably more capable of inducing production of neutralizing anti-wild type IL-1β and/or anti-IL-1β-D145X antibodies than is wild type IL-1β. Moreover, an IL-1β-D145X antigen is typically and preferably capable of inducing production anti-IL-1β-D145X and/or anti-wild type IL-1β antibodies in an animal, when administered to said animal in form of the inventive compositions (e.g., when conjugated to CMV VLPs, particularly the inventive CMV VLPs). Preferably, an IL-1β-D145X antigen conjugated to CMV VLPs, particularly the inventive CMV VLPs, is more capable of inducing production of neutralizing anti-wild type IL-1β and/or anti-IL-1β-D145X antibodies than is wild type IL-1β. Preferably, said anti IL-1β antibodies and said IL-1β-D145X antibodies are capable of neutralizing one or more natural biological activity of IL-1β in an in vitro assay and/or in an in vivo assay, such as described herein. For example, an IL-1β-D145K antigen is typically and preferably capable of inducing production of anti-IL-1β-D145K and/or anti-IL-1β antibodies in an animal, particularly when administered to said animal in form of the inventive compositions (e.g., when conjugated to CMV VLPs, particularly the inventive modified CMV VLPs). Preferably, said anti IL-1β antibodies and/or said IL-1β-D145K antibodies are capable of neutralizing the natural biological activity of IL-1β in an in vitro assay and/or in an in vivo assay. Compositions comprising IL-1β-D145X muteins may be administered to an animal of the same animal species from which the IL-1β-D145X mutein is derived, or to an animal of a different species.

IL-1β-D145X antigens from various animal species are contemplated. Preferred animal species include non-human mammals, such as companion animals (such as equines) and agriculturally significant animals (such as equines, bovines, ovines, caprines, and porcines). Accordingly, equine IL-1β-D145X (eIL-1β-D145X), bovine IL-1β-D145X (bIL-1β-D145X), ovine IL-1β-D145X (bIL-1β-D145X), and porcine IL-1β-D145X (pIL-1β-D145X), are contemplated. In a preferred embodiment of the present invention, said interleukin 1β-D145X mutein (IL-1β-D145X mutein) antigen, is an interleukin 1β-D145K mutein (IL-1β-D145K mutein) antigen. Thus, in a preferred embodiment of the present invention, the antigen is an interleukin 1β-D145K mutein (IL-1β-D145K mutein) antigen.

Preferred IL-1β-D145K antigens from various animal species include for example equine IL-1β-D145K (eIL-1β-D145K), bovine IL-1β-D145K (bIL-1β-D145K), ovine IL-1β-D145K (bIL-1β-D145K), caprine IL-1β-D145K (bIL-1β-D145K), and porcine IL-1β-D145K (pIL-1β-D145K), comprising the amino acid lysine (K) at the position corresponding to position 145 of reference sequence SEQ ID NO:38 (which amino acid is aspartic acid (D) in reference sequence SEQ ID NO: 38). However, in other embodiments, the amino acid at the position corresponding to position 145 of reference sequence SEQ ID NO: 38 may, instead of lysine (K), be selected from the group consisting of tyrosine (Y), phenylalanine (F), asparagine (N), and arginine I at the position corresponding to position 145 of reference sequence SEQ ID NO:38.

Adjuvant: The term “adjuvant” as used herein refers to stimulators of the immune response and/or substances that typically allow generation of a depot in the host which when combined with the composition, vaccine or pharmaceutical composition, respectively, of the present invention may provide for a more enhanced immune response. Adjuvants of varying types with different mechanisms of action are described and preferably are able to enhance the antigen-specific antibody response (Pulendran B et al, 2021, Nature Reviews Drug Discovery 20:454-475). Typical and preferred adjuvants include mineral salts (e.g. Aluminum Hydroxide, Aluminum Phosphate), microcrystalline tyrosine, emulsions, microparticles, saponins (Quil A), cytokines, immune potentiators, microbial components/products, liposomes, complexes, and mucosal adjuvants which are known and as described such, and for example, in the Adjuvant Compendium NIAID and VAC (nih.gov) or by Aguilar et al, (Aguilar J C et al, 2007, Vaccine 25:3752-3762), Gerdts (Gerdts V, 2015, Berliner und Münchener Tierärztliche Wochenschrift 128:456-463) and Pasquale et al. (Pasquale et al. 2015, Vaccines 3:320-343). The term “adjuvant” as used herein may also comprise mixtures of adjuvants. Virus-like particles have sometimes been described as an adjuvant. However, the term “adjuvant”, as used within the context of this application, refers to an adjuvant not being the inventive modified virus-like particle. Rather “adjuvant” relates to an additional, distinct component of the inventive compositions, vaccines or pharmaceutical compositions.

Immunostimulatory substance: As used herein, the term “immunostimulatory substance” refers to a substance capable of inducing and/or enhancing an immune response. Immunostimulatory substances, as used herein, include, but are not limited to, toll-like receptor activating substances and substances inducing cytokine secretion. Toll-like receptor activating substances include, but are not limited to, immunostimulatory nucleic acids, peptideoglycans, lipopolysaccharides, lipoteichonic acids, imidazoquinoline compounds, flagellins, lipoproteins, and immunostimulatory organic substances such as taxol.

Immunostimulatory nucleic acid (ISS-NA): As used herein, the term “immunostimulatory nucleic acid” refers to a nucleic acid capable of inducing and/or enhancing an immune response. Immunostimulatory nucleic acids comprise ribonucleic acids and/or deoxyribonucleic acids, wherein both, ribonucleic acids and deoxyribonucleic acids may be either double stranded or single stranded. Preferred ISS-NA are deoxyribonucleic acids, wherein further preferably said deoxyribonucleic acids are single stranded. Preferably, immunostimulatory nucleic acids contain at least one CpG motif comprising an unmethylated C. Very preferred immunostimulatory nucleic acids comprise at least one CpG motif, wherein said at least one CpG motif comprises or preferably consist of at least one, preferably one, CG dinucleotide, wherein the C is unmethylated. Preferably, but not necessarily, said CG dinucleotide is part of a palindromic sequence. The term immunostimulatory nucleic acid also refers to nucleic acids that contain modified bases, preferably 4-bromo-cytosine. Specifically preferred in the context of the invention are ISS-NA which are capable of stimulating IFN-alpha production in dendritic cells. Immunostimulatory nucleic acids useful for the purpose of the invention are described, for example, in WO2007/068747 and U.S. Pat. No. 8,541,559.

Oligonucleotide: As used herein, the term “oligonucleotide” refers to a nucleic acid sequence comprising two or more nucleotides, preferably about 6 to about 200 nucleotides, and more preferably 20 to about 100 nucleotides, and most preferably 20 to 40 nucleotides. Oligonucleotides are polyribonucleotides or polydeoxribonucleotides and are preferably selected from (a) unmodified RNA or DNA, and (b) modified RNA or DNA. The modification may comprise the backbone or nucleotide analogues. Oligonucleotides are preferably selected from the group consisting of (a) single- and double-stranded DNA, (b) DNA that is a mixture of single- and double-stranded regions, (c) single- and double-stranded RNA, (d) RNA that is mixture of single- and double-stranded regions, and (e) hybrid molecules comprising DNA and RNA that are single-stranded or, more preferably, double-stranded or a mixture of single- and double-stranded regions. Preferred nucleotide modifications/analogs are selected from the group consisting of (a) peptide nucleic acid, (b) inosin, (c) tritylated bases, (d) phosphorothioates, (e) alkylphosphorothioates, (f) 5-nitroindole desoxyribofliranosyl, (g) 5-methyldesoxycytosine, and (h) 5,6-dihydro-5,6-dihydroxydesoxythymidine. Phosphorothioated nucleotides are protected against degradation in a cell or an organism and are therefore preferred nucleotide modifications. Unmodified oligonucleotides consisting exclusively of phosphodiester bound nucleotides, typically are more active than modified nucleotides and are therefore generally preferred in the context of the invention. Most preferred are oligonucleotides consisting exclusively of phosphodiester bound oligonucleotides, wherein further preferably said oligonucleotides are single stranded. Further preferred are oligonucleotides capable of stimulating IFN-alpha production in cells, preferably in dendritic cells. Very preferred oligonucleotides capable of stimulating IFN-alpha production in cells are selected from A-type CpGs and C-type CpGs. Further preferred are RNA-molecules without a Cap.

CpG motif: As used herein, the term “CpG motif” refers to a pattern of nucleotides that includes an unmethylated central CpG, i.e. the unmethylated CpG dinucleotide, in which the C is unmethylated, surrounded by at least one base, preferably one or two nucleotides, flanking (on the 3′ and the 5′ side of) the central CpG. Typically and preferably, the CpG motif as used herein, comprises or alternatively consists of the unmethylated CpG dinucleotide and two nucleotides on its 5′ and 3′ ends. Without being bound by theory, the bases flanking the CpG confer a significant part of the activity to the CpG oligonucleotide.

Unmethylated CpG-containing oligonucleotide: As used herein, the term “unmethylated CpG-containing oligonucleotide” or “CpG” refers to an oligonucleotide, preferably to an oligodeoxynucleotide, containing at least one CpG motif. Thus, a CpG contains at least one unmethylated cytosine, guanine dinucleotide. Preferred CpGs stimulate/activate, e.g. have a mitogenic effect on, or induce or increase cytokine expression by, a vertebrate bone marrow derived cell. For example, CpGs can be useful in activating B cells, NK cells and antigen-presenting cells, such as dendritic cells, monocytes and macrophages. Preferably, CpG relates to an oligodeoxynucleotide, preferably to a single stranded oligodeoxynucleotide, containing an unmethylated cytosine followed 3′ by a guanosine, wherein said unmethylated cytosine and said guanosine are linked by a phosphate bond, wherein preferably said phosphate bound is a phosphodiester bound or a phosphorothioate bound, and wherein further preferably said phosphate bond is a phosphodiester bound. CpGs can include nucleotide analogs such as analogs containing phosphorothioester bonds and can be double-stranded or single-stranded. Generally, double-stranded molecules are more stable in vivo, while single-stranded molecules have increased immune activity. Preferably, as used herein, a CpG is an oligonucleotide that is at least about ten nucleotides in length and comprises at least one CpG motif, wherein further preferably said CpG is 10 to 60, more preferably 15 to 50, still more preferably 20 to 40, still more preferably about 30, and most preferably exactly 30 nucleotides in length. A CpG may consist of methylated and/or unmethylated nucleotides, wherein said at least one CpG motif comprises at least one CG dinucleotide wherein the C is unmethylated. The CpG may also comprise methylated and unmethylated sequence stretches, wherein said at least one CpG motif comprises at least one CG dinucleotide wherein the C is unmethylated. Very preferably, CpG relates to a single stranded oligodeoxynucleotide containing an unmethylated cytosine followed 3′ by a guanosine, wherein said unmethylated cytosine and said guanosine are linked by a phosphodiester bound. The CpGs can include nucleotide analogs such as analogs containing phosphorothioester bonds and can be double-stranded or single-stranded. Generally, phosphodiester CpGs are A-type CpGs as indicated below, while phosphothioester stabilized CpGs are B-type CpGs. Preferred CpG oligonucleotides in the context of the invention are A-type CpGs.

A-type CpG: As used herein, the term “A-type CpG” or “D-type CpG” refers to an oligodeoxynucleotide (ODN) comprising at least one CpG motif. A-type CpGs preferentially stimulate activation of T cells and the maturation of dendritic cells and are capable of stimulating IFN-alpha production. In A-type CpGs, the nucleotides of the at least one CpG motif are linked by at least one phosphodiester bond. A-type CpGs comprise at least one phosphodiester bond CpG motif which may be flanked at its 5′ end and/or, preferably and, at its 3′ end by phosphorothioate bound nucleotides. Preferably, the CpG motif, and hereby preferably the CG dinucleotide and its immediate flanking regions comprising at least one, preferably two nucleotides, are composed of phosphodiester nucleotides. Preferred A-type CpGs exclusively consist of phosphodiester (PO) bond nucleotides. Typically and preferably, the poly G motif comprises or alternatively consists of at least one, preferably at least three, at least 4, 5, 6, 7, 8, 9, 10, 11, 12, 13, 14, or 15 G's (guanosines), most preferably by at least 10 G's. Preferably, the A-type CpG of the invention comprises or alternatively consists of a palindromic sequence.

Packaged: The term “packaged” as used herein refers to the state of a polyanionic macromolecule and/or immunostimulatory substance in relation to the core particle and VLP, respectively. The term “packaged” as used herein includes binding that may be covalent, e.g., by chemically coupling, or non-covalent, e.g., ionic interactions, hydrophobic interactions, hydrogen bonds, etc. The term also includes the enclosement, or partial enclosement, of a polyanionic macromolecule and/or immunostimulatory substance. Thus, the polyanionic macromolecule or immunostimulatory substances can be enclosed by the VLP without the existence of an actual binding, in particular of a covalent binding. In preferred embodiments utilizing at least one polyanionic macromolecule and/or immunostimulatory substances, the at least one polyanionic macromolecule and/or immunostimulatory substances is packaged inside the VLP, most preferably in a non-covalent manner. In case said immunostimulatory substances is nucleic acid, preferably a DNA, the term packaged implies that said nucleic acid is not accessible to nucleases hydrolysis, preferably not accessible to DNAse hydrolysis (e.g. DNaseI or Benzonase), wherein preferably said accessibility is assayed as described in Examples 11-17 of WO2003/024481A2 (U.S. Pat. No. 8,691,209).

Effective amount: As used herein, the term “effective amount” refers to an amount necessary and/or sufficient to realize a desired biologic effect. In embodiments, a desired. biological effect may include beneficial and/or desired clinical results. In an embodiment, the beneficial and/or desired clinical results may be measurable via qualitative and/or quantitative measures. An effective amount of a composition, and/or a pharmaceutical composition, would be the amount that achieves the desired biological effect, and such an amount can be determined as a matter of routine by a person skilled in the art. The effective amount can vary depending on such factors as the particular composition being administered, the desired biological effect, the age of the subject, and/or the size of the subject. One of ordinary skill in the art can empirically determine the effective amount of a particular composition of the present invention without necessitating undue experimentation.

Animal: The term “animal”, as used herein, include, but is not limited to, a vertebrate animal, a mammal, a rodent (e.g., a guinea pig, a hamster), a bovine (e.g., a cow), a porcine (e.g., a pig), an ovine (e.g., a sheep), a caprine (e.g., a goat), an equine (e.g., a horse), or a primate. Preferably, the subject is a non-human animal, preferably a non-human mammal (such as, e.g., a horse, a sheep, cattle, a goat, or a pig). In a preferred embodiment said subject is a non-human mammal selected from a horse, a sheep, cow, sheep, goat, or a pig.

Veterinary composition: As used herein, the term “veterinary composition” refers to a composition suitable for use in non-human animals.

Administration: Administration may be by any route suitable to achieve the beneficial and/or desired clinical results. Appropriate routes of administration may be influenced or dictated by such factors as the properties of the composition administered, by the desired clinical results, and by the avoidance of adverse effects. Appropriate routes of administration may be determined by those of skill in the art without undue experimentation. For example, one of skill in the art realizes that a composition subject to degradation by acids is not optimally delivered orally, unless protected by, e.g., a coating. In embodiments, administration may by via infusion and/or injection. In embodiments, injection may be intravenous, intramuscular, and/or subcutaneous.

Dosing. Appropriate dosing may be influenced or dictated by such factors as the properties of the composition administered, by the desired clinical results, and by the avoidance of adverse effects. Appropriate timing, number, and amount of dose(s) may be determined by those of skill in the art without undue experimentation. Administration may be in one or more than one dose. If in more than one does, the amount of each does and/or the site and/or route of administration may vary between doses.

Reduction of adverse effects: Administration of compositions commonly may result in, cause, and/or be accompanied by adverse effects. In preferred embodiments, adverse effects that result from, are caused by, and/or accompany administration of an effective amount of an inventive composition described herein are reduced in occurrence, duration, and/or severity as compared to those adverse effects that result from, are caused by, and/or accompany administration of an effective amount of non-inventive composition; e.g., one not described and/or claimed herein. In embodiments, the occurrence, duration, and/or severity of adverse events may be measurable via qualitative and/or quantitative measures. In embodiments, adverse events may include localized and/or systemic adverse effects. As non-limiting examples localized adverse effects may include one or more of those set forth as rows 1-8 of Table D. As non-limiting examples, systemic adverse effects may include one or more of pyrexia and/one or more of those set forth as rows B-J of Table E.

In a first aspect, the present invention provides a composition, preferably a veterinary composition, comprising
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:45; and
   (ii) optionally, a D/E polypeptide comprising, preferably consisting of, a stretch of consecutive negative amino acids, wherein said negative amino acids are independently selected from aspartic acid and/or glutamic acid, wherein said D/E polypeptide is inserted between any two adjacent amino acid residues of said CMV polypeptide corresponding to any two adjacent amino acid residues between position 75 and position 85 of SEQ ID NO:45.
(b) at least one antigen, wherein said antigen comprises at least one second attachment site, and wherein said antigen is an interleukin 1β D145X mutein (IL-1β-D145X mutein) antigen, preferably an interleukin 1β D145K mutein (IL-1β-D145K mutein) antigen; and

wherein (a) and (b) are linked through said at least one first and said at least one second attachment site, preferably via at least one covalent non-peptide bond.

In a further aspect, the present invention provides a composition, preferably a veterinary composition, comprising
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:45; and
   (ii) a D/E polypeptide comprising, preferably consisting of, a stretch of consecutive negative amino acids, wherein said negative amino acids are independently selected from aspartic acid and/or glutamic acid, wherein said D/E polypeptide is inserted between any two adjacent amino acid residues of said CMV polypeptide corresponding to any two adjacent amino acid residues between position 75 and position 85 of SEQ ID NO:45.
(b) at least one antigen, wherein said antigen comprises at least one second attachment site, and wherein said antigen is an interleukin 1β D145X mutein (IL-1β-D145X mutein) antigen, preferably an interleukin 1β D145K mutein (IL-1β-D145K mutein) antigen; and
   wherein (a) and (b) are linked through said at least one first and said at least one second attachment site, preferably via at least one covalent non-peptide bond.

Thus, in a further aspect, the present invention provides a composition comprising
(a) a modified VLP of CMV comprising at least one first attachment site;
(b) at least one interleukin 1β D145X mutein (IL-1β-D145X mutein) antigen, preferably at least one interleukin 1β D145K mutein (IL-1β-D145K mutein) antigen, wherein said antigen comprises at least one second attachment site;

wherein said modified VLP of CMV comprises at least one chimeric CMV polypeptide, and wherein said at least one chimeric CMV polypeptide comprises, preferably consists of,
(i) a CMV polypeptide, wherein said CMV polypeptide comprises a coat protein of CMV or an amino acid sequence having a sequence identity of at least 75% with SEQ ID NO:45; and
(ii) a D/E polypeptide comprising, preferably consisting of, a stretch of consecutive negative amino acids, wherein said negative amino acids are independently selected from aspartic acid and/or glutamic acid, and wherein said D/E polypeptide is inserted between any two adjacent amino acid residues of said CMV polypeptide corresponding to any two adjacent amino acids residue between position 75 and position 85 of SEQ ID NO:45; and
   wherein (a) and (b) are linked through said at least one first and said at least one second attachment site, preferably via at least one covalent non-peptide bond.

In a preferred embodiment, said chimeric CMV polypeptide further comprises a T helper cell epitope, wherein preferably said T helper cell epitope replaces an N-terminal region of said CMV polypeptide, and wherein further preferably said N-terminal region of said CMV polypeptide corresponds to amino acids 2-12 of SEQ ID NO:45, and wherein again further preferably said T helper cell epitope is derived from tetanus toxin or is a PADRE sequence, wherein very preferably, said Th cell epitope comprises, again further preferably consists of, the amino acid sequence of SEQ ID NO:47 or SEQ ID NO:48. In a further very preferred embodiment, said CMV polypeptide is a coat protein of CMV or an amino acid sequence having a sequence identity of at least 90%, at least 92%, at least 95%, or at least 98% with SEQ ID NO:45.

Thus, in another aspect, the present invention provides a composition, preferably a veterinary composition, comprising
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:45; and
   (ii) a D/E polypeptide comprising, preferably consisting of, a stretch of consecutive negative amino acids, wherein said negative amino acids are independently selected from aspartic acid and/or glutamic acid, wherein said D/E polypeptide is inserted between any two adjacent amino acid residues of said CMV polypeptide corresponding to any two adjacent amino acid residues between position 75 and position 85 of SEQ ID NO: 45 and
   (iii) a T helper cell epitope, wherein said T helper cell epitope replaces a N-terminal region of said CMV polypeptide; and
(b) at least one antigen, wherein said antigen comprises at least one second attachment site, and wherein said antigen is an interleukin 1β D145X mutein (IL-1β-D145X mutein) antigen, preferably an interleukin 1β D145K mutein (IL-1β-D145K mutein) antigen; and
   wherein (a) and (b) are linked through said at least one first and said at least one second attachment site via, preferably at least one covalent non-peptide bond.

In a further very preferred embodiment, said stretch of consecutive negative amino acids comprises, preferably consists of SEQ ID NO:1 or SEQ ID NO:2.

Thus, in a further aspect, the present invention provides a composition, preferably a veterinary composition, comprising
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:45, wherein preferably said CMV polypeptide is a coat protein of CMV or an amino acid sequence having a sequence identity of at least 90%, preferably 95%, with SEQ ID NO:45; and
   (ii) a D/E polypeptide comprising, preferably consisting of, a stretch of consecutive negative amino acids, wherein said negative amino acids are independently selected from aspartic acid and/or glutamic acid, and wherein said D/E polypeptide is inserted between any two adjacent amino acid residues of said CMV polypeptide corresponding to any two adjacent amino acid residues between position 75 and position 85 of SEQ ID NO:45, and wherein said stretch of consecutive negative amino acids comprises, preferably consists of, SEQ ID NO:1 or SEQ ID NO:2;
(b) at least one antigen, wherein said antigen comprises at least one second attachment site, and wherein said antigen is an interleukin 1β D145X mutein (IL-1β-D145X mutein) antigen, preferably an interleukin 1β D145K mutein (IL-1β-D145K mutein) antigen; and

wherein (a) and (b) are linked through said at least one first and said at least one second attachment site, preferably via at least one covalent non-peptide bond.

Thus, in another aspect, the present invention provides a composition, preferably a veterinary composition, comprising
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:45, wherein preferably said CMV polypeptide is a coat protein of CMV or an amino acid sequence having a sequence identity of at least 90%, preferably 95% with SEQ ID NO:45;
   (ii) a D/E polypeptide comprising, preferably consisting of, a stretch of consecutive negative amino acids, wherein said negative amino acids are independently selected from aspartic acid and/or glutamic acid, and wherein said D/E polypeptide is inserted between any two consecutive amino acid residues of said CMV polypeptide corresponding to any two consecutive amino acid residues between position 75 and position 85 of SEQ ID NO:45, and wherein said stretch of consecutive negative amino acids comprises, preferably consists of SEQ ID NO:1 or SEQ ID NO:2; and
   (iii) a T helper cell epitope, wherein said T helper cell epitope replaces a N-terminal region of said CMV polypeptide; and
(b) at least one antigen, wherein said antigen comprises at least one second attachment site, and wherein said antigen is an interleukin 1β D145X mutein (IL-1β-D145X mutein) antigen, preferably an interleukin 1β D145K mutein (IL-1β-D145K mutein) antigen; and

wherein (a) and (b) are linked through said at least one first and said at least one second attachment site, preferably via at least one covalent non-peptide bond.

In a preferred embodiment, a D/E polypeptide comprising said stretch of consecutive negative amino acids further comprises a first amino acid linker and/or a second amino acid linker, wherein said first amino acid linker is positioned at the N-terminus of said stretch of consecutive negative amino acids, and said second amino acid linker is positioned at the C-terminus of said stretch of consecutive negative amino acids, and wherein said first and said second amino acid linker are independently selected from the group consisting of (a.) a polyglycine linker (G-linker) having an amino acid sequence (Gly)ₙ of a length of n=2-10; (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and (c.) an amino acid linker (GS*-linker) comprising at least one Gly, at least one Ser, and at least one amino acid selected from Thr, Ala, Lys, and Cys. In a preferred embodiment, said D/E polypeptide comprising said stretch of consecutive negative amino acids further comprises a first amino acid linker and a second amino acid linker, wherein said first amino acid linker is positioned at the N-terminus of said stretch of consecutive negative amino acids, and said second amino acid linker is positioned at the C-terminus of said stretch of consecutive negative amino acids, and wherein said first and said second amino acid linker are independently selected from a glycine-serine linker (GS-linker) comprising at least one glycine and at least one serine, wherein said GS linker has an amino acid sequence of (GS)ᵣ(GₛS)ₜ(GS)ᵤ with r=0 or 1, s=1-5, t=1-5 and u=0 or 1 or an amino acid linker (GS*-linker) comprising at least one Gly, at least one Ser, and at least one amino acid selected from Thr, Ala, Lys, and Cys. In a further very preferred embodiment, said first amino acid linker comprises, preferably consists of, SEQ ID NO:8. In a further very preferred embodiment, said second amino acid linker comprises, preferably consists of, SEQ ID NO:4 or SEQ ID NO:9. In a further very preferred embodiment, said D/E polypeptide comprises SEQ ID NO:49, SEQ ID NO:50 or SEQ ID NO:51. In a further very preferred embodiment, said D/E polypeptide consists of SEQ ID NO:49, SEQ ID NO: 50 or SEQ ID NO:51.

In a further very preferred embodiment, said D/E polypeptide comprising, preferably consisting of, a stretch of consecutive negative amino acids is inserted between amino acid residues of said CMV polypeptide corresponding to amino acid residues of position 84 and position 85 of SEQ ID NO:45.

Thus, in a further aspect, the present invention provides a composition, preferably a veterinary composition, comprising
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:45, wherein preferably said CMV polypeptide is a coat protein of CMV or an amino acid sequence having a sequence identity of at least 90%, preferably 95% with SEQ ID NO:45; and
   (ii) a D/E polypeptide comprising, preferably consisting of, a stretch of consecutive negative amino acids, wherein said negative amino acids are independently selected from aspartic acid and/or glutamic acid, and wherein said D/E polypeptide is inserted between any two consecutive amino acid residues of said CMV polypeptide corresponding to any two consecutive amino acid residues between position 75 and position 85 of SEQ ID NO:45, and wherein said D/E polypeptide comprises, preferably consists of, SEQ ID NO: 49, SEQ ID NO:50, or SEQ ID NO:51, and wherein preferably said D/E polypeptide is inserted between amino acid residues of said CMV polypeptide corresponding to amino acid residues of position 84 and position 85 of SEQ ID NO:45; and
(b) at least one antigen, wherein said antigen comprises at least one second attachment site, and wherein said antigen is an interleukin 1β D145X mutein (IL-1β-D145X mutein) antigen, preferably an interleukin 1β D145K mutein (IL-1β-D145K mutein) antigen; and

wherein (a) and (b) are linked through said at least one first and said at least one second attachment site, preferably via at least one covalent non-peptide bond.

Thus, in another aspect, the present invention provides a composition, preferably a veterinary composition, comprising
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:45, wherein preferably said CMV polypeptide is a coat protein of CMV or an amino acid sequence having a sequence identity of at least 90%, preferably 95% with SEQ ID NO:45;
   (ii) a D/E polypeptide comprising, preferably consisting of, a stretch of consecutive negative amino acids, wherein said negative amino acids are independently selected from aspartic acid and/or glutamic acid, and wherein said D/E polypeptide is inserted between any two consecutive amino acid residues of said CMV polypeptide corresponding to any two consecutive amino acid residues between position 75 and position 85 of SEQ ID NO:45, and wherein said D/E polypeptide comprises, preferably consists of, SEQ ID NO: 49, SEQ ID NO:50 or SEQ ID NO:51; and wherein preferably said D/E polypeptide is inserted between amino acid residues of said CMV polypeptide corresponding to amino acid residues of position 84 and position 85 of SEQ ID NO:45;
   (iii) a T helper cell epitope, wherein said T helper cell epitope replaces a N-terminal region of said CMV polypeptide; and
(b) at least one antigen, wherein said antigen comprises at least one second attachment site, and wherein said antigen is an interleukin 1β D145X mutein (IL-1β-D145X mutein) antigen, preferably an interleukin 1β D145K mutein (IL-1β-D145K mutein) antigen; and

wherein (a) and (b) are linked through said at least one first and said at least one second attachment site, preferably via at least one covalent non-peptide bond.

In a further very preferred embodiment, said CMV polypeptide comprises, preferably consists of, the amino acid sequence of SEQ ID NO:5, SEQ ID NO:45 or SEQ ID NO: 52, wherein said D/E polypeptide comprising said stretch of consecutive negative amino acids is inserted between amino acid residues of position 88 and position 89 of said SEQ ID NO:5, between amino acid residues of position 84 and position 85 of SEQ ID NO: 45, or between amino acid residues of position 86 and position 87 of SEQ ID NO:52.

Thus, in a further aspect, the present invention provides a composition, preferably a veterinary composition, comprising
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:45;
   (ii) a D/E polypeptide comprising a stretch of consecutive negative amino acids, wherein said negative amino acids are independently selected from aspartic acid and/or glutamic acid, and wherein said D/E polypeptide is inserted between any two consecutive amino acid residues of said CMV polypeptide corresponding to any two consecutive amino acid residues between position 75 and position 85 of SEQ ID NO:45; and
   wherein said CMV polypeptide comprises, preferably consists of, the amino acid sequence of SEQ ID NO:5, SEQ ID NO:45 or SEQ ID NO:52, and wherein said D/E polypeptide comprising said stretch of consecutive negative amino acids is inserted between amino acid residues of position 88 and position 89 of said SEQ ID NO: 5, between amino acid residues of position 84 and position 85 of SEQ ID NO: 45, or between amino acid residues of position 86 and position 87 of SEQ ID NO: 52; and
(b) at least one antigen, wherein said antigen comprises at least one second attachment site, and wherein said antigen is an interleukin 1β D145X mutein (IL-1β-D145X mutein) antigen, preferably an interleukin 1β D145K mutein (IL-1β-D145K mutein) antigen; and
wherein (a) and (b) are linked through said at least one first and said at least one second attachment site, preferably via at least one covalent non-peptide bond.

Thus, in a further aspect, the present invention provides a composition, preferably a veterinary composition, comprising
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:45;
   (ii) a D/E polypeptide comprising a stretch of consecutive negative amino acids, wherein said negative amino acids are independently selected from aspartic acid and/or glutamic acid, and wherein said D/E polypeptide is inserted between any two adjacent amino acid residues of said CMV polypeptide corresponding to any two adjacent amino acid residues between position 75 and position 85 of SEQ ID NO:45;
   (iii) a T helper cell epitope, wherein said T helper cell epitope replaces a N-terminal region of said CMV polypeptide; and
   wherein said CMV polypeptide comprises, preferably consists of, the amino acid sequence of SEQ ID NO:5, SEQ ID NO:45 or SEQ ID NO:52, and wherein said D/E polypeptide comprising said stretch of consecutive negative amino acids is inserted between amino acid residues of position 88 and position 89 of said SEQ ID NO: 5, between amino acid residues of position 84 and position 85 of SEQ ID NO: 45, or between amino acid residues of position 86 and position 87 of SEQ ID NO: 52; and
(b) at least one antigen, wherein said antigen comprises at least one second attachment site, and wherein said antigen is an interleukin 1β D145X mutein (IL-1β-D145X mutein) antigen, preferably an interleukin 1β D145K mutein (IL-1β-D145K mutein) antigen; and

wherein (a) and (b) are linked through said at least one first and said at least one second attachment site, preferably via at least one covalent non-peptide bond.

The herein described and disclosed embodiments, preferred embodiments and very preferred embodiments should apply to all aspects and other embodiments, preferred embodiments and very preferred embodiments irrespective of whether is specifically again referred to or its repetition is avoided for the sake of conciseness.

In a preferred embodiment, said CMV polypeptide comprises, preferably consists of, an amino acid sequence of a coat protein of CMV or a mutated amino acid sequence, wherein said mutated amino acid sequence and said coat protein of CMV show a sequence identity of at least about 90%, preferably of at least about 91%, at least about 92%, at least about 93%, at least about 94% or at least about 95%, further preferably of at least about 96%, at least about 97% or at least about 98%, and again more preferably of at least about 99%; wherein preferably said mutated amino acid sequence and said amino acid sequence to be mutated differ in least one and in at most 11, 10, 9, 8, 7, 6, 5, 4, 3, or 2 amino acid residues, and wherein further preferably these differences are selected from (i) insertion, (ii) deletion, (iii) amino acid exchange (“amino acid exchange” may also be referred to as “amino acid substitution” or “amino acid replacement”), and (iv) any combination of (i) to (iii).

In a preferred embodiment, said CMV polypeptide comprises a coat protein of CMV or an amino acid sequence having a sequence identity of at least 75% with SEQ ID NO: 45. In another preferred embodiment, said CMV polypeptide comprises a coat protein of CMV or an amino acid sequence having a sequence identity of at least 80% with SEQ ID NO: 45. In another preferred embodiment, said CMV polypeptide comprises a coat protein of CMV or an amino acid sequence having a sequence identity of at least 85% with SEQ ID NO: 45. In another preferred embodiment, said CMV polypeptide comprises a coat protein of CMV or an amino acid sequence having a sequence identity of at least 90% with SEQ ID NO: 45.

In another preferred embodiment, said CMV polypeptide comprises a coat protein of CMV or an amino acid sequence having a sequence identity of at least 92% with SEQ ID NO:45. In another preferred embodiment, said CMV polypeptide comprises a coat protein of CMV or an amino acid sequence having a sequence identity of at least 93% with SEQ ID NO:45. In another preferred embodiment, said CMV polypeptide comprises a coat protein of CMV or an amino acid sequence having a sequence identity of at least 95% with SEQ ID NO:45.

In another preferred embodiment, said CMV polypeptide comprises a coat protein of CMV or an amino acid sequence having a sequence identity of at least 96% with SEQ ID NO:45. In another preferred embodiment, said CMV polypeptide comprises a coat protein of CMV or an amino acid sequence having a sequence identity of at least 97% with SEQ ID NO:45. In another preferred embodiment, said CMV polypeptide comprises a coat protein of CMV or an amino acid sequence having a sequence identity of at least 98% with SEQ ID NO:45. In another preferred embodiment, said CMV polypeptide comprises a coat protein of CMV or an amino acid sequence having a sequence identity of at least 99% with SEQ ID NO:45.

In a preferred embodiment, said CMV polypeptide consists of a coat protein of CMV or an amino acid sequence having a sequence identity of at least 75% with SEQ ID NO: 45. In another preferred embodiment, said CMV polypeptide consists of a coat protein of CMV or an amino acid sequence having a sequence identity of at least 80% with SEQ ID NO: 45. In another preferred embodiment, said CMV polypeptide consists of a coat protein of CMV or an amino acid sequence having a sequence identity of at least 85% with SEQ ID NO: 45. In another preferred embodiment, said CMV polypeptide consists of a coat protein of CMV or an amino acid sequence having a sequence identity of at least 90% with SEQ ID NO: 45. In another preferred embodiment, said CMV polypeptide consists of a coat protein of CMV or an amino acid sequence having a sequence identity of at least 92% with SEQ ID NO: 45. In another preferred embodiment, said CMV polypeptide consists of a coat protein of CMV or an amino acid sequence having a sequence identity of at least 93% with SEQ ID NO: 45. In another preferred embodiment, said CMV polypeptide consists of a coat protein of CMV or an amino acid sequence having a sequence identity of at least 95% with SEQ ID NO: 45. In another preferred embodiment, said CMV polypeptide consists of a coat protein of CMV or an amino acid sequence having a sequence identity of at least 96% with SEQ ID NO: 45. In another preferred embodiment, said CMV polypeptide consists of a coat protein of CMV or an amino acid sequence having a sequence identity of at least 97% with SEQ ID NO: 45. In another preferred embodiment, said CMV polypeptide consists of a coat protein of CMV or an amino acid sequence having a sequence identity of at least 98% with SEQ ID NO: 45. In another preferred embodiment, said CMV polypeptide consists of a coat protein of CMV or an amino acid sequence having a sequence identity of at least 99% with SEQ ID NO: 45.

In a preferred embodiment, said CMV polypeptide is a coat protein of CMV or an amino acid sequence having a sequence identity of at least 75%, preferably 85% with SEQ ID NO:45. In a preferred embodiment, said CMV polypeptide is a coat protein of CMV or an amino acid sequence having a sequence identity of at least 90%, preferably 95% with SEQ ID NO:45. In a preferred embodiment, said CMV polypeptide is a coat protein of CMV with SEQ ID NO:45. In a preferred embodiment, said coat protein of CMV comprises SEQ ID NO: 45. In a preferred embodiment, said coat protein of CMV consists of SEQ ID NO:45. In a preferred embodiment, said CMV polypeptide comprises a coat protein of CMV. In a preferred embodiment, said CMV polypeptide consists of a coat protein of CMV. In a preferred embodiment, said CMV polypeptide comprises a coat protein of CMV, wherein said coat protein of CMV comprises SEQ ID NO:45. In a preferred embodiment, said CMV polypeptide comprises a coat protein of CMV, wherein said coat protein of CMV consists of SEQ ID NO:45. In a preferred embodiment, said CMV polypeptide consists of a coat protein of CMV, wherein said coat protein of CMV consists of SEQ ID NO:45.

In a preferred embodiment, said CMV polypeptide comprises SEQ ID NO:46 or an amino acid sequence region, wherein said amino acid sequence region has a sequence identity of at least 75% with SEQ ID NO:46. In a preferred embodiment, said CMV polypeptide comprises SEQ ID NO:46 or an amino acid sequence region, wherein said amino acid sequence region has a sequence identity of at least 80% with SEQ ID NO:46. In a preferred embodiment, said CMV polypeptide comprises SEQ ID NO:46 or an amino acid sequence region, wherein said amino acid sequence region has a sequence identity of at least 85% with SEQ ID NO:46. In a preferred embodiment, said CMV polypeptide comprises SEQ ID NO:46 or an amino acid sequence region, wherein said amino acid sequence region has a sequence identity of at least 90% with SEQ ID NO:46. In a preferred embodiment, said CMV polypeptide comprises SEQ ID NO:46 or an amino acid sequence region, wherein said amino acid sequence region has a sequence identity of at least 95% with SEQ ID NO:46. In a preferred embodiment, said CMV polypeptide comprises SEQ ID NO:46 or an amino acid sequence region, wherein said amino acid sequence region has a sequence identity of at least 98% with SEQ ID NO:46. In a preferred embodiment, said CMV polypeptide comprises SEQ ID NO:46 or an amino acid sequence region, wherein said amino acid sequence region has a sequence identity of at least 99% with SEQ ID NO:46.

In a preferred embodiment, said CMV polypeptide comprises, or preferably consists of, (i) an amino acid sequence of a coat protein of CMV, wherein said amino acid sequence comprises, or preferably consists of, SEQ ID NO:45; or (ii) an amino acid sequence having a sequence identity of at least 90% of SEQ ID NO:45; and wherein said amino sequence as defined in (i) or (ii) comprises SEQ ID NO:46 or an amino acid sequence region, wherein said amino acid sequence region has a sequence identity of at least 90% with SEQ ID NO: 46. In a preferred embodiment, said CMV polypeptide comprises, or preferably consists of, (i) an amino acid sequence of a coat protein of CMV, wherein said amino acid sequence comprises, or preferably consists of, SEQ ID NO:45; or (ii) an amino acid sequence having a sequence identity of at least 95% of SEQ ID NO:45; and wherein said amino sequence as defined in (i) or (ii) comprises SEQ ID NO:46 or an amino acid sequence region, wherein said amino acid sequence region has a sequence identity of at least 95% with SEQ ID NO: 46. In a preferred embodiment, said CMV polypeptide comprises, or preferably consists of, (i) an amino acid sequence of a coat protein of CMV, wherein said amino acid sequence comprises, or preferably consists of, SEQ ID NO:45; or (ii) an amino acid sequence having a sequence identity of at least 90% of SEQ ID NO:45; and wherein said amino sequence as defined in (i) or (ii) comprises SEQ ID NO:46.

In a preferred embodiment, the number of amino acids of said N-terminal region replaced is equal to or lower than the number of amino acids of which said T helper cell epitope consists. In a preferred embodiment, said replaced N-terminal region of said CMV polypeptide consists of 5 to 15 consecutive amino acids. In a preferred embodiment, said replaced N-terminal region of said CMV polypeptide consists of 9 to 14 consecutive amino acids. In a preferred embodiment, said replaced N-terminal region of said CMV polypeptide consists of 11 to 13 consecutive amino acids. In a preferred embodiment, said N-terminal region of said CMV polypeptide corresponds to amino acids 2-12 of SEQ ID NO:45. In a preferred embodiment, said N-terminal region of said CMV polypeptide comprises amino acids 2-12 of SEQ ID NO:45. In a preferred embodiment, said N-terminal region of said CMV polypeptide consists of amino acids 2-12 of SEQ ID NO:45. In a preferred embodiment, said T helper cell epitope consists of at most 20 amino acids.

In a preferred embodiment of the present invention, the Th cell epitope is selected from TT 830-843 (SEQ ID NO:47), PADRE (SEQ ID NO:48), HA 307-319 (SEQ ID NO: 53), HBVnc 50-69 (SEQ ID NO:54), CS 378-398 (SEQ ID NO:55), MT 17-31 (SEQ ID NO: 56), and TT 947-967 (SEQ ID NO:57). In a preferred embodiment, said Th cell epitope is a Th cell epitope derived from tetanus toxin or is a PADRE sequence. In a preferred embodiment, said T helper cell epitope is derived from a human vaccine. In a preferred embodiment, said Th cell epitope is a Th cell epitope derived from tetanus toxin. In a preferred embodiment, said Th cell epitope is a PADRE sequence. In a preferred embodiment, said Th cell epitope comprises the amino acid sequence of SEQ ID NO:47 or SEQ ID NO:48. In a very preferred embodiment, said Th cell epitope consists of the amino acid sequence of SEQ ID NO:47 or SEQ ID NO:48. In a very preferred embodiment, said Th cell epitope comprises the amino acid sequence of SEQ ID NO:47. In a preferred embodiment, said Th cell epitope consists of the amino acid sequence of SEQ ID NO:47. In a very preferred embodiment, said Th cell epitope comprises the amino acid sequence of SEQ ID NO:48. In a very preferred embodiment, said Th cell epitope consists of the amino acid sequence of SEQ ID NO:48.

In a preferred embodiment, said CMV polypeptide comprises, or preferably consists of, an amino acid sequence of a coat protein of CMV, wherein said amino acid sequence comprises, or preferably consists of, SEQ ID NO:45 or an amino acid sequence having a sequence identity of at least 95% of SEQ ID NO:45; and wherein said amino sequence comprises SEQ ID NO:46, and wherein said T helper cell epitope replaces the N-terminal region of said CMV polypeptide, and wherein said replaced N-terminal region of said CMV polypeptide consists of 11 to 13 consecutive amino acids, preferably of 11 consecutive amino acids, and wherein further preferably said N-terminal region of said CMV polypeptide corresponds to amino acids 2-12 of SEQ ID NO:45. In a preferred embodiment, said chimeric CMV polypeptide comprises, preferably consists of, the amino acid sequence of SEQ ID NO:5, in which said polypeptide is inserted between any amino acid residue of said CMV polypeptide corresponding to any amino acid residue between position 75 and position 85 of SEQ ID NO:45. In another preferred embodiment, said chimeric CMV polypeptide comprises the amino acid sequence of SEQ ID NO:52, in which said polypeptide is inserted between any amino acid residue of said CMV polypeptide corresponding to any amino acid residue between position 75 and position 85 of SEQ ID NO: 45.

In a preferred embodiment, said stretch of consecutive negative amino acids has a length of 3 and less than 12 amino acids. In a preferred embodiment, said stretch of consecutive negative amino acids has a length of 3 to 10 amino acids. In a further preferred embodiment, said stretch of consecutive negative amino acids has a length of 3, 4, 5, 6, 7, 8, 9 or 10 amino acids. In a further preferred embodiment, said stretch of consecutive negative amino acids has a length of 3 to 9 amino acids. In a further preferred embodiment, said stretch of consecutive negative amino acids has a length of 3 to 8 amino acids. In a further preferred embodiment, said stretch of consecutive negative amino acids has a length of 4 to 9 amino acids. In a further preferred embodiment, said stretch of consecutive negative amino acids has a length of 4 to 8 amino acids. In a further preferred embodiment, said stretch of consecutive negative amino acids has a length of 4, 5, 6, 7 or 8. In a further preferred embodiment, said stretch of consecutive negative amino acids has a length of 4 or 8 amino acids. In a further preferred embodiment, said stretch of consecutive negative amino acids has a length of 3 amino acids. In a further preferred embodiment, said stretch of consecutive negative amino acids has a length of 4 amino acids. In a further preferred embodiment, said stretch of consecutive negative amino acids has a length of 5 amino acids. In a further preferred embodiment, said stretch of consecutive negative amino acids has a length of 6 amino acids. In a further preferred embodiment, said stretch of consecutive negative amino acids has a length of 7 amino acids. In a further preferred embodiment, said stretch of consecutive negative amino acids has a length of 8 amino acids. In a further preferred embodiment, said stretch of consecutive negative amino acids has a length of 9 amino acids.

In a further preferred embodiment, said stretch of consecutive negative amino acids are independently selected from aspartic acid or glutamic acid, wherein said aspartic acid or said glutamic acid is independently in each occasion selected from its L-configuration or its D-configuration. In a further preferred embodiment, said stretch of consecutive negative amino acids comprises at least one aspartic acid in the L-configuration or in the D-configuration. In a further preferred embodiment, said stretch of consecutive negative amino acids comprises at least one aspartic acid in the L-configuration. In a further preferred embodiment, said stretch of consecutive negative amino acids comprises at least one aspartic acid in the D-configuration. In a further preferred embodiment, said stretch of consecutive negative amino acids comprises at least one glutamic acid in the L-configuration or the D-configuration. In a further preferred embodiment, said stretch of consecutive negative amino acids comprises at least one glutamic acid in the L-configuration. In a further preferred embodiment, said stretch of consecutive negative amino acids comprises at least one glutamic acid in the D-configuration.

In a further preferred embodiment, said stretch of consecutive negative amino acids comprises at least one aspartic acid in the L-configuration and at least one glutamic acid in the L-configuration. In a further preferred embodiment, said stretch of consecutive negative amino acids consists of aspartic acid and glutamic acid, all in the L-configuration. In a further preferred embodiment, said stretch of consecutive negative amino acids consists of aspartic acid or glutamic acid, all in the L-configuration.

In a further preferred embodiment, said stretch of consecutive negative amino acids comprises at least one aspartic acid or at least one glutamic acid. In a further preferred embodiment, said stretch of consecutive negative amino acids comprises at least two aspartic acid or at least two glutamic acid. In a further preferred embodiment, said stretch of consecutive negative amino acids comprises at least three aspartic acid or at least three glutamic acid. In a further preferred embodiment, said stretch of consecutive negative amino acids comprises at least four aspartic acid or at least four glutamic acid. In a further preferred embodiment, said stretch of consecutive negative amino acids comprises at least four aspartic acid. In a further preferred embodiment, said stretch of consecutive negative amino acids comprises at least four glutamic acid. In a further preferred embodiment, said stretch of consecutive negative amino acids comprises at least five glutamic acid. In a further preferred embodiment, said stretch of consecutive negative amino acids comprises at least six glutamic acid. In a further preferred embodiment, said stretch of consecutive negative amino acids comprises at least seven glutamic acid. In a further preferred embodiment, said stretch of consecutive negative amino acids comprises at least eight glutamic acid. In a further preferred embodiment, said stretch of consecutive negative amino acids consist solely of aspartic acid. In a further very preferred embodiment, said stretch of consecutive negative amino acids consists solely of glutamic acids.

In a further preferred embodiment, said stretch of consecutive negative amino acids comprises at least one aspartic acid or at least one glutamic acid, wherein said at least one aspartic acid or said at least one glutamic acid are in the L-configuration. In a further preferred embodiment, said stretch of consecutive negative amino acids comprises at least two aspartic acid or at least two glutamic acid, wherein at least two aspartic acid or at least two glutamic acid are in the L-configuration. In a further preferred embodiment, said stretch of consecutive negative amino acids comprises at least three aspartic acid or at least three glutamic acid, wherein said at least three aspartic acid or said at least three glutamic acid are in the L-configuration. In a further preferred embodiment, said stretch of consecutive negative amino acids comprises at least four aspartic acid or at least four glutamic acid, wherein said at least four aspartic acid or said at least four glutamic acid are in the L-configuration. In a further preferred embodiment, said stretch of consecutive negative amino acids comprises at least four aspartic acid, wherein said at least four aspartic acid are in the L-configuration. In a further preferred embodiment, said stretch of consecutive negative amino acids comprises at least four glutamic acid, wherein said at least four glutamic acid are in the L-configuration. In a further preferred embodiment, said stretch of consecutive negative amino acids comprises at least five glutamic acid, wherein said at least five glutamic acid are in the L-configuration. In a further preferred embodiment, said stretch of consecutive negative amino acids comprises at least six glutamic acid, wherein said at least six glutamic acid are in the L-configuration. In a further preferred embodiment, said stretch of consecutive negative amino acids comprises at least seven glutamic acid, wherein said at least seven glutamic acid are in the L-configuration. In a further preferred embodiment, said stretch of consecutive negative amino acids comprises at least eight glutamic acid, wherein said at least eight glutamic acid are in the L-configuration. In a further preferred embodiment, said stretch of consecutive negative amino acids consist solely of aspartic acids, wherein said aspartic acids are in the L-configuration. In a further very preferred embodiment, said stretch of consecutive negative amino acids consists solely of glutamic acids, wherein said glutamic acids are in the L-configuration.

In a preferred embodiment, said stretch of consecutive negative amino acids has a length of 3 to 10 amino acids, wherein said stretch of consecutive negative amino acids consists solely of glutamic acids. In a further preferred embodiment, said stretch of consecutive negative amino acids has a length of 3, 4, 5, 6, 7, 8, 9 or 10 amino acids, wherein said stretch of consecutive negative amino acids consists solely of glutamic acids. In a further preferred embodiment, said stretch of consecutive negative amino acids has a length of 3 to 9 amino acids, wherein said stretch of consecutive negative amino acids consists solely of glutamic acids. In a further preferred embodiment, said stretch of consecutive negative amino acids has a length of 3 to 8 amino acids, wherein said stretch of consecutive negative amino acids consists solely of glutamic acids. In a further preferred embodiment, said stretch of consecutive negative amino acids has a length of 4 to 9 amino acids, wherein said stretch of consecutive negative amino acids consists solely of glutamic acids. In a further preferred embodiment, said stretch of consecutive negative amino acids has a length of 4 to 8 amino acids, wherein said stretch of consecutive negative amino acids consists solely of glutamic acids. In a further preferred embodiment, said stretch of consecutive negative amino acids has a length of 4, 5, 6, 7 or 8, wherein said stretch of consecutive negative amino acids consists solely of glutamic acids. In a further preferred embodiment, said stretch of consecutive negative amino acids has a length of 4 to 8 amino acids, wherein said stretch of consecutive negative amino acids consists solely of glutamic acids. In a further preferred embodiment, said stretch of consecutive negative amino acids has a length of 4, 5, 6, 7 or 8, wherein said stretch of consecutive negative amino acids consists solely of glutamic acids. In a further preferred embodiment, said stretch of consecutive negative amino acids has a length of 4 or 8 amino acids, wherein said stretch of consecutive negative amino acids consists solely of glutamic acids. In a further preferred embodiment, said stretch of consecutive negative amino acids has a length of 3 amino acids, wherein said stretch of consecutive negative amino acids consists solely of glutamic acids. In a further preferred embodiment, said stretch of consecutive negative amino acids has a length of 4 amino acids, wherein said stretch of consecutive negative amino acids consists solely of glutamic acids. In a further preferred embodiment, said stretch of consecutive negative amino acids has a length of 5 amino acids, wherein said stretch of consecutive negative amino acids consists solely of glutamic acids. In a further preferred embodiment, said stretch of consecutive negative amino acids has a length of 6 amino acids, wherein said stretch of consecutive negative amino acids consists solely of glutamic acids. In a further preferred embodiment, said stretch of consecutive negative amino acids has a length of 7 amino acids, wherein said stretch of consecutive negative amino acids consists solely of glutamic acids. In a further preferred embodiment, said stretch of consecutive negative amino acids has a length of 8 amino acids, wherein said stretch of consecutive negative amino acids consists solely of glutamic acids. In a further preferred embodiment, said stretch of consecutive negative amino acids has a length of 9 amino acids, wherein said stretch of consecutive negative amino acids consists solely of glutamic acids.

In a preferred embodiment, said stretch of consecutive negative amino acids has a length of 3 to 10 amino acids, wherein said stretch of consecutive negative amino acids consists solely of glutamic acids, wherein said glutamic acids are in the L-configuration. In a further preferred embodiment, said stretch of consecutive negative amino acids has a length of 3, 4, 5, 6, 7, 8, 9 or 10 amino acids, wherein said stretch of consecutive negative amino acids consists solely of glutamic acids, wherein said glutamic acids are in the L-configuration. In a further preferred embodiment, said stretch of consecutive negative amino acids has a length of 3 to 9 amino acids, wherein said stretch of consecutive negative amino acids consists solely of glutamic acids, wherein said glutamic acids are in the L-configuration. In a further preferred embodiment, said stretch of consecutive negative amino acids has a length of 3 to 8 amino acids, wherein said stretch of consecutive negative amino acids consists solely of glutamic acids, wherein said glutamic acids are in the L-configuration. In a further preferred embodiment, said stretch of consecutive negative amino acids has a length of 4 to 9 amino acids, wherein said stretch of consecutive negative amino acids consists solely of glutamic acids, wherein said glutamic acids are in the L-configuration. In a further preferred embodiment, said stretch of consecutive negative amino acids has a length of 4 to 8 amino acids, wherein said stretch of consecutive negative amino acids consists solely of glutamic acids, wherein said glutamic acids are in the L-configuration. In a further preferred embodiment, said stretch of consecutive negative amino acids has a length of 4, 5, 6, 7 or 8, wherein said stretch of consecutive negative amino acids consists solely of glutamic acids, wherein said glutamic acids are in the L-configuration. In a further preferred embodiment, said stretch of consecutive negative amino acids has a length of 4 to 8 amino acids, wherein said stretch of consecutive negative amino acids consists solely of glutamic acids, wherein said glutamic acids are in the L-configuration. In a further preferred embodiment, said stretch of consecutive negative amino acids has a length of 4, 5, 6, 7 or 8, wherein said stretch of consecutive negative amino acids consists solely of glutamic acids, wherein said glutamic acids are in the L-configuration. In a further preferred embodiment, said stretch of consecutive negative amino acids has a length of 4 or 8 amino acids, wherein said stretch of consecutive negative amino acids consists solely of glutamic acids, wherein said glutamic acids are in the L-configuration. In a further preferred embodiment, said stretch of consecutive negative amino acids has a length of 3 amino acids, wherein said stretch of consecutive negative amino acids consists solely of glutamic acids, wherein said glutamic acids are in the L-configuration. In a further preferred embodiment, said stretch of consecutive negative amino acids has a length of 4 amino acids, wherein said stretch of consecutive negative amino acids consists solely of glutamic acids, wherein said glutamic acids are in the L-configuration. In a further preferred embodiment, said stretch of consecutive negative amino acids has a length of 5 amino acids, wherein said stretch of consecutive negative amino acids consists solely of glutamic acids, wherein said glutamic acids are in the L-configuration. In a further preferred embodiment, said stretch of consecutive negative amino acids has a length of 6 amino acids, wherein said stretch of consecutive negative amino acids consists solely of glutamic acids, wherein said glutamic acids are in the L-configuration. In a further preferred embodiment, said stretch of consecutive negative amino acids has a length of 7 amino acids, wherein said stretch of consecutive negative amino acids consists solely of glutamic acids, wherein said glutamic acids are in the L-configuration. In a further preferred embodiment, said stretch of consecutive negative amino acids has a length of 8 amino acids, wherein said stretch of consecutive negative amino acids consists solely of glutamic acids, wherein said glutamic acids are in the L-configuration. In a further preferred embodiment, said stretch of consecutive negative amino acids has a length of 9 amino acids, wherein said stretch of consecutive negative amino acids consists solely of glutamic acids, wherein said glutamic acids are in the L-configuration.

In a further very preferred embodiment, said stretch of consecutive negative amino acids comprises SEQ ID NO:1 or SEQ ID NO:2. In a further very preferred embodiment, said stretch of consecutive negative amino acids consists of SEQ ID NO:1 or SEQ ID NO:2. In a further very preferred embodiment, said stretch of consecutive negative amino acids comprises SEQ ID NO:1. In a further very preferred embodiment, said stretch of consecutive negative amino acids consists of SEQ ID NO:1. In a further very preferred embodiment, said stretch of consecutive negative amino acids comprises SEQ ID NO:2. In a further very preferred embodiment, said stretch of consecutive negative amino acids consists of SEQ ID NO:2.

In a preferred embodiment, said polypeptide comprising said stretch of consecutive negative amino acids further comprises a first amino acid linker, wherein said first amino acid linker is positioned at the N- or at the C-terminus of said stretch of consecutive negative amino acids. In a preferred embodiment, said polypeptide further comprises a first amino acid linker, wherein said first amino acid linker is positioned at the N-terminus of said stretch of consecutive negative amino acids. In a preferred embodiment, said polypeptide comprising said stretch of consecutive negative amino acids further comprises a first amino acid linker, wherein said first amino acid linker is positioned at the C-terminus of said stretch of consecutive negative amino acids. In a preferred embodiment, said polypeptide comprising said stretch of consecutive negative amino acids further comprises a second amino acid linker. In a preferred embodiment, said polypeptide comprising said stretch of consecutive negative amino acids further comprises a first amino acid linker and a second amino acid linker, wherein said first amino acid linker is positioned at the N-terminus of said stretch of consecutive negative amino acids, and said second amino acid linker is positioned at the C-terminus of said stretch of consecutive negative amino acids.

In a preferred embodiment, said first amino acid linker has a length of at most 30 amino acids. In a preferred embodiment, said first amino acid linker has a length of at most 20, 19, 18, 17 or 16 amino acids. In a preferred embodiment, said first amino acid linker has a length of at most 15 amino acids. In a preferred embodiment, said first amino acid linker has a length of at most 14 amino acids. In a preferred embodiment, said first amino acid linker has a length of at most 13 amino acids. In a preferred embodiment, said first amino acid linker has a length of at most 12 amino acids. In a preferred embodiment, said first amino acid linker has a length of at most 11 amino acids. In a preferred embodiment, said first amino acid linker has a length of at most 10 amino acids. In a preferred embodiment, said first amino acid linker has a length of at most 9 amino acids. In a preferred embodiment, said first amino acid linker has a length of at most 8 amino acids. In a preferred embodiment, said first amino acid linker has a length of at most 7 amino acids. In a preferred embodiment, said first amino acid linker has a length of at most 6 amino acids. In a preferred embodiment, said first amino acid linker has a length of at most 5 amino acids. In a preferred embodiment, said first amino acid linker has a length of at most 4 amino acids. In a preferred embodiment, said first amino acid linker has a length of at most 3 amino acids. In a preferred embodiment, said first amino acid linker has a length of at most 2 amino acids. In a preferred embodiment, said first amino acid linker consists of one amino acid. In a preferred embodiment, said second amino acid linker has a length of at most 30 amino acids. In a preferred embodiment, said second amino acid linker has a length of at most 20, 19, 18, 17 or 16 amino acids. In a preferred embodiment, said second amino acid linker has a length of at most 15 amino acids. In a preferred embodiment, said second amino acid linker has a length of at most 14 amino acids. In a preferred embodiment, said second amino acid linker has a length of at most 13 amino acids. In a preferred embodiment, said second amino acid linker has a length of at most 12 amino acids. In a preferred embodiment, said second amino acid linker has a length of at most 11 amino acids. In a preferred embodiment, said second amino acid linker has a length of at most 10 amino acids. In a preferred embodiment, said second amino acid linker has a length of at most 9 amino acids. In a preferred embodiment, said second amino acid linker has a length of at most 8 amino acids. In a preferred embodiment, said second amino acid linker has a length of at most 7 amino acids. In a preferred embodiment, said second amino acid linker has a length of at most 6 amino acids. In a preferred embodiment, said second amino acid linker has a length of at most 5 amino acids. In a preferred embodiment, said second amino acid linker has a length of at most 4 amino acids. In a preferred embodiment, said second amino acid linker has a length of at most 3 amino acids. In a preferred embodiment, said second amino acid linker has a length of at most 2 amino acids. In a preferred embodiment, said second amino acid linker consists of one amino acid.

In a preferred embodiment, said polypeptide comprising said stretch of consecutive negative amino acids further comprises a first amino acid linker, wherein said first amino acid linker is positioned at the N- or at the C-terminus of said stretch of consecutive negative amino acids, and wherein said first amino acid linker is selected from the group consisting of: (a.) a polyglycine linker (Gly) n of a length of n=2-10; (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and (c.) an amino acid linker (GS*-linker) comprising at least one Gly, at least one Ser, and at least one amino acid selected from Thr, Ala, Lys, and Cys.

In a preferred embodiment, said polypeptide comprising said stretch of consecutive negative amino acids further comprises a second amino acid linker, wherein said second amino acid linker is positioned at the N- or at the C-terminus of said stretch of consecutive negative amino acids, and wherein said second amino acid linker is selected from the group consisting of: (a.) a polyglycine linker (Gly) n of a length of n=2-10; (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and (c.) an amino acid linker (GS*-linker) comprising at least one Gly, at least one Ser, and at least one amino acid selected from Thr, Ala, Lys, and Cys. In a preferred embodiment, said polypeptide comprising said stretch of consecutive negative amino acids further comprises a first amino acid linker and a second amino acid linker, wherein said first amino acid linker is positioned at the N-terminus of said stretch of consecutive negative amino acids, and said second amino acid linker is positioned at the C-terminus of said stretch of consecutive negative amino acids, and wherein said first and said second amino acid linker is independently selected from the group consisting of (a.) a polyglycine linker (G-linker) having an amino acid sequence (Gly)ₙ of a length of n=2-10; (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and (c.) an amino acid linker (GS*-linker) comprising at least one Gly, at least one Ser, and at least one amino acid selected from Thr, Ala, Lys, and Cys.

In a preferred embodiment, said polypeptide comprising said stretch of consecutive negative amino acids further comprises a first amino acid linker and a second amino acid linker, wherein said first amino acid linker is positioned at the N-terminus of said stretch of consecutive negative amino acids, and said second amino acid linker is positioned at the C-terminus of said stretch of consecutive negative amino acids, and wherein said first and said second amino acid linker are independently selected from a glycine-serine linker (GS-linker) comprising at least one glycine and at least one serine, wherein said GS linker has an amino acid sequence of (GS)ᵣ(GₛS)ₜ(GS)ᵤ with r=0 or 1, s=1-5, t=1-5 and u=0 or 1; and an amino acid linker (GS*-linker) comprising at least one Gly, at least one Ser, and at least one amino acid selected from Thr, Ala, Lys, and Cys.

In a preferred embodiment, said first amino acid linker is a polyglycine linker (Gly) n of a length of n=2-10. In a preferred embodiment, said first amino acid linker is a glycine-serine linker (GS-linker) comprising at least one glycine and at least one serine. In a preferred embodiment, said first amino acid linker is a glycine-serine linker (GS-linker) comprising at least one glycine and at least one serine, and wherein said first amino acid linker has a Gly-Ser at its N-terminus. In a further preferred embodiment, said GS linker has an amino acid sequence of (GS)ᵣ(GₛS)ₜ(GS)ᵤ with r=0 or 1, s=1-5, t=1-5 and u=0 or 1. In a further preferred embodiment, said first amino acid linker is a glycine-serine linker (GS-linker), said GS linker has an amino acid sequence of (GS)ᵣ(GₛS)ₜ(GS)ᵤ with r=0 or 1, s=3 or 4, t=1, 2 or 3, and u=0 or 1. In a further preferred embodiment, said GS-linker has a length of at most 15, 14, 13, 12, 11, preferably 10, 9, 8, 7, and further preferably a length of at most 6 amino acids. In a further preferred embodiment, said first amino acid linker is a glycine-serine linker (GS-linker), and said GS linker has an amino acid sequence of SEQ ID NO:8. In a further preferred embodiment, said first amino acid linker has an amino acid sequence of SEQ ID NO:8. In a preferred embodiment, said first amino acid linker is an amino acid linker (GS*-linker) comprising at least one Gly, at least one Ser, and at least one amino acid selected from Thr, Ala, Lys, and Cys.

In a preferred embodiment, said second amino acid linker is a polyglycine linker (Gly) n of a length of n=2-10. In a preferred embodiment, said second amino acid linker is a glycine-serine linker (GS-linker) consisting of at least one glycine and at least one serine. In a preferred embodiment, said second amino acid linker is a glycine-serine linker (GS-linker) comprising at least one glycine and at least one serine, and wherein said second amino acid linker has a Gly-Ser at its N-terminus. In a further preferred embodiment, said second amino acid linker is a glycine-serine linker (GS-linker), said GS linker has an amino acid sequence of (GS)ᵣ(GₛS)ₜ(GS)ᵤ with r=0 or 1, s=3 or 4, t=1, 2 or 3, u=0 or 1. In a further preferred embodiment, said GS-linker has a length of at most 15, 14, 13, 12, 11, preferably 10, 9, 8, 7, and further preferably a length of at most 6 amino acids. In a further preferred embodiment, said second amino acid linker is a glycine-serine linker (GS-linker), and said GS linker has the amino acid sequence of SEQ ID NO:9.

In a preferred embodiment, said second amino acid linker is an amino acid linker (GS*-linker) comprising at least one Gly, at least one Ser, and at least one amino acid selected from Thr, Ala, Lys, and Cys. In a preferred embodiment, said second amino acid linker is an amino acid linker (GS*-linker) comprising at least one Gly, at least one Ser, and at least Cys. In a preferred embodiment, said second amino acid linker is an amino acid linker comprising at least one Gly, at least one Ser, and at least Cys (GS*-linker), and said second amino acid linker has a Gly-Ser at its N-terminus. In a further preferred embodiment, said second amino acid linker (GS*-linker) has a length of at most 15, 14, 13, 12, 11, preferably 10, 9, and further preferably a length of at most 7 or 6 amino acids. In a further preferred embodiment, said second amino acid linker is amino acid linker (GS*-linker), and said GS*-linker has the amino acid sequence of SEQ ID NO:4.

In a preferred embodiment, said first and said second amino acid linker are independently a polyglycine linker (Gly)ₙ of a length of n=2-10. In a preferred embodiment, said first and said second amino acid linker are independently a glycine-serine linker (GS-linker) comprising at least one glycine and at least one serine. In a preferred embodiment, said first and said second amino acid linker are independently an amino acid linker (GS*-linker) comprising at least one Gly, at least one Ser, and at least one amino acid selected from Thr, Ala, Lys, and Cys, and wherein said second amino acid linker has a Gly-Ser at its N-terminus. In a further preferred embodiment, said GS linker has an amino acid sequence of (GS)ᵣ(GₛS)ₜ(GS)ᵤ with r=0 or 1, s=1-5, t=1-5 and u=0 or 1. In a further preferred embodiment, said first and said second amino acid linker is independently a glycine-serine linker (GS-linker), said GS linker has an amino acid sequence of (GS)ᵣ(GₛS)ₜ(GS)ᵤ with r=0 or 1, s=2, 3 or 4, t=1, 2 or 3, u=0 or 1.

In a further preferred embodiment, said first amino acid linker and/or said second amino linker comprises, preferably consists of, of an amino acid sequence selected from SEQ ID NO:4, SEQ ID NO:8 and SEQ ID NO:9. In a further very preferred embodiment, said first amino acid linker comprises, preferably consists of, SEQ ID NO:8. In a further very preferred embodiment, said second amino acid linker comprises, preferably consists of, SEQ ID NO:4 or SEQ ID NO:9. In a further very preferred embodiment, said second amino acid linker comprises, preferably consists of, SEQ ID NO:4. In a further very preferred embodiment, said second amino acid linker comprises, preferably consists of, SEQ ID NO:9. In a further very preferred embodiment, said first amino acid linker comprises, preferably consists of, SEQ ID NO:8 and said second amino acid linker comprises, preferably consists of, SEQ ID NO:4 or SEQ ID NO:9. In a further very preferred embodiment, said first amino acid linker comprises, preferably consists of, SEQ ID NO:8 and said second amino acid linker comprises, preferably consists of, SEQ ID NO:4. In a further very preferred embodiment, said first amino acid linker comprises, preferably consists of, SEQ ID NO:8 and said second amino acid linker comprises, preferably consists of, or SEQ ID NO:9.

In a preferred embodiment, said polypeptide comprising, preferably consisting of, a stretch of consecutive negative amino acids has a length of at most 30 amino acids. In a preferred embodiment, said polypeptide has a length of at most 25, 24, 23, 22, or 21 amino acids. In a preferred embodiment, said polypeptide has a length of at most 20 amino acids. In a preferred embodiment, said polypeptide has a length of at most 19 amino acids. In a preferred embodiment, said polypeptide has a length of at most 18 amino acids. In a preferred embodiment, said polypeptide has a length of at most 17 amino acids. In a preferred embodiment, said polypeptide has a length of at most 16 amino acids. In a preferred embodiment, said polypeptide has a length of at most 15 amino acids. In a preferred embodiment, said polypeptide has a length of at most 14 amino acids. In a preferred embodiment, said polypeptide has a length of at most 13 amino acids In a preferred embodiment, said polypeptide has a length of at most 12 amino acids. In a preferred embodiment, said polypeptide has a length of at most 11 amino acids. In a preferred embodiment, said polypeptide has a length of at most 10 amino acids. In a preferred embodiment, said polypeptide has a length of at most 9 amino acids. In a preferred embodiment, said polypeptide has a length of at most 8 amino acids. In a preferred embodiment, said polypeptide has a length of at most 7 amino acids. In a preferred embodiment, said polypeptide has a length of at most 6 amino acids. In a preferred embodiment, said polypeptide has a length of at most 5 amino acids. In a preferred embodiment, said polypeptide has a length of at most 4 amino acids. In a further preferred embodiment, said polypeptide consists of said stretch of consecutive negative amino acids.

In a further very preferred embodiment, said polypeptide comprises SEQ ID NO: 49, SEQ ID NO:50 or SEQ ID NO:51. In a further very preferred embodiment, said polypeptide consists of SEQ ID NO:49, SEQ ID NO:50 or SEQ ID NO:51. In a further very preferred embodiment, said polypeptide comprises SEQ ID NO:49. In a further very preferred embodiment, said polypeptide comprises SEQ ID NO:50. In a further very preferred embodiment, said polypeptide comprises SEQ ID NO:51. In a further very preferred embodiment, said polypeptide consists of SEQ ID NO:49. In a further very preferred embodiment, said polypeptide consists of SEQ ID NO:50. In a further very preferred embodiment, said polypeptide consists of SEQ ID NO:51.

In a further preferred embodiment, said polypeptide comprising, preferably consisting of, a stretch of consecutive negative amino acids is inserted between any amino acid residue of said CMV polypeptide corresponding to any amino acid residue between position 75 and position 85 of SEQ ID NO:45. In a further preferred embodiment, said polypeptide comprising, preferably consisting of, a stretch of consecutive negative amino acids is inserted between amino acid residues of said CMV polypeptide corresponding to amino acid residues of position 75 and position 76 of SEQ ID NO:45. In a further preferred embodiment, said polypeptide comprising, preferably consisting of, a stretch of consecutive negative amino acids is inserted between amino acid residues of said CMV polypeptide corresponding to amino acid residues of position 76 and position 77 of SEQ ID NO:45. In a further preferred embodiment, said polypeptide comprising, preferably consisting of, a stretch of consecutive negative amino acids is inserted between amino acid residues of said CMV polypeptide corresponding to amino acid residues of position 77 and position 78 of SEQ ID NO:45. In a further preferred embodiment, said polypeptide comprising, preferably consisting of, a stretch of consecutive negative amino acids is inserted between amino acid residues of said CMV polypeptide corresponding to amino acid residues of position 78 and position 79 of SEQ ID NO:45. In a further preferred embodiment, said polypeptide comprising, preferably consisting of, a stretch of consecutive negative amino acids is inserted between amino acid residues of said CMV polypeptide corresponding to amino acid residues of position 79 and position 80 of SEQ ID NO:45. In a further preferred embodiment, said polypeptide comprising, preferably consisting of, a stretch of consecutive negative amino acids is inserted between amino acid residues of said CMV polypeptide corresponding to amino acid residues of position 80 and position 81 of SEQ ID NO:45. In a further preferred embodiment, said polypeptide comprising, preferably consisting of, a stretch of consecutive negative amino acids is inserted between amino acid residues of said CMV polypeptide corresponding to amino acid residues of position 75 and position 81 of SEQ ID NO:45. In a further preferred embodiment, said polypeptide comprising, preferably consisting of, a stretch of consecutive negative amino acids is inserted between amino acid residues of said CMV polypeptide corresponding to amino acid residues of position 82 and position 83 of SEQ ID NO:45. In a further preferred embodiment, said polypeptide comprising, preferably consisting of, a stretch of consecutive negative amino acids is inserted between amino acid residues of said CMV polypeptide corresponding to amino acid residues of position 83 and position 84 of SEQ ID NO:45. In a further very preferred embodiment, said polypeptide comprising, preferably consisting of, a stretch of consecutive negative amino acids is inserted between amino acid residues of said CMV polypeptide corresponding to amino acid residues of position 84 and position 85 of SEQ ID NO:45.

In a very preferred embodiment, said CMV polypeptide comprises the amino acid sequence of SEQ ID NO:5, SEQ ID NO:45 or SEQ ID NO:52, wherein said polypeptide comprising said stretch of consecutive negative amino acids is inserted between amino acid residues of position 88 (Ser) and position 89 (Thr) of said SEQ ID NO:5, between amino acid residues of position 84 (Ser) and position 85 (Thr) of SEQ ID NO:45, or between amino acid residues of position 86 (Ser) and position 87 (Thr) of SEQ ID NO:52. In a very preferred embodiment, said CMV polypeptide consists of the amino acid sequence of SEQ ID NO:5, SEQ ID NO:45 or SEQ ID NO:52, wherein said polypeptide comprising said stretch of consecutive negative amino acids is inserted between amino acid residues of position 88 and position 89 of said SEQ ID NO:5, between amino acid residues of position 84 and position 85 of SEQ ID NO:45, or between amino acid residues of position 86 and position 87 of SEQ ID NO: 52. In a very preferred embodiment, said CMV polypeptide comprises the amino acid sequence of SEQ ID NO:5, wherein said polypeptide is inserted between amino acid residues of position 88 and position 89 of SEQ ID NO:5. In a very preferred embodiment, said CMV polypeptide comprises the amino acid sequence of SEQ ID NO:45, wherein said polypeptide is inserted between amino acid residues of position 84 and position 85 of SEQ ID NO: 45. In a very preferred embodiment, said CMV polypeptide comprises the amino acid sequence of SEQ ID NO:52, wherein said polypeptide is inserted between amino acid residues of position 86 and position 87 of SEQ ID NO:52. In a very preferred embodiment, said CMV polypeptide consists of the amino acid sequence of SEQ ID NO:5, wherein said polypeptide is inserted between amino acid residues of position 88 and position 89 of SEQ ID NO: 5. In a very preferred embodiment, said CMV polypeptide consists of the amino acid sequence of SEQ ID NO:45, wherein said polypeptide is inserted between amino acid residues of position 84 and position 85 of SEQ ID NO:45. In a very preferred embodiment, said CMV polypeptide consists of the amino acid sequence of SEQ ID NO:52, wherein said polypeptide is inserted between amino acid residues of position 86 and position 87 of SEQ ID NO: 52.

In a very preferred embodiment, said chimeric CMV polypeptide comprises the amino acid sequence of SEQ ID NO:5, SEQ ID NO:45 or SEQ ID NO:52, wherein said polypeptide comprising said stretch of consecutive negative amino acids is inserted between amino acid residues of position 88 (Ser) and position 89 (Thr) of said SEQ ID NO:5, between amino acid residues of position 84 (Ser) and position 85 (Thr) of SEQ ID NO:45, or between amino acid residues of position 86 (Ser) and position 87 (Thr) of SEQ ID NO:52. In a very preferred embodiment, said chimeric CMV polypeptide consists of the amino acid sequence of SEQ ID NO:5, SEQ ID NO:45 or SEQ ID NO:52, wherein said polypeptide comprising said stretch of consecutive negative amino acids is inserted between amino acid residues of position 88 and position 89 of said SEQ ID NO:5, between amino acid residues of position 84 and position 85 of SEQ ID NO:45, or between amino acid residues of position 86 and position 87 of SEQ ID NO:52. In a very preferred embodiment, said chimeric CMV polypeptide comprises the amino acid sequence of SEQ ID NO:5, wherein said polypeptide is inserted between amino acid residues of position 88 and position 89 of SEQ ID NO:5. In a very preferred embodiment, said chimeric CMV polypeptide comprises the amino acid sequence of SEQ ID NO:45, wherein said polypeptide is inserted between amino acid residues of position 84 and position 85 of SEQ ID NO:45. In a very preferred embodiment, said chimeric CMV polypeptide comprises the amino acid sequence of SEQ ID NO:52, wherein said polypeptide is inserted between amino acid residues of position 86 and position 87 of SEQ ID NO:52. In a very preferred embodiment, said chimeric CMV polypeptide consists of the amino acid sequence of SEQ ID NO:5, wherein said polypeptide is inserted between amino acid residues of position 88 and position 89 of SEQ ID NO:5. In a very preferred embodiment, said chimeric CMV polypeptide consists of the amino acid sequence of SEQ ID NO:45, wherein said polypeptide is inserted between amino acid residues of position 84 and position 85 of SEQ ID NO:45. In a very preferred embodiment, said chimeric CMV polypeptide consists of the amino acid sequence of SEQ ID NO:52, wherein said polypeptide is inserted between amino acid residues of position 86 and position 87 of SEQ ID NO: 52.

In a very preferred embodiment, said CMV polypeptide comprises the amino acid sequence of SEQ ID NO:5 and said polypeptide comprising said stretch of consecutive negative amino acids is inserted between amino acid residues 88 (Ser) and amino acid residue 89 (Thr) of SEQ ID NO:5, and said polypeptide comprising said stretch of consecutive negative amino acids further comprises a first and a second amino acid linker, wherein said first and said second amino acid linker is independently a glycine-serine linker (GS-linker) comprising at least one glycine and at least one serine or an amino acid linker (GS*-linker) comprising at least one Gly, at least one Ser, and at least one amino acid selected from Thr, Ala, Lys, and Cys, wherein said first and/or said second amino acid linker has a Gly-Ser sequence at its N-terminus.

In a very preferred embodiment, said chimeric CMV polypeptide comprises the amino acid sequence of SEQ ID NO:5 and said polypeptide comprising said stretch of consecutive negative amino acids is inserted between amino acid residues 88 (Ser) and amino acid residue 89 (Thr) of SEQ ID NO:5, and said polypeptide comprising said stretch of consecutive negative amino acids further comprises a first and a second amino acid linker, wherein said first and said second amino acid linker is independently a glycine-serine linker (GS-linker) comprising at least one glycine and at least one serine or an amino acid linker (GS*-linker) comprising at least one Gly, at least one Ser, and at least one amino acid selected from Thr, Ala, Lys, and Cys, wherein said first and/or said second amino acid linker has a Gly-Ser sequence at its N-terminus.

In a very preferred embodiment, said chimeric CMV polypeptide comprises the amino acid sequence of SEQ ID NO:10, SEQ ID NO:11 or SEQ ID NO:12. In a very preferred embodiment, said chimeric CMV polypeptide comprises the amino acid sequence of SEQ ID NO:10. In a very preferred embodiment, said chimeric CMV polypeptide comprises the amino acid sequence of SEQ ID NO: 11. In a very preferred embodiment, said chimeric CMV polypeptide comprises the amino acid sequence of SEQ ID NO: 12. In a very preferred embodiment, said chimeric CMV polypeptide consists of the amino acid sequence of SEQ ID NO:10, SEQ ID NO:11 or SEQ ID NO:12. In a very preferred embodiment, said chimeric CMV polypeptide consists of the amino acid sequence of SEQ ID NO: 10. In a very preferred embodiment, said chimeric CMV polypeptide consists of the amino acid sequence of SEQ ID NO: 11. In a very preferred embodiment, said chimeric CMV polypeptide consists of the amino acid sequence of SEQ ID NO:12.

Thus, in another aspect, the present invention provides a composition, preferably a veterinary composition, comprising
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of, the amino acid sequence of SEQ ID NO: 10, SEQ ID NO: 11 or SEQ ID NO:12;
(b) at least one antigen, wherein said antigen comprises at least one second attachment site, and wherein said antigen is an interleukin 1β D145X mutein (IL-1β-D145X mutein) antigen, preferably an interleukin 1β D145K mutein (IL-1β-D145K mutein) antigen; and
   wherein (a) and (b) are linked through said at least one first and said at least one second attachment site, preferably via at least one covalent non-peptide bond.

In embodiment, said chimeric CMV polypeptide comprises the amino acid sequence of SEQ ID NO:10. In a very preferred embodiment, said chimeric CMV polypeptide comprises the amino acid sequence of SEQ ID NO:11. In a very preferred embodiment, said chimeric CMV polypeptide comprises the amino acid sequence of SEQ ID NO: 12. In a very preferred embodiment, said chimeric CMV polypeptide consists of the amino acid sequence of SEQ ID NO:10, SEQ ID NO:11 or SEQ ID NO:12. In a very preferred embodiment, said chimeric CMV polypeptide consists of the amino acid sequence of SEQ ID NO:10. In a very preferred embodiment, said chimeric CMV polypeptide consists of the amino acid sequence of SEQ ID NO:11. In a very preferred embodiment, said chimeric CMV polypeptide consists of the amino acid sequence of SEQ ID NO: 12. In a very preferred embodiment, said modified VLP of CMV comprises 180 identical chimeric CMV polypeptides, wherein said chimeric CMV polypeptide comprises, preferably consists of, the amino acid sequence of SEQ ID NO:10, SEQ ID NO:11 or SEQ ID NO:12. In a very preferred embodiment, said modified VLP of CMV comprises 180 identical chimeric CMV polypeptides, wherein said chimeric CMV polypeptide comprises, preferably consists of, the amino acid sequence of SEQ ID NO: 10. In a very preferred embodiment, said modified VLP of CMV comprises 180 identical chimeric CMV polypeptides, wherein said chimeric CMV polypeptide comprises, preferably consists of, the amino acid sequence of SEQ ID NO:11. In a very preferred embodiment, said modified VLP of CMV comprises 180 identical chimeric CMV polypeptides, wherein said chimeric CMV polypeptide comprises, preferably consists of, the amino acid sequence of SEQ ID NO:12.

The modified CMV VLPs of the invention may be expressed in prokaryotic or eukaryotic expression systems. Preferred systems are E. coli, yeast, insect cells as well as mammalian cell lines. Very preferred said modified VLP of CMV is obtained by expression of said chimeric CMV polypeptide in E. coli, and wherein preferably said expression is effected at temperatures of between 10° C. to 25° C., preferably at a temperature of 20° C. As indicated above, recombinantly produced polypeptides may comprise an N-terminal methionine residue. In one embodiment said chimeric CMV polypeptide therefore comprises an N-terminal methionine residue. However, typically and preferably said N-terminal methionine residue is cleaved off said chimeric CMV polypeptide.

In a further preferred embodiment said modified VLP of CMV further comprises at least one immunostimulatory substance. In a very preferred embodiment, said immunostimulatory substance is packaged into the modified VLPs of the invention. In another preferred embodiment, the immunostimulatory substance is mixed with the modified VLPs of the invention. Immunostimulatory substances useful for the invention are generally known in the art and are disclosed, inter alia, in WO2003/024481.

In another embodiment of the present invention, said immunostimulatory substance consists of DNA or RNA of non-eukaryotic origin. In a further preferred embodiment said immunostimulatory substance is selected from the group consisting of: (a) immunostimulatory nucleic acid; (b) peptidoglycan; (c) lipopolysaccharide; (d) lipoteichonic acid; (e) imidazoquinoline compound; (f) flagelline; (g) lipoprotein; and (h) any mixtures of at least one substance of (a) to (g). In a further preferred embodiment said immunostimulatory substance is an immunostimulatory nucleic acid, wherein said immunostimulatory nucleic acid is selected from the group consisting of: (a) ribonucleic acids; (b) deoxyribonucleic acids; (c) chimeric nucleic acids; and (d) any mixture of (a), (b) and/or (c). In a further preferred embodiment said immunostimulatory nucleic acid is a ribonucleic acid, and wherein said ribonucleic acid is bacteria derived RNA. In a further preferred embodiment said immunostimulatory nucleic acid is poly (IC) or a derivative thereof. In a further preferred embodiment said immunostimulatory nucleic acid is a deoxyribonucleic acid, wherein said deoxyribonucleic acid is an unmethylated CpG-containing oligonucleotide.

In a very preferred embodiment said immunostimulatory substance is an unmethylated CpG-containing oligonucleotide. In a further preferred embodiment said unmethylated CpG-containing oligonucleotide is an A-type CpG. In a further preferred embodiment said A-type CpG comprises a palindromic sequence. In a further preferred embodiment said palindromic sequence is flanked at its 5′-terminus and at its 3 ‘-terminus by guanosine entities. In a further preferred embodiment said palindromic sequence is flanked at its 5’-terminus by at least 3 and at most 15 guanosine entities, and wherein said palindromic sequence is flanked at its 3′-terminus by at least 3 and at most 15 guanosine entities.

In another preferred embodiment, said immunostimulatory substance is an unmethylated CpG-containing oligonucleotide, and wherein preferably said unmethylated CpG-containing oligonucleotide comprises a palindromic sequence, and wherein further preferably the CpG motif of said unmethylated CpG-containing oligonucleotide is part of a palindromic sequence, and wherein again further preferably said palindromic sequence is SEQ ID NO:58. In a further preferred embodiment, said immunostimulatory nucleic acid is an unmethylated CpG containing oligonucleotide consisting of SEQ ID NO:59, wherein said unmethylated CpG-containing oligonucleotide consists exclusively of phosphodiester bound nucleotides.

In a further aspect, the present invention provides a composition comprising (a) modified VLP of CMV as defined herein, wherein said modified VLP of CMV comprises at least one first attachment site; and (b) at least one interleukin 1β D145X mutein (IL-1β-D145X mutein) antigen, preferably at least one interleukin 1β D145K mutein (IL-1β-D145K mutein) antigen, wherein said antigen comprises at least one second attachment site; wherein (a) and (b) are linked through said at least one first and said at least one second attachment site, typically and preferably via at least one covalent non-peptide bond. Methods for linking said modified VLP and said antigens via said first and said second attachment site are described, for example, in WO2002/056905, WO2004/084940 and WO2016/062720.

Thus, in a further aspect, the present invention provides a composition comprising (a) modified VLP of CMV, wherein said modified VLP of CMV comprises at least one first attachment site; and (b) at least one IL-1β-D145X mutein antigen, preferably at least one IL-1β-D145K mutein antigen, wherein said antigen comprises at least one second attachment site; wherein (a) and (b) are linked through said at least one first and said at least one second attachment site, typically and preferably via at least one covalent non-peptide bond, and wherein said modified VLP of CMV comprises at least one chimeric CMV polypeptide, wherein said at least one chimeric CMV polypeptide comprises, preferably consists of, (i) a CMV polypeptide, wherein said CMV polypeptide comprises a coat protein of CMV or an amino acid sequence having a sequence identity of at least 75% with SEQ ID NO: 45; and (ii) a polypeptide comprising, preferably consisting of, a stretch of consecutive negative amino acids, wherein said negative amino acids are independently selected from aspartic acid or glutamic acid, and wherein said polypeptide is inserted between any amino acid residue of said CMV polypeptide corresponding to any amino acid residue between position 75 and position 85 of SEQ ID NO:45.

In a very preferred embodiment, said at least one first attachment site is not comprised or is not part of the polypeptide comprising said stretch of consecutive negative amino acids. In a very preferred embodiment, all of said first attachments sites are not comprised or are not part of the polypeptide comprising said stretch of consecutive negative amino acids. In a very preferred embodiment, said at least one first attachment site is not comprised or is not part of the stretch of consecutive negative amino acids. In a very preferred embodiment, all of said first attachments sites are not comprised or are not part of the stretch of consecutive negative amino acids. In a very preferred embodiment, said first attachment site and said second attachment site are linked solely via one or more covalent bonds. In a very preferred embodiment, said at least one antigen is linked to said modified VLP of CMV solely via one or more covalent bonds. In a very preferred embodiment, all of said antigens are linked to said modified VLP of CMV solely via one or more covalent bonds.

In a further preferred embodiment, said first attachment site is linked to said second attachment site via at least one covalent non-peptide bond. In a further preferred embodiment, all of said first attachment sites are linked to said second attachment sites via at least one covalent non-peptide bond. In a further very preferred embodiment, said first attachment site is an amino group, preferably an amino group of a lysine. In a further very preferred embodiment, all of said first attachment sites are an amino group, preferably an amino group of a lysine.

Thus, in a further aspect, the present invention provides a composition, preferably a veterinary composition, comprising
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of, the amino acid sequence of SEQ ID NO: 10, SEQ ID NO:11 or SEQ ID NO:12;
(b) at least one antigen, wherein said antigen comprises at least one second attachment site, and wherein said antigen is an interleukin 1β D145X mutein (IL-1β-D145X mutein) antigen, preferably an interleukin 1β D145K mutein (IL-1β-D145K mutein) antigen; and

wherein (a) and (b) are linked through said at least one first and said at least one second attachment site, preferably via at least one covalent non-peptide bond; and wherein said at least one first attachment site is not comprised or is not part of the polypeptide comprising said stretch of consecutive negative amino acids; and wherein preferably said first attachment sites are an amino group, hereby preferably an amino group of a lysine, and wherein further preferably the second attachment sites are a sulfhydryl group, preferably a sulfhydryl group of a cysteine residue or a sulfhydryl group that has been chemically attached to the IL-1β-D145X mutein antigen, preferably IL-1β-D145K mutein antigen.

In a very preferred embodiment, said chimeric CMV polypeptide comprises the amino acid sequence of SEQ ID NO:10. In a very preferred embodiment, said chimeric CMV polypeptide comprises the amino acid sequence of SEQ ID NO:11. In a very preferred embodiment, said chimeric CMV polypeptide comprises the amino acid sequence of SEQ ID NO: 12. In a very preferred embodiment, said chimeric CMV polypeptide consists of the amino acid sequence of SEQ ID NO:10, SEQ ID NO:11 or SEQ ID NO:12. In a very preferred embodiment, said chimeric CMV polypeptide consists of the amino acid sequence of SEQ ID NO:10. In a very preferred embodiment, said chimeric CMV polypeptide consists of the amino acid sequence of SEQ ID NO: 11. In a very preferred embodiment, said chimeric CMV polypeptide consists of the amino acid sequence of SEQ ID NO:12.

Attachment between modified virus-like particles and antigens by way of disulfide bonds are typically labile, in particular, to sulfhydryl-moiety containing molecules, and are, furthermore, less stable in serum than, for example, thioether attachments (Martin F J. and Papahadjopoulos D. (1982) J. Biol. Chem. 257:286-288). Therefore, in a further very preferred embodiment of the present invention, the association or linkage of the modified VLP of CMV and the at least one antigen does not comprise a disulfide bond. Further preferred hereby, the at least one second attachment site comprise, or preferably is, a sulfhydryl group. Preferably, all of said second attachment sites comprise, or preferably are, a sulfhydryl group. In a further preferred embodiment, said at least one first attachment site is not or does not comprise a sulfhydryl group. In a further preferred embodiment, all of said first attachment sites are not or do not comprise a sulfhydryl group. In a preferred embodiment, said at least one first attachment site is not or does not comprise a sulfhydryl group of a cysteine. In a preferred embodiment, all of said first attachment sites are not or do not comprise a sulfhydryl group of a cysteine. In a further very preferred embodiment said second attachment site is a sulfhydryl group, preferably a sulfhydryl group of a cysteine. In a further very preferred embodiment, all of said second attachment sites are a sulfhydryl group, preferably a sulfhydryl group of a cysteine.

In a very preferred embodiment, the at least one first attachment site is an amino group, preferably an amino group of a lysine residue and the at least one second attachment site is a sulfhydryl group, preferably a sulfhydryl group of a cysteine residue or a sulfhydryl group that has been chemically attached to the antigen. In a very preferred embodiment, all of said first attachment sites are an amino group, preferably an amino group of a lysine residue and all of said second attachment sites are a sulfhydryl group, preferably a sulfhydryl group of a cysteine residue or a sulfhydryl group that has been chemically attached to the antigen. In a further preferred embodiment only one of said second attachment sites associates with said first attachment site through at least one non-peptide covalent bond leading to a single and uniform type of binding of said antigen to said modified VLP of CMV, wherein said only one second attachment site that associates with said first attachment site is a sulfhydryl group, and wherein said antigen and said modified VLP of CMV interact through said association to form an ordered and repetitive antigen array.

In one preferred embodiment of the invention, the antigen is linked to the modified VLP of CMV by way of chemical cross-linking, typically and preferably by using a heterobifunctional cross-linker. Thus, in a preferred embodiment, the IL-1β-D145X mutein antigen, preferably IL-1β-D145K mutein antigen, is linked to the modified VLP of CMV by way of chemical cross-linking, typically and preferably by way of a heterobifunctional cross-linker through said at least one first and said at least one second attachment site via at least one covalent non-peptide bond. In preferred embodiments, the hetero-bifunctional cross-linker contains a functional group which can react with the preferred first attachment sites, preferably with the amino group, more preferably with the amino groups of lysine residue(s) of the modified VLP of CMV, and a further functional group which can react with the preferred second attachment site, i.e. a sulfhydryl group, preferably of cysteine(s) residue inherent of, or artificially added to the antigen, and optionally also made available for reaction by reduction. Several hetero-bifunctional cross-linkers are known to the art. These include the preferred cross-linkers succinimidyl-6-(b-maleimidopropionamide) hexanoate (SMPH) (Pierce), Sulfo-MBS, Sulfo-EMCS, Sulfo-GMBS, Sulfo-SIAB, Sulfo-SMPB, Sulfo-SMCC, Sulfo-KMUS SVSB, SIA, and other cross-linkers available for example from the Pierce Chemical Company, and having one functional group reactive towards amino groups and one functional group reactive towards sulfhydryl groups. The above mentioned cross-linkers all lead to formation of an amide bond after reaction with the amino group and a thioether linkage with the sulfhydryl groups. In a very preferred embodiment, said hetero-bifunctional cross-linker is SMPH. Thus, in a preferred embodiment, the IL-1β-D145X mutein antigen, preferably IL-1β-D145K mutein antigen, is linked to the modified VLP of CMV by way of chemical cross-linking, typically and preferably by way of a heterobifunctional cross-linker through said at least one first and said at least one second attachment site via at least one covalent non-peptide bond, and wherein said hetero-bifunctional cross-linker is SMPH. Another class of cross-linkers suitable in the practice of the invention is characterized by the introduction of a disulfide linkage between the antigen and the modified VLP upon coupling. Preferred cross-linkers belonging to this class include, for example, SPDP and Sulfo-LC-SPDP (Pierce).

Thus, in a further aspect, the present invention provides a composition, preferably a veterinary composition, comprising
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of, the amino acid sequence of SEQ ID NO: 10, SEQ ID NO:11 or SEQ ID NO:12;
(b) at least one antigen, wherein said antigen comprises at least one second attachment site, and wherein said antigen is an interleukin 1β D145X mutein (IL-1β-D145X mutein) antigen, preferably an interleukin 1β D145K mutein (IL-1β-D145K mutein) antigen; and

wherein (a) and (b) are linked by way of a heterobifunctional cross-linker through said at least one first and said at least one second attachment site via at least one covalent non-peptide bond, preferably wherein said hetero-bifunctional cross-linker is SMPH, and wherein said at least one first attachment site is not comprised or is not part of the polypeptide comprising said stretch of consecutive negative amino acids, and wherein said at least one first attachment site is an amino group, hereby preferably an amino group of a lysine, and wherein the at least one second attachment site is a sulfhydryl group, hereby preferably a sulfhydryl group of a cysteine residue or a sulfhydryl group that has been chemically attached to the IL-1β-D145X mutein antigen, preferably IL-1β-D145K mutein antigen.

In a very preferred embodiment, said chimeric CMV polypeptide comprises the amino acid sequence of SEQ ID NO: 10. In a very preferred embodiment, said chimeric CMV polypeptide comprises the amino acid sequence of SEQ ID NO:11. In a very preferred embodiment, said chimeric CMV polypeptide comprises the amino acid sequence of SEQ ID NO: 12. In a very preferred embodiment, said chimeric CMV polypeptide consists of the amino acid sequence of SEQ ID NO:10, SEQ ID NO:11 or SEQ ID NO:12. In a very preferred embodiment, said chimeric CMV polypeptide consists of the amino acid sequence of SEQ ID NO: 10. In a very preferred embodiment, said chimeric CMV polypeptide consists of the amino acid sequence of SEQ ID NO: 11. In a very preferred embodiment, said chimeric CMV polypeptide consists of the amino acid sequence of SEQ ID NO:12.

Linking of the antigen to the modified VLP of CMV by using a hetero-bifunctional cross-linker allows linking of the antigen to the modified VLP of CMV in an oriented fashion. Other methods of linking the antigen to the modified VLP of CMV include methods wherein the antigen is cross-linked to the modified VLP of CMV, using the carbodiimide EDC, and NHS. The antigen may also be first thiolated through reaction, for example with SATA, SATP or iminothiolane. The antigen, after deprotection if required, may then be coupled to the modified VLP of CMV as follows. After separation of the excess thiolation reagent, the antigen is reacted with the modified VLP of CMV, previously activated with a hetero-bifunctional cross-linker comprising a cysteine reactive moiety, and therefore displaying at least one or several functional groups reactive towards cysteine residues, to which the thiolated antigen can react, such as described above. Optionally, low amounts of a reducing agent are included in the reaction mixture. In further methods, the antigen is attached to the modified VLP of CMV, using a homo-bifunctional cross-linker such as glutaraldehyde, DSG, BM[PEO]4, BS3, (Pierce) or other known homo-bifunctional cross-linkers with functional groups reactive towards amine groups or carboxyl groups of the modified VLP.

In very preferred embodiments of the invention, the antigen is linked via a cysteine residue, having been added to either the N-terminus or the C-terminus of, or a natural cysteine residue within the antigen, to lysine residues of the modified VLP of CMV.

In a preferred embodiment, the composition of the invention further comprises a linker, wherein said linker associates said antigen with said second attachment site, and wherein preferably said linker comprises or alternatively consists of said second attachment site.

Engineering of a second attachment site onto the antigen is achieved by the association of a linker, typically and preferably containing at least one amino acid suitable as second attachment site according to the disclosures of this invention. Therefore, in a preferred embodiment of the present invention, a linker is associated to the antigen by way of at least one covalent bond, preferably, by at least one, preferably one peptide bond. Preferably, the linker comprises, or alternatively consists of, the second attachment site. In a further preferred embodiment, the linker comprises a sulfhydryl group, preferably of a cysteine residue. In another preferred embodiment, the linker comprises or is a cysteine residue. In a further preferred embodiment of the present invention, the linker consists of amino acids, wherein further preferably the linker consists at most 15 amino acids. In an again preferred embodiment of the invention, such amino acid linker contains 1 to 10 amino acids.

In again a further aspect, the present invention provides a composition, preferably a veterinary composition, comprising
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of, the amino acid sequence of SEQ ID NO: 10;
(b) at least one antigen, wherein said antigen comprises at least one second attachment site, and wherein said antigen is an interleukin 1β D145X mutein (IL-1β-D145X mutein) antigen, preferably an interleukin 1β D145K mutein (IL-1β-D145K mutein) antigen; and

wherein (a) and (b) are linked by way of a heterobifunctional cross-linker through said at least one first and said at least one second attachment site via at least one covalent non-peptide bond, wherein said hetero-bifunctional cross-linker is SMPH, and wherein said at least one first attachment site is not comprised or is not part of the polypeptide comprising said stretch of consecutive negative amino acids, and wherein said at least one first attachment site is an amino group, hereby preferably an amino group of a lysine, and wherein the at least one second attachment site is a sulfhydryl group, hereby preferably a sulfhydryl group that has been chemically attached to the IL-1β-D145X mutein antigen, preferably IL-1β-D145K mutein antigen.

In a very preferred embodiment, said chimeric CMV polypeptide consists of the amino acid sequence of SEQ ID NO:10. In a very preferred embodiment, said modified VLP of CMV comprises 180 identical chimeric CMV polypeptides, wherein said chimeric CMV polypeptide comprises, preferably consists of, the amino acid sequence of SEQ ID NO:10. In a very preferred embodiment, said modified VLP of CMV comprises 180 identical chimeric CMV polypeptides, wherein said chimeric CMV polypeptide consists of the amino acid sequence of SEQ ID NO:10.

In another preferred embodiment, said antigen is an interleukin 1β D145X mutein (IL-1β-D145X mutein) antigen, wherein the mutation at the position corresponding to position 145 of reference sequence SEQ ID NO:38 is a replacement of the amino acid aspartic acid (D) by an amino acid X, wherein X is selected from the group consisting of lysine (K), tyrosine (Y), phenylalanine (F), asparagine (N) and arginine (R), preferably of lysine (K).

In another preferred embodiment, said antigen is an interleukin 1β D145X mutein (IL-1β-D145X mutein) antigen, wherein said IL-1β-D145X mutein antigen is selected from human IL-1β-D145X (hIL-1β-D145X), equine IL-1β-D145X (eIL-1β-D145X), bovine IL-1β-D145X (bIL-1β-D145X), ovine IL-1β-D145X (bIL-1β-D145X), caprine IL-1β-D145X (bIL-13-D145X), and porcine IL-1β-D145X (pIL-1β-D145X), wherein preferably X is selected from the group consisting of lysine (K), tyrosine (Y), phenylalanine (F), asparagine (N) and arginine (R), preferably of lysine (K).

In another preferred embodiment, said antigen is an interleukin 1β D145K mutein (IL-1β-D145K mutein) antigen, wherein the mutation at the position corresponding to position 145 of reference sequence SEQ ID NO:38 is a replacement of the amino acid aspartic acid (D) by a lysine (K) residue.

In another preferred embodiment, said antigen is an interleukin 1β D145K mutein (IL-1β-D145K mutein) antigen, wherein said IL-1β-D145K mutein antigen is selected from human IL-1β-D145K (hIL-1β-D145K), equine IL-1β-D145K (eIL-1β-D145K), bovine IL-1β-D145K (bIL-1β-D145K), ovine IL-1β-D145K (bIL-1β-D145K), caprine IL-1β-D145K (bIL-1β-D145K), and porcine IL-1β-D145K (pIL-1β-D145K), which have a lysine (K) residue at the position corresponding to position 145 of SEQ ID NO:38.

In a further preferred embodiment, said IL-1β-D145X mutein antigen, preferably IL-1β-D145K mutein antigen, comprises a polyhistidine-tag of at least two consecutive and at most 12 consecutive histidine residues, preferably C-terminally or N-terminally positioned of the IL-1β-D145X mutein antigen, preferably IL-1β-D145K mutein antigen. In a further preferred embodiment, said IL-1β-D145X mutein antigen, preferably IL-1β-D145K mutein antigen, comprises a polyhistidine-tag of at least 2, 3, 4, 5, 6, 7, 8, 9, 10, 11 or 12 consecutive histidine residues, preferably C-terminally or N-terminally positioned of the IL-1β-D145X mutein antigen, preferably IL-1β-D145K mutein antigen. In a further preferred embodiment, said IL-1β-D145X mutein antigen, preferably IL-1β-D145K mutein antigen, comprises a polyhistidine-tag of 4, 6, 8 or 10 consecutive histidine residues, preferably C-terminally or N-terminally positioned of the IL-1β-D145X mutein antigen, preferably IL-1β-D145K mutein antigen. In a further preferred embodiment, said IL-1β-D145X mutein antigen, preferably IL-1β-D145K mutein antigen comprises a polyhistidine-tag of 4 consecutive histidine residues, preferably C-terminally positioned of the IL-1β-D145X mutein antigen, preferably IL-1β-D145K mutein antigen. In a further preferred embodiment, said IL-1β-D145X mutein antigen, preferably IL-1β-D145K mutein antigen, comprises a polyhistidine-tag of 4 consecutive histidine residues, preferably N-terminally positioned of the IL-1β-D145X mutein antigen, preferably IL-1β-D145K mutein antigen. In a further preferred embodiment, said IL-1β-D145X mutein antigen, preferably IL-1β-D145K mutein antigen, comprises a polyhistidine-tag of 6 consecutive histidine residues consisting of SEQ ID NO:31, preferably C-terminally positioned of the IL-1β-D145X mutein antigen, preferably IL-1β-D145K mutein antigen. In a further preferred embodiment, said IL-1β-D145X mutein antigen, preferably IL-1β-D145K mutein antigen, comprises a polyhistidine-tag of 6 consecutive histidine residues consisting of SEQ ID NO: 31, preferably N-terminally positioned of the IL-1β-D145X mutein antigen, preferably IL-1β-D145K mutein antigen. In a further preferred embodiment, said IL-1β-D145X mutein antigen, preferably IL-1β-D145K mutein antigen, comprises a polyhistidine-tag of 8 consecutive histidine residues, preferably C-terminally positioned of the IL-1β-D145X mutein antigen, preferably IL-1β-D145K mutein antigen. In a further preferred embodiment, said IL-1β-D145X mutein antigen, preferably IL-1β-D145K mutein antigen, comprises a polyhistidine-tag of 8 consecutive histidine residues, preferably N-terminally positioned of the IL-1β-D145X mutein antigen, preferably IL-1β-D145K mutein antigen. In a further preferred embodiment, said IL-1β-D145X mutein antigen, preferably IL-1β-D145K mutein antigen, comprises a polyhistidine-tag of 10 consecutive histidine residues, preferably C-terminally or N-terminally positioned of the IL-1β-D145X mutein antigen, preferably IL-1β-D145K mutein antigen.

In an alternative embodiment, said antigen is a human IL-1β-D145X mutein antigen, preferably a human IL-1β-D145K mutein antigen. In a further embodiment, said antigen comprises, or preferably consists of, (i) a polypeptide having an amino acid sequence having the sequence of SEQ ID NO:60 or (ii) polypeptide having an amino acid sequence having a sequence identity of at least 90%, at least 92%, at least 95%, at least 98%, or at least 99% with SEQ ID NO:60 and preferably having an amino acid X at the position corresponding to position 145 of reference sequence SEQ ID NO:38, wherein X is selected from the group consisting of lysine (K), tyrosine (Y), phenylalanine (F), asparagine (N), and arginine (R). In a further embodiment, said antigen comprises SEQ ID NO:60. In a further, said antigen consists of SEQ ID NO:60.

In a further embodiment, said antigen is an equine IL-1β-D145X mutein antigen, preferably an equine IL-1β-D145K mutein antigen. In a further embodiment, said antigen is a bovine IL-1β-D145X mutein antigen, preferably a bovine IL-1β-D145K mutein antigen. In a further embodiment, said antigen is a porcine IL-1β-D145X mutein antigen, preferably a porcine IL-1β-D145K mutein antigen. In a further embodiment, said antigen is an ovine IL-1β-D145X mutein antigen, preferably a ovine IL-1β-D145K mutein antigen. In a further embodiment, said antigen is a caprine IL-1β-D145X mutein antigen, preferably a caprine IL-1β-D145K mutein antigen.

Without being bound, the inventors believe that undesired aggregation and formation of aggregated conjugated CMV VLPs can in particular be reduced and avoided for antigens having a higher isoelectric point, and thus for antigens, which under the conditions used for conjugation would have an overall positive charge. Thus, in a preferred embodiment, said IL-1β-D145X antigen, preferably IL-1β-D145K antigen, has an isoelectric point of above 6.5. In a preferred embodiment, said IL-1β-D145X antigen, preferably IL-1β-D145K antigen, has an isoelectric point above 6.5 and below 13.0, preferably below 12.5, and further preferably below 12.0. In a preferred embodiment, said IL-1β-D145X antigen, preferably IL-1β-D145K antigen, has an isoelectric point above 6.5, as determined by the ExPASy Compute pI/MW tool described by Gasteiger et al (Gasteiger, E., Hoogland, C., Gattiker, A., Duvaud, S., Wilkins, M. R., Appel, R. D., & Bairoch, A., Protein Identification and Analysis Tools on the ExPASy Server, (In) John M. Walker (ed): The Proteomics Protocols Handbook, Humana Press (2005). In a preferred embodiment, said IL-1β-D145X antigen, preferably IL-1β-D145K antigen, has an isoelectric point above 6.5 and below 13.0, preferably below 12.5, and further preferably below 12.0, as determined by the ExPASy Compute pI/MW tool described by Gasteiger et al (Gasteiger, E., Hoogland, C., Gattiker, A., Duvaud, S., Wilkins, M. R., Appel, R. D., & Bairoch, A., Protein Identification and Analysis Tools on the ExPASy Server, (In) John M. Walker (ed): The Proteomics Protocols Handbook, Humana Press (2005). In a preferred embodiment, said IL-1β-D145X antigen, preferably IL-1β-D145K antigen, has an isoelectric point of above 6.6, 6.7, 6.8 or 6.9. In a preferred embodiment, said IL-1β-D145X antigen, preferably IL-1β-D145K antigen, has an isoelectric point above 6.6, 6.7, 6.8 or 6.9 and below 13.0, preferably below 12.5, and further preferably below 12.0. In a preferred embodiment, said IL-1β-D145X antigen, preferably IL-1β-D145K antigen, has an isoelectric point of above 6.6, 6.7, 6.8 or 6.9, as determined by the ExPASy Compute pI/MW tool. In a preferred embodiment, said IL-1β-D145X antigen, preferably IL-1β-D145K antigen, has an isoelectric point above 6.6, 6.7, 6.8 or 6.9 and below 13.0, preferably below 12.5, and further preferably below 12.0, as determined by the ExPASy Compute pI/MW tool. In a preferred embodiment, said IL-1β-D145X antigen, preferably IL-1β-D145K antigen, has an isoelectric point of equal to or above 7.0. In a preferred embodiment, said IL-1β-D145X antigen, preferably IL-1β-D145K antigen, has an isoelectric point equal or above 7.0 and below 13.0, preferably below 12.5, and further preferably below 12.0. In a preferred embodiment, said IL-1β-D145X antigen, preferably IL-1β-D145K antigen, has an isoelectric point equal to or above 7.0, as determined by the ExPASy Compute pI/MW tool. In a preferred embodiment, said IL-1β-D145X antigen, preferably IL-1β-D145K antigen, has an isoelectric point equal or above 7.0 and below 13.0, preferably below 12.5, and further preferably below 12.0, as determined by the ExPASy Compute pI/MW tool. In a preferred embodiment, said IL-1β-D145X antigen, preferably IL-1β-D145K antigen, has an isoelectric point equal to or above 7.1, 7.2, 7.3 or 7.4. In a preferred embodiment, said IL-1β-D145X antigen, preferably IL-1β-D145K antigen, has an isoelectric point equal to or above 7.1, 7.2, 7.3 or 7.4 and below 13.0, preferably below 12.5, and further preferably below 12.0. In a preferred embodiment, said IL-1β-D145X antigen, preferably IL-1β-D145K antigen, has an isoelectric point equal to or above 7.1, 7.2, 7.3 or 7.4, as determined by the ExPASy Compute pI/MW tool. In a preferred embodiment, said IL-1β-D145X antigen, preferably IL-1β-D145K antigen, has an isoelectric point equal to or above 7.1, 7.2, 7.3 or 7.4 and of below 13.0, preferably below 12.5, and further preferably below 12.0, as determined by the ExPASy Compute pI/MW tool. In a preferred embodiment, said IL-1β-D145X antigen, preferably IL-1β-D145K antigen, has an isoelectric point equal to or above 7.5. In a preferred embodiment, said IL-1β-D145X antigen, preferably IL-1β-D145K antigen, has an isoelectric point equal to or above 7.5 and below 13.0, preferably below 12.5, and further preferably below 12.0. In a preferred embodiment, said IL-1β-D145X antigen, preferably IL-1β-D145K antigen, has an isoelectric point equal to or above 7.5, as determined by the ExPASy Compute pI/MW tool. In a preferred embodiment, said antigen has an isoelectric point equal to or above 7.5 and below 13.0, preferably below 12.5, and further preferably below 12.0, as determined by the ExPASy Compute pI/MW tool. In a preferred embodiment, said IL-1β-D145X antigen, preferably IL-1β-D145K antigen, has an isoelectric point of equal or above 7.6, 7.7, 7.8 or 7.9. In a preferred embodiment, said IL-1β-D145X antigen, preferably IL-1β-D145K antigen, has an isoelectric point equal or above 7.6, 7.7, 7.8 or 7.9 and below 13.0, preferably below 12.5, and further preferably below 12.0. In a preferred embodiment, said IL-1β-D145X antigen, preferably IL-1β-D145K antigen, has an isoelectric point of equal or above 7.6, 7.7, 7.8 or 7.9, as determined by the ExPASy Compute pI/MW tool. In a preferred embodiment, said IL-1β-D145X antigen, preferably IL-1β-D145K antigen, has an isoelectric point equal or above 7.6, 7.7, 7.8 or 7.9 and below 13.0, preferably below 12.5, and further preferably below 12.0, as determined by the ExPASy Compute pI/MW tool. In a preferred embodiment, said IL-1β-D145X antigen, preferably IL-1β-D145K antigen, has an isoelectric point of equal or above 8.0. In a preferred embodiment, said IL-1β-D145X antigen, preferably IL-1β-D145K antigen, has an isoelectric point equal or above 8.0. and below 13.0, preferably below 12.5, and further preferably below 12.0. In a preferred embodiment, said IL-1β-D145X antigen, preferably IL-1β-D145K antigen, has an isoelectric point of equal or above 8.0, as determined by the ExPASy Compute pI/MW tool. In a preferred embodiment, said IL-1β-D145X antigen, preferably IL-1β-D145K antigen, has an isoelectric point equal or above 8.0 and below 13.0, preferably below 12.5, and further preferably below 12.0, as determined by the ExPASy Compute pI/MW tool. In a preferred embodiment, said IL-1β-D145X antigen, preferably IL-1β-D145K antigen, has an isoelectric point of equal or above 8.1, 8.2, 8.3 or 8.4. In a preferred embodiment, said IL-1β-D145X antigen, preferably IL-1β-D145K antigen, has an isoelectric point equal or above 8.1, 8.2, 8.3 or 8.4. and below 13.0, preferably below 12.5, and further preferably below 12.0. In a preferred embodiment, said IL-1β-D145X antigen, preferably IL-1β-D145K antigen, has an isoelectric point of equal or above 8.1, 8.2, 8.3 or 8.4, as determined by the ExPASy Compute pI/MW tool. In a preferred embodiment, said IL-1β-D145X antigen, preferably IL-1β-D145K antigen, has an isoelectric point equal to or above 8.1, 8.2, 8.3 or 8.4 and below 13.0, preferably below 12.5, and further preferably below 12.0, as determined by the ExPASy Compute pI/MW tool. In a preferred embodiment, said IL-1β-D145X antigen, preferably IL-1β-D145K antigen, has an isoelectric point equal to or above 8.5. In a preferred embodiment, said IL-1β-D145X antigen, preferably IL-1β-D145K antigen, has an isoelectric point equal to or above 8.5. and below 13.0, preferably below 12.5, and further preferably below 12.0. In a preferred embodiment, said IL-1β-D145X antigen, preferably IL-1β-D145K antigen, has an isoelectric point of equal or above 8.5, as determined by the ExPASy Compute pI/MW tool. In a preferred embodiment, said IL-1β-D145X antigen, preferably IL-1β-D145K antigen, has an isoelectric point equal to or above 8.5 and below 13.0, preferably below 12.5, and further preferably below 12.0, as determined by the ExPASy Compute pI/MW tool.

In a very preferred embodiment, a D/E polypeptide comprising a stretch of consecutive negative amino acids comprises SEQ ID NO:49, SEQ ID NO:50 or SEQ ID NO: 51. In a further very preferred embodiment, said D/E polypeptide consists of SEQ ID NO: 49, SEQ ID NO:50 or SEQ ID NO:51. In a further very preferred embodiment, said D/E polypeptide comprises SEQ ID NO:49. In a further very preferred embodiment, said D/E polypeptide comprises SEQ ID NO:50. In a further very preferred embodiment, said D/E polypeptide comprises SEQ ID NO:51. In a further very preferred embodiment, said D/E polypeptide consists of SEQ ID NO:49. In a further very preferred embodiment, said D/E polypeptide consists of SEQ ID NO:50. In a further very preferred embodiment, said D/E polypeptide consists of SEQ ID NO:51.

In a very preferred embodiment, said CMV polypeptide comprises the amino acid sequence of SEQ ID NO:5, SEQ ID NO:45 or SEQ ID NO:52, wherein said polypeptide comprising said stretch of consecutive negative amino acids is inserted between amino acid residues of position 88 (Ser) and position 89 (Thr) of said SEQ ID NO:5, between amino acid residues of position 84 (Ser) and position 85 (Thr) of SEQ ID NO:45, or between amino acid residues of position 86 (Ser) and position 87 (Thr) of SEQ ID NO:52. In a very preferred embodiment, said CMV polypeptide consists of the amino acid sequence of SEQ ID NO:5, SEQ ID NO:45 or SEQ ID NO:52, wherein said polypeptide comprising said stretch of consecutive negative amino acids is inserted between amino acid residues of position 88 and position 89 of said SEQ ID NO:5, between amino acid residues of position 84 and position 85 of SEQ ID NO:45, or between amino acid residues of position 86 and position 87 of SEQ ID NO: 52. In a very preferred embodiment, said CMV polypeptide comprises the amino acid sequence of SEQ ID NO:5, wherein said polypeptide is inserted between amino acid residues of position 88 and position 89 of SEQ ID NO:5. In a very preferred embodiment, said CMV polypeptide comprises the amino acid sequence of SEQ ID NO:45, wherein said polypeptide is inserted between amino acid residues of position 84 and position 85 of SEQ ID NO: 45. In a very preferred embodiment, said CMV polypeptide comprises the amino acid sequence of SEQ ID NO:52, wherein said polypeptide is inserted between amino acid residues of position 86 and position 87 of SEQ ID NO:52. In a very preferred embodiment, said CMV polypeptide consists of the amino acid sequence of SEQ ID NO:5, wherein said polypeptide is inserted between amino acid residues of position 88 and position 89 of SEQ ID NO: 5. In a very preferred embodiment, said CMV polypeptide consists of the amino acid sequence of SEQ ID NO:45, wherein said polypeptide is inserted between amino acid residues of position 84 and position 85 of SEQ ID NO:45. In a very preferred embodiment, said CMV polypeptide consists of the amino acid sequence of SEQ ID NO:52, wherein said polypeptide is inserted between amino acid residues of position 86 and position 87 of SEQ ID NO: 52.

In a very preferred embodiment, said chimeric CMV polypeptide comprises the amino acid sequence of SEQ ID NO:5, SEQ ID NO:45 or SEQ ID NO:52, wherein said polypeptide comprising said stretch of consecutive negative amino acids is inserted between amino acid residues of position 88 (Ser) and position 89 (Thr) of said SEQ ID NO:5, between amino acid residues of position 84 (Ser) and position 85 (Thr) of SEQ ID NO:45, or between amino acid residues of position 86 (Ser) and position 87 (Thr) of SEQ ID NO:52. In a very preferred embodiment, said chimeric CMV polypeptide consists of the amino acid sequence of SEQ ID NO:5, SEQ ID NO:45 or SEQ ID NO:52, wherein said polypeptide comprising said stretch of consecutive negative amino acids is inserted between amino acid residues of position 88 and position 89 of said SEQ ID NO:5, between amino acid residues of position 84 and position 85 of SEQ ID NO:45, or between amino acid residues of position 86 and position 87 of SEQ ID NO:52. In a very preferred embodiment, said chimeric CMV polypeptide comprises the amino acid sequence of SEQ ID NO:5, wherein said polypeptide is inserted between amino acid residues of position 88 and position 89 of SEQ ID NO:5. In a very preferred embodiment, said chimeric CMV polypeptide comprises the amino acid sequence of SEQ ID NO:45, wherein said polypeptide is inserted between amino acid residues of position 84 and position 85 of SEQ ID NO:45. In a very preferred embodiment, said chimeric CMV polypeptide comprises the amino acid sequence of SEQ ID NO:52, wherein said polypeptide is inserted between amino acid residues of position 86 and position 87 of SEQ ID NO:52. In a very preferred embodiment, said chimeric CMV polypeptide consists of the amino acid sequence of SEQ ID NO:5, wherein said polypeptide is inserted between amino acid residues of position 88 and position 89 of SEQ ID NO:5. In a very preferred embodiment, said chimeric CMV polypeptide consists of the amino acid sequence of SEQ ID NO:45, wherein said polypeptide is inserted between amino acid residues of position 84 and position 85 of SEQ ID NO:45. In a very preferred embodiment, said chimeric CMV polypeptide consists of the amino acid sequence of SEQ ID NO:52, wherein said polypeptide is inserted between amino acid residues of position 86 and position 87 of SEQ ID NO: 52.

In a very preferred embodiment, said CMV polypeptide comprises the amino acid sequence of SEQ ID NO:5 and said polypeptide comprising said stretch of consecutive negative amino acids is inserted between amino acid residues 88 (Ser) and amino acid residue 89 (Thr) of SEQ ID NO:5, and said polypeptide comprising said stretch of consecutive negative amino acids further comprises a first and a second amino acid linker, wherein said first and said second amino acid linker is independently a glycine-serine linker (GS-linker) comprising at least one glycine and at least one serine, or an amino acid linker (GS*-linker) comprising at least one Gly, at least one Ser, and at least one amino acid selected from Thr, Ala, Lys, and Cys, wherein said first and/or said second amino acid linker has a Gly-Ser sequence at its N-terminus.

In a very preferred embodiment, said chimeric CMV polypeptide comprises the amino acid sequence of SEQ ID NO:5 and said polypeptide comprising said stretch of consecutive negative amino acids is inserted between amino acid residues 88 (Ser) and amino acid residue 89 (Thr) of SEQ ID NO:5, and said polypeptide comprising said stretch of consecutive negative amino acids further comprises a first and a second amino acid linker, wherein said first and said second amino acid linker is independently a glycine-serine linker (GS-linker) comprising at least one glycine and at least one serine or an amino acid linker (GS*-linker) comprising at least one Gly, at least one Ser, and at least one amino acid selected from Thr, Ala, Lys, and Cys, wherein said first and/or said second amino acid linker has a Gly-Ser sequence at its N-terminus.

In a very preferred embodiment, said chimeric CMV polypeptide comprises the amino acid sequence of SEQ ID NO:10, SEQ ID NO:11 or SEQ ID NO:12. In a very preferred embodiment, said chimeric CMV polypeptide comprises the amino acid sequence of SEQ ID NO:10. In a very preferred embodiment, said chimeric CMV polypeptide comprises the amino acid sequence of SEQ ID NO: 11. In a very preferred embodiment, said chimeric CMV polypeptide comprises the amino acid sequence of SEQ ID NO: 12. In a very preferred embodiment, said chimeric CMV polypeptide consists of the amino acid sequence of SEQ ID NO:10, SEQ ID NO:11 or SEQ ID NO:12. In a very preferred embodiment, said chimeric CMV polypeptide consists of the amino acid sequence of SEQ ID NO: 10. In a very preferred embodiment, said chimeric CMV polypeptide consists of the amino acid sequence of SEQ ID NO:11. In a very preferred embodiment, said chimeric CMV polypeptide consists of the amino acid sequence of SEQ ID NO:12.

In a very preferred embodiment, said modified VLP of CMV comprises 180 copies of said chimeric CMV polypeptide comprising the amino acid sequence of SEQ ID NO: 10, SEQ ID NO:11 or SEQ ID NO:12. In a very preferred embodiment, said modified VLP of CMV comprises 180 copies of said chimeric CMV polypeptide comprising the amino acid sequence of SEQ ID NO:10. In a very preferred embodiment, said modified VLP of CMV comprises 180 copies of said chimeric CMV polypeptide comprising the amino acid sequence of SEQ ID NO:11. In a very preferred embodiment, said modified VLP of CMV comprises 180 copies of said chimeric CMV polypeptide comprising the amino acid sequence of SEQ ID NO:12. In a very preferred embodiment, said modified VLP of CMV comprises 180 copies of said chimeric CMV polypeptide consisting of the amino acid sequence of SEQ ID NO:10, SEQ ID NO:11 or SEQ ID NO:12. In a very preferred embodiment, said modified VLP of CMV comprises 180 copies of said chimeric CMV polypeptide consisting of the amino acid sequence of SEQ ID NO:10. In a very preferred embodiment, said modified VLP of CMV comprises 180 copies of said chimeric CMV polypeptide consisting of the amino acid sequence of SEQ ID NO:11. In a very preferred embodiment, said modified VLP of CMV comprises 180 copies of said chimeric CMV polypeptide consisting of the amino acid sequence of SEQ ID NO:12.

The modified VLPs of the invention can be prepared in prokaryotic or eukaryotic expression systems. Preferred systems are E. coli, yeast, insect cells as well as mammalian cell lines. Very preferred said modified VLP of CMV or said VLP of CMV is obtained by expression of said chimeric CMV polypeptide in E. coli, and wherein preferably said expression is effected at temperatures of between 10° C. to 35° C.

Therefore, in another aspect, the present invention provides for composition comprising (a) a modified virus-like particle (VLP) of cucumber mosaic virus (CMV) comprising at least one chimeric CMV polypeptide, wherein said at least one chimeric CMV polypeptide comprises, preferably consists of (i) a CMV polypeptide, wherein said CMV polypeptide comprises a coat protein of CMV or an amino acid sequence having a sequence identity of at least 75% with SEQ ID NO:45; and (ii) a polypeptide comprising, preferably consisting of, a stretch of consecutive negative amino acids, wherein said negative amino acids are independently selected from aspartic acid or glutamic acid, wherein said polypeptide is inserted between any amino acid residue of said CMV polypeptide corresponding to any amino acid residue between position 75 and position 85 of SEQ ID NO: 45; and (b) at least one antigen, wherein said antigen comprises at least one second attachment site, and wherein said antigen is an interleukin 1β D145X mutein (IL-1β-D145X mutein) antigen, preferably an interleukin 1β D145K mutein (IL-1β-D145K mutein) antigen; and wherein (a) and (b) are linked through said at least one first and said at least one second attachment site via at least one covalent non-peptide bond, and wherein said modified VLP of CMV is obtained by expression of said chimeric CMV polypeptide in E. coli, and wherein preferably said expression is effected at temperatures of between 10° C. to 35° C.

In another aspect, the present invention provides for a process for producing the inventive composition comprising the purification of said modified virus-like particle (VLP) of cucumber mosaic virus (CMV) from a recombinant bacterial host expressing said modified VLP of CMV, wherein said modified VLP of CMV comprises at least one chimeric CMV polypeptide, wherein said at least one chimeric CMV polypeptide comprises, preferably consists of (i) a CMV polypeptide, wherein said CMV polypeptide comprises a coat protein of CMV or an amino acid sequence having a sequence identity of at least 75% with SEQ ID NO:45; and (ii) a polypeptide comprising, preferably consisting of, a stretch of consecutive negative amino acids, wherein said negative amino acids are independently selected from aspartic acid or glutamic acid, wherein said polypeptide is inserted between any amino acid residue of said CMV polypeptide corresponding to any amino acid residue between position 75 and position 85 of SEQ ID NO:45; and wherein the process comprises the steps of: (a) lysing said bacterial host; (b) clarifying the lysate obtained by said lysis; (c) purifying said modified VLP of CMV from the clarified lysate by anion exchange chromatography (AEX); wherein said steps are performed in the given order.

In a embodiment, said composition comprises an adjuvant. Typical and preferred adjuvants are mineral salts (e.g. Aluminium Hydroxide, Aluminium Phosphate), microcrystalline tyrosine, emulsions, microparticles, saponins (Quil A), cytokines, immune potentiators, microbial components/products, liposomes, complexes, and mucosal adjuvants which are known and as described such, and for example, in the Adjuvant Compendium NIAID and VAC (nih.gov) or by Aguilar et al, (Aguilar J C et al, 2007, Vaccine 25:3752-3762), Gerdts (Gerdts V, 2015, Berliner und Münchener Tierärztliche Wochenschrift 128:456-463) and Pasquale et al. (Pasquale et al. 2015, Vaccines 3:320-343). In a preferred embodiment, said composition comprises an adjuvant, wherein said adjuvant is aluminium hydroxide. In another preferred embodiment, said composition is devoid of an adjuvant.

In a further aspect, the present invention provides vaccines, preferably said vaccines are veterinary vaccines comprising, or alternatively consisting of, the inventive composition comprising said modified VLP of CMV and at least one interleukin 1β D145X (IL-1β-D145X) antigen, preferably interleukin 1β D145K (IL-1β-D145K) antigen as described herein. Encompassed are vaccines wherein said inventive composition comprise any one of the technical features disclosed herein, either alone or in any possible combination. In an embodiment, the vaccine further comprises an adjuvant. In an embodiment, said vaccine comprises an adjuvant, wherein said adjuvant is aluminium hydroxide. In a further embodiment the vaccine is devoid of an adjuvant. In a preferred embodiment said vaccine comprises an effective amount of the composition of the invention.

In a further aspect, the invention relates to a pharmaceutical composition comprising: (a) the inventive composition as described herein, or the vaccine of the invention as described herein; and (b) a pharmaceutically acceptable carrier, diluent and/or excipient. Said diluent includes sterile aqueous (e.g., physiological saline) or non-aqueous solutions and suspensions. Pharmaceutical compositions of the invention may be in a form which contain salts, buffers, adjuvants, or other substances which are desirable for improving the efficacy of the conjugate. In a preferred embodiment, said pharmaceutical composition comprises an effective amount of the vaccine of the invention. In an embodiment, said pharmaceutical composition comprises an adjuvant.

A further aspect of the present invention is a method of immunization comprising administering the inventive composition as described herein, the vaccine of the invention as described herein, or the pharmaceutical composition as described herein, to an animal, In a preferred embodiment said method comprises administering the inventive composition as described herein, the vaccine of the invention as described herein, or the pharmaceutical composition as described herein, to an animal. In a preferred embodiment said method comprises administering an effective amount of said inventive composition, said vaccine, or said pharmaceutical composition to said animal.

In a further aspect, the present invention provides the modified VLP of CMV as described herein, the inventive composition as described herein, the vaccine of the invention as described herein, or the pharmaceutical composition as described herein for use in a method of immunization an animal wherein said method comprises administering an effective amount of said modified VLP of CMV, said inventive composition, said vaccine, or said pharmaceutical composition to said animal.

A further aspect of the present invention is a method of inducing neutralizing antibodies against IL-1β in an animal comprising administering the inventive composition as described herein, the vaccine of the invention as described herein, or the pharmaceutical composition as described herein, to said animal. In a preferred embodiment said method comprises administering the inventive composition as described herein, the vaccine of the invention as described herein, or the pharmaceutical composition as described herein, to an animal. In a preferred embodiment said method comprises administering an effective amount of said inventive composition, said vaccine, or said pharmaceutical composition to said animal.

In a further aspect, the present invention provides the modified VLP of CMV as described herein, the inventive composition as described herein, the vaccine of the invention as described herein, or the pharmaceutical composition as described herein for use in a method of immunization an animal wherein said method comprises administering an effective amount of said modified VLP of CMV, said inventive composition, said vaccine, or said pharmaceutical composition to said animal.

The present invention further contemplates the following aspects and embodiments referred to as items:

1. A composition, optionally a veterinary composition, comprising:
(a) a modified VLP (virus-like particle) of CMV (cucumber mosaic virus), wherein said modified VLP of CMV comprises at least one first attachment site, and wherein said modified VLP of CMV comprises at least one chimeric CMV polypeptide, wherein said at least one chimeric CMV polypeptide comprises,
   (i) a first polypeptide, wherein said first polypeptide comprises (a) a coat protein of CMV or (b) an amino acid sequence having a sequence identity of at least 75% with SEQ ID NO:45; and
   (ii) a second polypeptide comprising, optionally consisting of, a stretch of consecutive negative amino acids, wherein said negative amino acids are independently selected from aspartic acid and/or glutamic acid, wherein said second polypeptide is inserted between any two adjacent amino acid residues of said first polypeptide, wherein said any two adjacent amino acid residues correspond to any two adjacent amino acid residues between position 75 and position 85 of SEQ ID NO:45;
(b) at least one antigen, wherein said antigen comprises (i) at least one second attachment site and (ii) at least one interleukin 1β D145X mutein (IL-1β-D145X mutein), wherein the IL-1β-D145X mutein comprises (A) a polypeptide having an amino acid sequence of an IL-1β polypeptide, optionally a mature IL-1β polypeptide, wherein the amino acid sequence of the IL-1β polypeptide is mutated via amino acid substitution to comprise an amino acid residue X at a position corresponding to position 145 of SEQ ID NO:38 or (B) a polypeptide having an amino acid having at least 90%, at least 91%, at least 92%, at least 93%, at least 94%, at least 95%, at least 96%, at least 97%, at least 98%, or at least 99% sequence identity to an amino acid sequence as defined in (A) and having an amino acid residue X at a position corresponding to position 145 of SEQ ID NO:38; and
wherein (a) and (b) are linked through the at least one first and said at least one second attachment site, preferably via at least one covalent non-peptide bond.

2. The composition of item 1, wherein (a) and (b) are linked through the at least one first and the at least one second attachment site via at least one covalent non-peptide bond; and wherein the at least one first attachment site is not comprised or is not part of the polypeptide comprising the stretch of consecutive negative amino acids; and wherein optionally the first attachment sites are an amino group, optionally an amino group of a lysine, and wherein further optionally the second attachment sites are a sulfhydryl group, optionally a sulfhydryl group of a cysteine residue or a sulfhydryl group that has been chemically attached to the antigen.

3. The composition of any one of items 1-2, wherein (a) and (b) are linked via a heterobifunctional cross-linker through said at least one first and said at least one second attachment site via at least one covalent non-peptide bond, optionally wherein said hetero-bifunctional cross-linker is SMPH, and wherein said at least one first attachment site is not comprised or is not part of the polypeptide comprising said stretch of consecutive negative amino acids, and wherein said at least one first attachment site is an amino group, optionally an amino group of a lysine, and wherein the at least one second attachment site is a sulfhydryl group, optionally a sulfhydryl group of a cysteine residue or a sulfhydryl group that has been chemically attached to the antigen.

4. The composition of item 1, wherein the first polypeptide (a) comprises SEQ ID NO: 45 or (b) comprises an amino acid sequence having sequence identity of at least 80%, at least 85%, at least 90%, at least 91%, at least 92%, at least 93%, at least 94%, at least 95%, at least at least 96%, or at least 97%, at least about 98%, or at least 99% to SEQ ID NO:45.

5. The composition of any one of items 1-4, wherein at least one of the at least one chimeric CMV polypeptide further comprises a T helper cell epitope.

6. The composition of item 5, wherein the T helper cell epitope replaces an N-terminal region of the first polypeptide, optionally wherein said replaced N-terminal region of said first polypeptide consists of 5 to 15 consecutive amino acids, 9 to 14 consecutive amino acids, 9 to 13 consecutive amino acids, 10 to 13 consecutive amino acids, 11 to 13 consecutive amino acids, 11 consecutive amino acids, 12 consecutive amino acids, or 13 consecutive amino acids.

7. The composition of item 6, wherein said replaced N-terminal region of said first polypeptide corresponds to amino acids 2-12 of SEQ ID NO:45.

8. The composition of item 7, wherein a methionine residue is inserted at the N-terminus of the T helper cell epitope.

9. The composition of any one of items 5-8, wherein one, two, or three additional amino acids are inserted between the N-terminal methionine and the T helper cell epitope.

10. The composition of any one of items 5-9, wherein T helper cell epitope is derived from tetanus toxin, comprises or consists of a PADRE sequence, comprises or consists of the amino acid sequence of SEQ ID NO:47, or comprises or consists of the amino acid sequence of SEQ ID NO:48.

11. The composition of any one of items 1-10, wherein the stretch of consecutive negative amino acids comprises or consists of SEQ ID NO:1 or SEQ ID NO:2.

12. The composition of any one of items 1-11, wherein the second polypeptide further comprises (i) a first amino acid linker and/or (ii) a second amino acid linker; wherein, if present, said first amino acid linker is positioned at the N-terminus of the stretch of consecutive negative amino acids, and, if present, the second amino acid linker is positioned at the C-terminus of said stretch of consecutive negative amino acids.

13. The composition of item 12, wherein said first and said second amino acid linker are each independently selected from the group consisting of (a) a polyglycine linker (G-linker) having an amino acid sequence (Gly)ₙ of a length of n=2-10; (b) a glycine-serine linker (GS-linker) comprising at least one glycine and at least one serine, wherein optionally said GS linker has an amino acid sequence of (GS)ᵣ(GₛS)ₜ(GS)ᵤ with r=0 or 1, s=1-5, t=1-5, and u=0 or 1; and (c) an amino acid linker (GS*-linker) comprising at least one Gly, at least one Ser, and at least one amino acid selected from Thr, Ala, Lys, and Cys.

14. The composition of item 12, wherein said first amino acid linker comprises, optionally consists of, SEQ ID NO:8 and/or said second amino acid linker comprises, optionally consists of, SEQ ID NO:4 or SEQ ID NO:9.

15. The composition of any one of items 1-14, wherein said second polypeptide comprises or consists of SEQ ID NO:49, SEQ ID NO:50, or SEQ ID NO:51.

16. The composition of any one of items 1-15, wherein said second polypeptide is inserted between the two adjacent amino acid residues of said first polypeptide that correspond to position 84 and position 85 of SEQ ID NO:45.

17. The composition of any one of items 1-16, wherein the chimeric CMV polypeptide comprises or consists of the amino acid sequence of SEQ ID NO:10, SEQ ID NO:11, or SEQ ID NO: 12.

18. The composition of any one of items 1-16, wherein the chimeric CMV polypeptide comprises, optionally consists of, (i) a first polypeptide comprising or consisting of the amino acid sequence of SEQ ID NO:5, SEQ ID NO:45, or SEQ ID NO:52 and (ii) a second polypeptide comprising, optionally consisting of, a stretch of consecutive negative amino acids, wherein said negative amino acids are independently selected from aspartic acid and/or glutamic acid, wherein said second polypeptide is inserted optionally between the amino acid residues of position 88 and position 89 of SEQ ID NO:5, between the amino acid residues of position 84 and position 85 of SEQ ID NO:45, or between the amino acid residues of position 86 and position 87 of SEQ ID NO:52.

19. The composition of any one of items 1-18, wherein the IL-1β-D145X mutein is a mammalian IL-1β-D145X mutein, optionally a non-human mammalian IL-1β-D145X mutein.

20. The composition of item 19, wherein the non-human mammalian IL-1β-D145X mutein is an equine IL-1β-D145X mutein, a bovine IL-1β-D145X mutein, an ovine IL-1β-D145X mutein, a caprine IL-1β-D145X mutein, or a porcine IL-1β-D145X mutein.

21. The composition of any one of items 1-20, wherein the IL-1β polypeptide is a mammalian IL-1β polypeptide, optionally a non-human mammalian IL-1β polypeptide.

22. The composition of item 21, wherein the non-human mammalian IL-1β polypeptide is an equine IL-1β polypeptide, a bovine IL-1β polypeptide, an ovine IL-1β polypeptide, a caprine IL-1β polypeptide, or a porcine IL-1β polypeptide.

23. The composition of any one of items 1-22, wherein the amino acid residue X is selected from the group consisting of lysine (K), tyrosine (Y), phenylalanine (F), asparagine (N), and arginine (R).

24. The composition of item 23, wherein the amino acid residue X is lysine (K).

25. The composition of any one of the preceding items, wherein at least one of the at least one chimeric CMV polypeptide and/or at least one of the at least one antigen is recombinant.

26. The composition of any one of the preceding items, wherein the at least one chimeric CMV polypeptide is capable of forming a virus-like particle of CMV upon expression by self-assembly in E. coli.

27. The composition of any one of the preceding items, wherein the IL-1β-D145X mutein of the composition exhibits reduced and/or eliminated natural biological activity as compared to a counterpart wild type IL-1β polypeptide.

28. The composition of item 27, wherein the natural biological activity is measured in vivo.

29. The composition of item 27, wherein the natural biological activity is measured in vitro.

30. The composition of any one of the preceding items, wherein the composition exhibits greater antigenicity as compared to a wild type IL-1β polypeptide.

31. The composition of item 30, wherein the antigenicity is measured in vivo.

32. The composition of item 30, wherein the antigenicity is measured in vitro.

33. The composition of any one of the preceding items, wherein the composition is capable of inducing production anti-wild type IL-1β antibodies in an animal, when the composition is administered to the animal.

34. The composition of item 33, wherein the anti-wild type IL-1β antibodies are capable of neutralizing one or more natural biological activity of IL-1 β, when measured in an in vitro assay and/or in an in vivo assay.

35. The composition of any one of the items 33-34, wherein the IL-1β-D145X mutein of the composition is derived from the same species as the animal to which the composition is administered.

36. The composition of any one of the preceding items, wherein the administration of the composition to an animal (i) produces no adverse effects, at most mild adverse effects, or at most moderate adverse effects, (ii) produces no severe adverse effects, (iii) produces only adverse effects of short duration and/or of moderate duration, (iv) produces only localized adverse effects, (v) produces no systemic adverse effects, at most mild systemic adverse effects, or at most moderate systemic adverse effects, (vi) produces severe adverse effects of only short duration and/or of only moderate duration, and/or (vii) produces no severe systemic effects.

37. A composition, preferably a veterinary composition, comprising
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:45; and
   (ii) a polypeptide comprising, preferably consisting of, a stretch of consecutive negative amino acids, wherein said negative amino acids are independently selected from aspartic acid or glutamic acid, wherein said polypeptide is inserted between any amino acid residue of said CMV polypeptide corresponding to any amino acid residue between position 75 and position 85 of SEQ ID NO:45.
(b) at least one antigen, wherein said antigen comprises at least one second attachment site, and wherein said antigen is an interleukin 1β D145X mutein (IL-1 β-D145X mutein) antigen; and
wherein (a) and (b) are linked through said at least one first and said at least one second attachment site, preferably via at least one covalent non-peptide bond.

38. A composition, preferably a veterinary composition, comprising
(c) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:45; and
   (ii) a D/E polypeptide comprising or consisting of, a stretch of consecutive negative amino acids, wherein said negative amino acids are independently selected from aspartic acid and/or glutamic acid, wherein said polypeptide is inserted between any two adjacent amino acid residue of said CMV polypeptide corresponding to any two adjacent amino acid residue between position 75 and position 85 of SEQ ID NO:45.
(d) at least one antigen, wherein said antigen comprises at least one second attachment site, and wherein said antigen is an interleukin 1β D145X mutein (IL-1 β-D145X mutein) antigen; and
wherein (a) and (b) are linked through said at least one first and said at least one second attachment site, preferably via at least one covalent non-peptide bond.

39. The composition of item 37 or item 38, wherein said chimeric CMV polypeptide further comprises a T helper cell epitope.

40. The composition of item 39, wherein said T helper cell epitope replaces a N-terminal region of said CMV polypeptide, optionally wherein said replaced N-terminal region of said CMV polypeptide consists of 5 to 15 consecutive amino acids, 9 to 14 consecutive amino acids, 9 to 13 consecutive amino acids, 10 to 13 consecutive amino acids, 11 to 13 consecutive amino acids, 11 consecutive amino acids, 12 consecutive amino acids, or 13 consecutive amino acids.

41. The composition of item 40, wherein, said N-terminal region of said CMV polypeptide corresponds to amino acids 2-12 of SEQ ID NO:45.

42. The composition of item 41, wherein a methionine residue is inserted at the N-terminus of the T helper cell epitope.

43. The composition of any one of the items 39-42, wherein one, two, or three additional amino acids are inserted between the N-terminal methionine and the T helper cell epitope.

44. The composition of any one of the items 39-43, wherein said T helper cell epitope is derived from tetanus toxin, comprises or consists of a PADRE sequence, comprises or consists of the amino acid sequence of SEQ ID NO:47, or comprises or consists of the amino acid sequence of SEQ ID NO:48.

45. The composition of any one of the items 39-43, wherein said T helper cell epitope is derived from tetanus toxin or is a PADRE sequence, and wherein preferably said Th cell epitope comprises the amino acid sequence of SEQ ID NO:47 or SEQ ID NO:48.

46. The composition of CMV of any one of the items 37-45, wherein said CMV polypeptide (a) comprises SEQ ID NO: 45 or (b) comprises an amino acid sequence having sequence identity of at least 80%, at least 85%, at least 90%, at least 91%, at least 92%, at least 93%, at least 94%, at least 95%, at least at least 96%, or at least 97%, at least about 98%, or at least 99% to SEQ ID NO:45.

47. The composition of CMV of any one of the items 1-46, wherein said CMV polypeptide is a coat protein of CMV or an amino acid sequence having a sequence identity of at least 90%, preferably 95% with SEQ ID NO:45.

48. The composition of any one of the items 1-47, wherein said CMV polypeptide comprises, preferably consists of, the amino acid sequence of SEQ ID NO:5, wherein said polypeptide comprising said stretch of consecutive negative amino acids is inserted between amino acid residues of position 88 and position 89 of said SEQ ID NO:5.

49. The composition of CMV of any one of the items 1-48, wherein said stretch of consecutive negative amino acids has a length of 3 to 10 amino acids.

50. The composition of CMV of any one of the items 1-49, wherein said stretch of consecutive negative amino acids consists solely of glutamic acids.

51. The composition of CMV of any one of the items 37-50, wherein said stretch of consecutive negative amino acids comprises or consists of SEQ ID NO:1 or SEQ ID NO:2.

49. The composition of CMV of any one of the items 37-48, wherein said polypeptide comprising said stretch of consecutive negative amino acids further comprises (i) a first amino acid linker and/or (ii) a second amino acid linker; wherein, if present, said first amino acid linker is positioned at the N-terminus of the stretch of consecutive negative amino acids, and, if present, the second amino acid linker is positioned at the C-terminus of said stretch of consecutive negative amino acids.

50. The composition of CMV of any one of the items 1-49, wherein said polypeptide comprising said stretch of consecutive negative amino acids further comprises a first amino acid linker and a second amino acid linker, wherein said first amino acid linker is positioned at the N-terminus of said stretch of consecutive negative amino acids, and said second amino acid linker is positioned at the C-terminus of said stretch of consecutive negative amino acids, and wherein said first and said second amino acid linker is independently selected from the group consisting of:
(a.) a polyglycine linker (G-linker) having an amino acid sequence (Gly) n of a length of n=2-10;
(b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and
(c.) an amino acid linker (GS*-linker) comprising at least one Gly, at least one Ser, and at least one amino acid selected from Thr, Ala, Lys, and Cys.

51. The composition of CMV of any one of the items 37-50, wherein said first amino acid linker comprises, optionally consists of, SEQ ID NO:8 and/or said second amino acid linker comprises, optionally consists of, SEQ ID NO:4 or SEQ ID NO:9.

52. The composition of any one of the items 37-51, wherein said polypeptide comprises, preferably consists of, SEQ ID NO:49, SEQ ID NO:50 or SEQ ID NO:51.

53. The composition of any one of items 37-52, wherein said polypeptide comprising said stretch of consecutive negative amino acids is inserted between the two adjacent amino acid residues of said CMV polypeptide that correspond to position 84 and position 85 of SEQ ID NO: 45.

54. The composition of any one of items 37-53, wherein the chimeric CMV polypeptide comprises, optionally consists of, (i) a CMV polypeptide comprising or consisting of the amino acid sequence of SEQ ID NO:5, SEQ ID NO:45, or SEQ ID NO:52 and (ii) a polypeptide comprising, optionally consisting of, a stretch of consecutive negative amino acids, wherein said negative amino acids are independently selected from aspartic acid and/or glutamic acid, wherein said polypeptide is inserted optionally between the amino acid residues of position 88 and position 89 of SEQ ID NO:5, between the amino acid residues of position 84 and position 85 of SEQ ID NO:45, or between the amino acid residues of position 86 and position 87 of SEQ ID NO:52.

55. The composition of item 37 or item 38, wherein said chimeric CMV polypeptide comprises, preferably consists of, the amino acid sequence of SEQ ID NO:10, SEQ ID NO: 11 or SEQ ID NO:12.

56. The composition of any one of the items 1-55, wherein said at least one first attachment site is not comprised or part of the polypeptide comprising said stretch of consecutive negative amino acid.

57. The composition of any one of the items 1-56, wherein said first attachment site is an amino group, preferably an amino group of a lysine residue, and wherein said at least one second attachment site is a sulfhydryl group, preferably a sulfhydryl group of a cysteine residue.

58. The composition of any one of the items 37-57, wherein (a) and (b) are linked via a heterobifunctional cross-linker through said at least one first and said at least one second attachment site via at least one covalent non-peptide bond, optionally wherein said hetero-bifunctional cross-linker is SMPH, and wherein said at least one first attachment site is not comprised or is not part of the polypeptide comprising said stretch of consecutive negative amino acids, and wherein said at least one first attachment site is an amino group, optionally an amino group of a lysine, and wherein the at least one second attachment site is a sulfhydryl group, optionally a sulfhydryl group of a cysteine residue or a sulfhydryl group that has been chemically attached to the antigen.

59. The composition of any one of the preceding items, wherein said antigen is selected from an interleukin 1β D145X mutein (IL-13-D145X mutein) antigen, wherein the amino acid residue X is selected from the group consisting of lysine (K), tyrosine (Y), phenylalanine (F), asparagine (N), and arginine (R), wherein preferably the amino acid residue X is lysine (K).

60. The composition of any one of the preceding items, wherein said antigen is selected from an interleukin 1β D145K mutein (IL-1β-D145K mutein) antigen.

61. The composition of any one of the items 37-60, wherein at least one of the at least one chimeric CMV polypeptide and/or at least one of the at least one antigen is recombinant.

62. The composition of any one of the items 37-61, wherein the at least one chimeric CMV polypeptide is capable of forming a virus-like particle of CMV upon expression by self-assembly in E. coli.

63. The composition of any one of the items 37-62, wherein the IL-1β-D145X mutein of the composition exhibits reduced and/or eliminated natural biological activity as compared to a counterpart wild type IL-1β polypeptide.

64. The composition of item 63, wherein the natural biological activity is measured in vivo.

65. The composition of item 63, wherein the natural biological activity is measured in vitro.

66. The composition of any one of the items 37-65, wherein the composition exhibits greater antigenicity as compared to a wild type IL-1β polypeptide.

67. The composition of item 66, wherein the antigenicity is measured in vivo.

68. The composition of item 66, wherein the antigenicity is measured in vitro.

69. The composition of any one of the items 37-68, wherein the composition is capable of inducing production anti-wild type IL-1β antibodies in an animal, when the composition is administered to the animal.

70. The composition of item 69, wherein the anti-wild type IL-1β antibodies are capable of neutralizing one or more natural biological activity of IL-1β, when measured in an in vitro assay and/or in an in vivo assay.

71. The composition of any one of the items 37-70, wherein the administration of the composition to an animal (i) produces no adverse effects, at most mild adverse effects, or at most moderate adverse effects, (ii) produces no severe adverse effects, (iii) produces only adverse effects of short duration and/or of moderate duration, (iv) produces only localized adverse effects, (v) produces no systemic adverse effects, at most mild systemic adverse effects, or at most moderate systemic adverse effects, (vi) produces severe adverse effects of only short duration and/or of only moderate duration, and/or (vii) produces no severe systemic effects.

72. A method of inducing neutralizing antibodies against IL-1β in an animal wherein said method comprising administering a composition of any one of the items 1 to 71.

73. The composition of any one of the items 1-71 for use in a method of inducing neutralizing antibodies against IL-1β in an animal.

### EXAMPLES

### Example 1

### Construction and Production of Surface Charge Modified CMV VLPs

Different chimeric CMV polypeptides in accordance with the present invention were prepared, and subsequently expressed leading to the inventive modified CMV VLPs.

Towards this end, chimeric CMV polypeptides comprising, in particular, different polypeptides of contiguous negative amino acids, namely polypeptides consisting of either 4, 8, or 12 glutamic acid residues (“E4”-SEQ ID NO:1; “E8”-SEQ ID NO:2; “E12”-SEQ ID NO:3) were prepared such that said glutamic acid residues were inserted between amino acid residues Ser (88) and Tyr (89) of the M-CMV polypeptide CMV-Ntt830 (SEQ ID NO:5). Said M-CMV polypeptide CMV-Ntt830 comprises the T helper cell epitope derived from tetanus toxoid TT830 (SEQ ID NO:6). The corresponding nucleic acid sequence (SEQ ID NO:7) coding for said M-CMV polypeptide CMV-Ntt830 was prepared as described in Example 3 of WO2016/062720A1.

The prepared chimeric CMV polypeptides further comprise linkers flanking the introduced E4, E8 and E12 polypeptides at both termini. In detail, said prepared chimeric CMV polypeptides either comprise a GGS-linker or a GGGS-linker (SEQ ID NO:8) directly at the N-terminus of the introduced E4, E8, and E12 polypeptides, and either a GGGSGS-linker (SEQ ID NO:9) or a CGGGSGS-linker (SEQ ID NO:4) directly at the C-terminus of the introduced E4, E8, and E12 polypeptides.

The resulting amino acid sequences of said prepared chimeric CMV polypeptides are named “CMV-Ntt830-E4”, “CMV-Ntt830-E8”, “CMV-Ntt830-E8*” and “CMV-Ntt830-E12” and have the amino acid sequences as follows:
“CMV-Ntt830-E4”: SEQ ID NO:10;
“CMV-Ntt830-E8”: SEQ ID NO:11;
“CMV-Ntt830-E8*”: SEQ ID NO:12;
“CMV-Ntt830-E12”: SEQ ID NO:13.

The corresponding nucleotide sequences of said preferred chimeric CMV polypeptides are as follows:
“CMV-Ntt830-E4”: SEQ ID NO:14;
“CMV-Ntt830-E8”: SEQ ID NO:15;
“CMV-Ntt830-E8*”: SEQ ID NO:16;
“CMV-Ntt830-E12”: SEQ ID NO:17.

First, the chimeric CMV polypeptide CMV-Ntt830-E8* was prepared. Hereby and in a first step the incorporation of the coding sequence for E8 including the flanking linkers into the modified CMV using PCR mutagenesis was effected. The PCR fragment coding for the E8 sequence including the flanking linkers as well as the 3′ end fragment of the modified CMV was amplified in two step PCR using the following oligonucleotides:
Forward: E8*-1F (SEQ ID NO:18)
Forward: E8*-2F (SEQ ID NO:19)
Reverse: CMcpR (SEQ ID NO:20).

Thus, a PCR reaction was carried out using E8*-1F/CMcpR oligonucleotides and pET-CMV-Ntt830 plasmid as template. The template pET-CMV-Ntt830 was prepared as described in Example 3 of WO2016/062720A1 (and corresponding Example of U.S. Pat. No. 10,532,107). The target PCR product was obtained after a second PCR using oligonucleotides E8*-2F/CMcpR and the PCR product from the first PCR. The resulting PCR product was cloned into helper vector pTZ57 (InsTAclone PCR Cloning Kit, Fermentas #K1214). PCR product-containing plasmid was amplified in E. coli XL1-Blue cells, and plasmid DNA was purified and sequenced using BigDye cycle-sequencing kit and an ABI Prism 3100 Genetic Analyzer (Applied Biosystems). As a result, the helper plasmid pTZ-CMV-E8*, without PCR errors, was obtained.

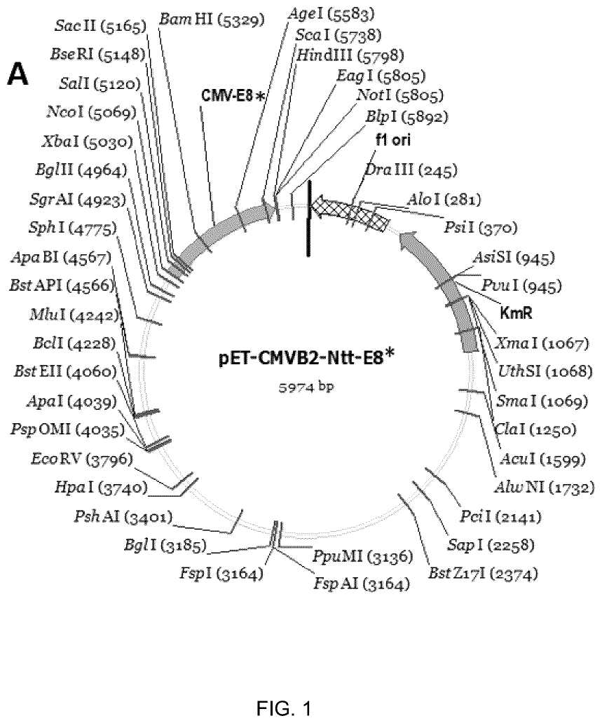

As a next step, the BamHI/HindIII fragment of pTZ-CMV-E8* was cloned back into the pET-CMV-Ntt830B helper vector using the same restriction sites, resulting in the expression vector pET-CMVB2-Ntt-E8C (FIG. 1).

The helper vector pET-CMV-Ntt830B was used for introduction of polypeptides comprising a stretch of consecutive negative amino acids coding DNA sequences in the corresponding CMV DNA sequence of CMV-Ntt830, BamHI site-containing sequence was introduced at the corresponding position for subsequent cloning. The CMV-Ntt830 coding nucleic acid sequence was prepared as described in Example 3 of WO2016/062720A1 and corresponds to SEQ ID NO:14 of WO2016/062720A1.

The BamHI site was introduced by two-step PCR mutagenesis using below listed oligonucleotides and previously constructed pET-CMV-Ntt830 as a template. As indicated, the template pET-CMV-Ntt830 was prepared as described in Example 3 of WO2016/062720A1.
1^{st} PCR: Forward-pET-90 primer (anneals pET28a+) (SEQ ID NO:21)
   Reverse-RGSYrev (SEQ ID NO:22)
2nd PCR Forward-RGSYdir (SEQ ID NO:23)
   Reverse-CMV-AgeR (SEQ ID NO:24)

After purification of both PCR products, the next PCR was carried out to join the PCR fragments (5 cycles without primers then 25 cycles using primers pET-90 and CMV-AgeR).

After amplification of the gene, the obtained PCR product was directly cloned into the pTZ57R/T vector (InsTAclone PCR Cloning Kit, Fermentas #K1214). E. coli XL1-Blue cells were used as a host for cloning and plasmid amplification.

To avoid RT-PCR errors, several CMV-Ntt830 gene-containing pTZ57 plasmid clones were sequenced using a BigDye cycle sequencing kit and an ABI Prism 3100 Genetic analyzer (Applied Biosystems). After sequencing, pTZ-plasmid clone without sequence errors containing CMV-Ntt830B gene with introduced BamHI site was cut with NcoI and AgeI enzymes. Then the fragment was subcloned into the NcoI/AgeI sites of the pET-CMV-Ntt830, resulting in the helper vector pET-CMV-Ntt830B.

CMV-Ntt830-E8* VLPs were produced in E. coli C2566 cells (New England Biolabs, USA). The VLPs were produced using, E. coli cell cultivation, biomass treatment and purification methods as follows:
1) suspend 3 g biomass in 20 ml of 50 mM Na citrate, 5 mM Na borate, 5 mM EDTA, 5 mM mercaptoethanol, pH 9.0, treat the suspension with ultrasound (Hielscher sonicator UP200S, 16 min, amplitude 70%, cycle 0.5);
2) Centrifuge the lysate at 11000 rpm for 20 min, at +4° C.;
3) Prepare sucrose gradient (20-60%) in 35 ml tubes, in buffer containing 50 mM Na citrate, 5 mM Na borate, 2 mM EDTA, 0.5% TX-100;
4) Overlay 5 ml of the VLP sample over the sucrose gradient;
5) Centrifuge 6 h using SW32 rotor, Beckman (25000 rpm, at +18° C.).
6) Divide the content of each gradient tube in 6 ml fractions. Pool corresponding fractions;
7) Analyse gradient fractions on SDS.

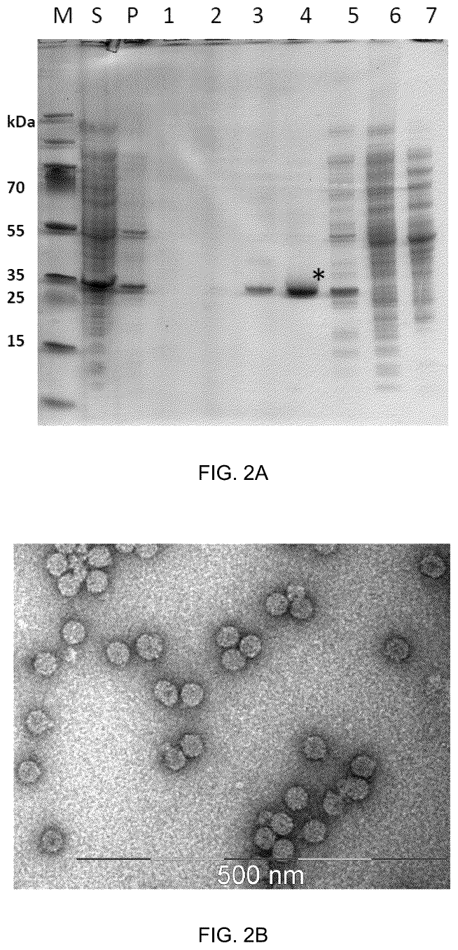

SDS-PAGE analysis of the VLPs after sucrose gradient purification demonstrates homogeneous CMV-Ntt830-E8* coat protein monomer (FIG. 2A) and electron microscopy shows intact VLPs (FIG. 2B).

The chimeric CMV polypeptides CMV-Ntt830-E4, CMV-Ntt830-E8 and CMV-Ntt830-E12 were prepared accordingly and as follows. The first step was the incorporation of the poly-glutamate coding sequences including the flanking linkers into the modified CMV using PCR mutagenesis. The PCR fragments coding for poly-glutamate sequences including the flanking linkers as well as the 3′ end fragment of the modified CMV were amplified by PCR using the following pairs of oligonucleotides and plasmid pET-CMVB2-Ntt-E8* as a template:
1) Forward: E4-F (SEQ ID NO:25)
   Reverse: CMcpR (SEQ ID NO:20);
2) Forward: E8-F (SEQ ID NO:26)
   Reverse: CMcpR (SEQ ID NO:20);
3) Forward: E12-F (SEQ ID NO:27)
   Reverse: CMcpR (SEQ ID NO:20).

The resulting PCR products were cloned into helper vector pTZ57 (InsTAclone PCR Cloning Kit, Fermentas #K1214). PCR product containing plasmids were amplified in E. coli XL1-Blue cells, and plasmid DNAs purified and sequenced using BigDye cycle-sequencing kit and an ABI Prism 3100 Genetic Analyzer (Applied Biosystems). Thus the helper plasmids pTZ-CMV-E4, pTZ-CMV-E8 and pTZ-CMV-E12 without PCR errors were obtained.

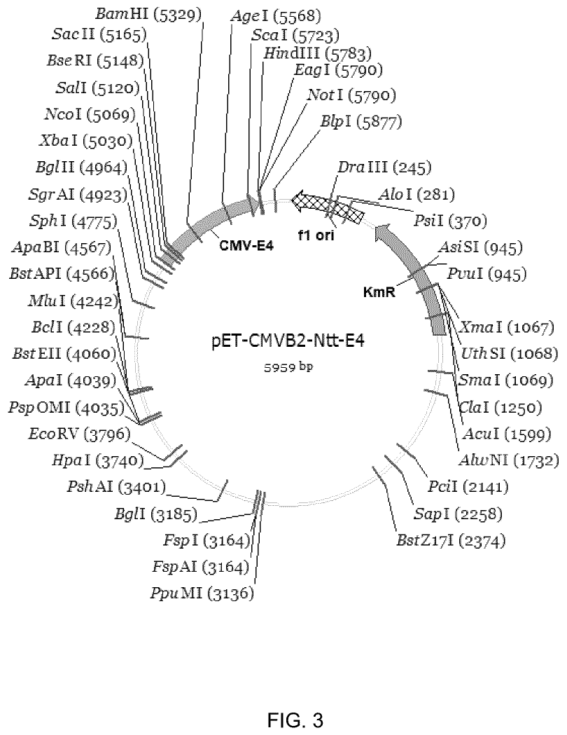

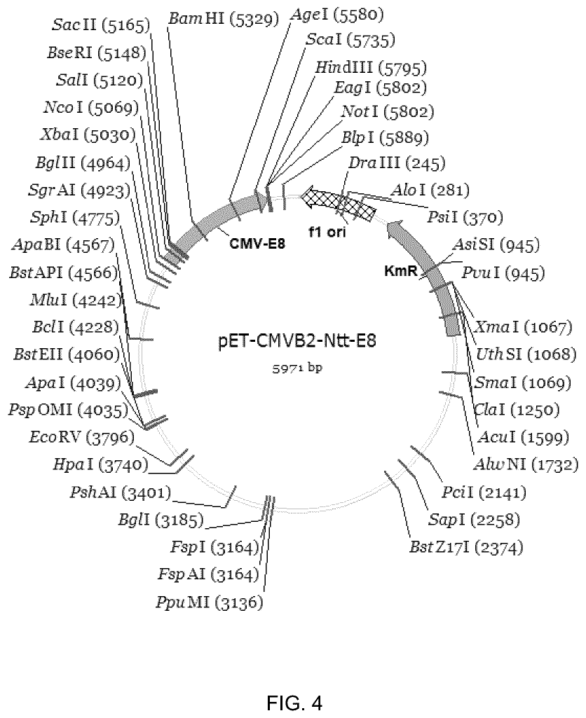

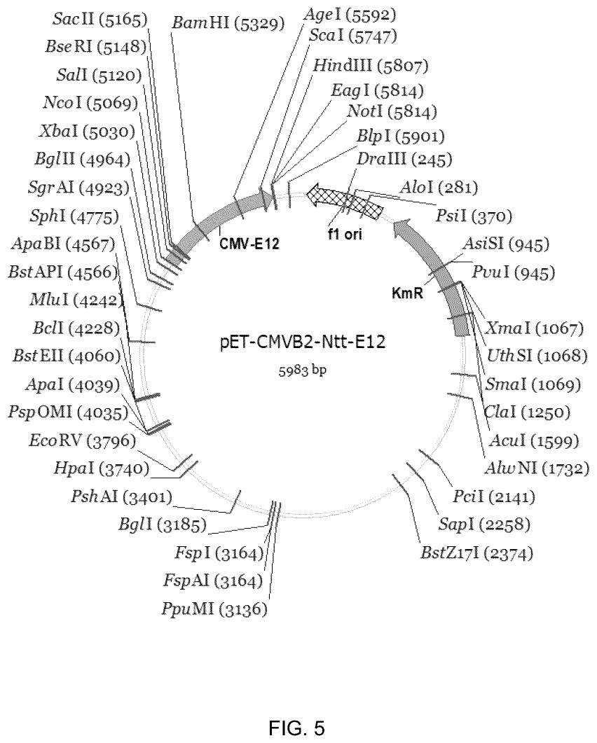

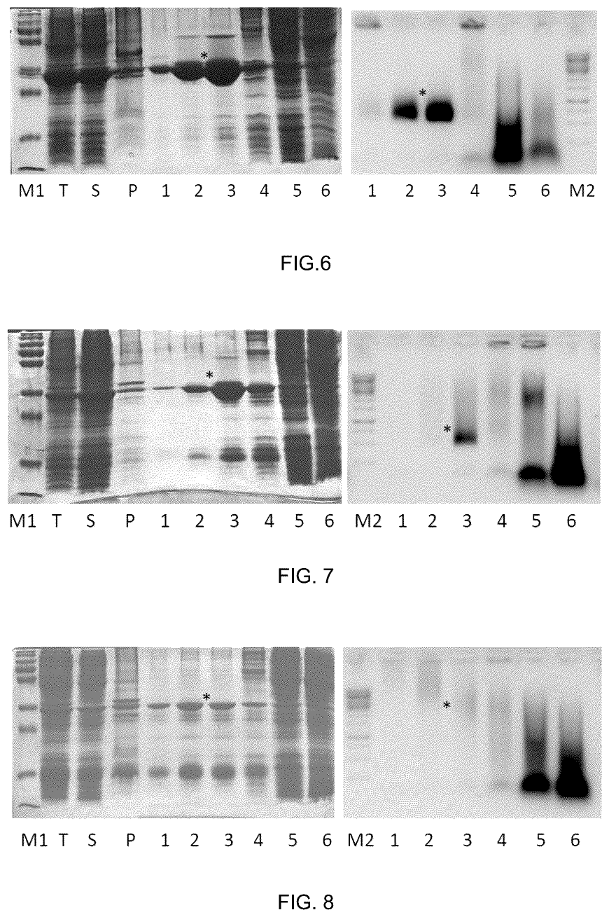

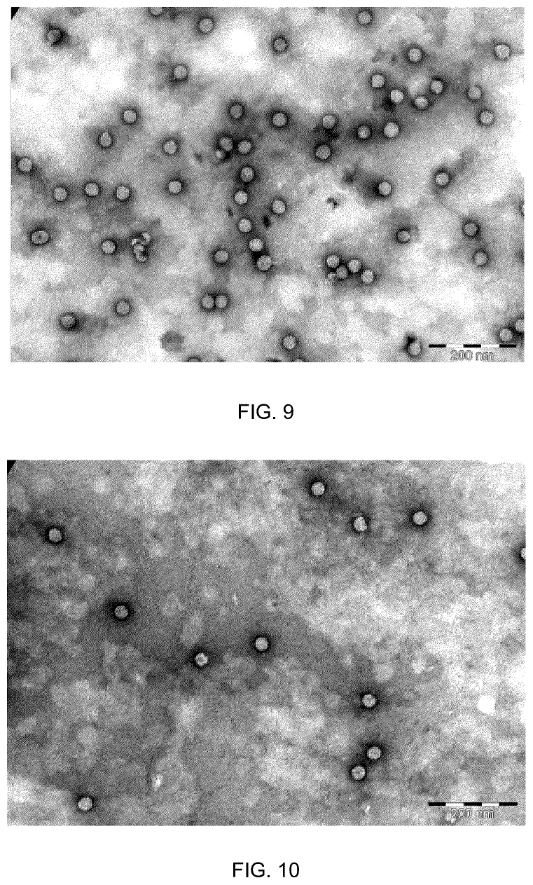

Next, the BamHI/HindIII digested fragments of pTZ-CMV-E4, pTZ-CMV-E8 and pTZ-CMV-E12 were cloned back into the pET-CMV-Ntt830B (see above) using the same restriction sites. The expression vectors pET-CMVB2-Ntt-E4 (FIG. 3) pET-CMVB2-Ntt-E8 (FIG. 4), and pET-CMVB2-Ntt-E12 (FIG. 5) were thus obtained. The expression vectors were transformed into E. coli C2566 cells (New England Biolabs, USA). VLPs were produced using, E. coli cell-cultivation, biomass-treatment and purification methods as described above for CMV-Ntt830-E8* VLPs. SDS-PAGE analyses of the VLPs after sucrose gradient purification demonstrated near homogeneous CMV-coat protein monomer was obtained for all 3 poly-glutamate constructs (FIG. 6, FIG. 7, FIG. 8). However, agarose gel analysis showed integral particles were only formed with CMV-Ntt830-E4 and CMV-Ntt830-E8 but not with the CMV-E12 (FIG. 6, FIG. 7, FIG. 8). Electron microscopy showed that CMV-Ntt830-E4 and CMV-Ntt830-E8 formed intact VLPs (FIG. 9, FIG. 10).

### Example 2

### Stability of the Surface Charge Modified CMV VLPs as Compared to Non-Surface Charged CMV VLPs

### Thermal Stability

Increased thermal stability of the inventive surface charge modified CMV VLPs was demonstrated by measuring denaturation of the prior art CMV-Ntt830 VLPs, which were prepared as described in Example 3 and Example 4 of WO2016/062720A1, and of the inventive CMV-Ntt830-E4 VLPs as a function of increasing temperature and determining the respective melting points.

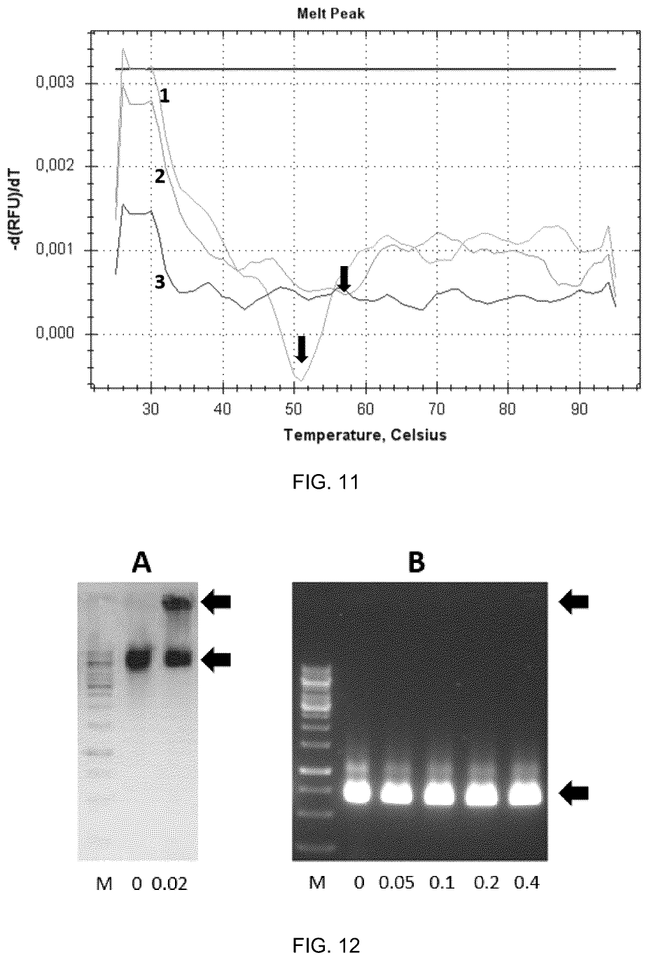

A thermal shift assay involving temperature-induced denaturation and the fluorescent dye SYPRO® Orange (Sigma, Saint Louis, USA) was used for this purpose. The dye is a naturally quenched in solution but as the VLPs denature with increasing temperatures, SYPRO® Orange interacts with exposed hydrophobic amino acids and cores and emits a fluorescent signal, which is measured by fluorometry. From the resultant melting curve (fluorescent signal vs temperature), the melt peak curves and melting temperature were determined. Solutions containing 0.5 mg/ml of sucrose density gradient purified (as described in Example 1 above) CMV-Ntt830 VLPs or CMV-Ntt830-E4 VLPs in 5 mM Na phosphate, 2 mM EDTA, pH 7.5 were assayed with a real-time PCR system MJ Mini (Bio-Rad, Hercules, USA) using a DNA melting point determination program. Data were analysed using Opticon Monitor Software and melting curves processed at a smooth setting of four. FIG. 11 shows the melt peak curves for purified CMV-Ntt830 VLPs and CMV-Ntt830-E4 VLPs.

The respective melting temperatures were estimated to be 51° C. and 57° C. evidencing an increased thermal stability of the surface charge modified CMV VLPs in accordance with the invention as compared to the prior art CMV-Ntt830 VLPs.

### Ionic Strength/Salt Stability

Ionic strength is important for capsid stability. Salts in solution interact with charged residues on the coat proteins and VLP surfaces, influence the water shell and disfavour hydrophobic exposure and thereby influence overall VLP stability.

The relative stabilities of CMV-Ntt830 VLPs and CMV-Ntt830-E4 VLPs to NaCl were tested by incubating purified VLPs (0.5 mg/ml in 5 mM Na phosphate, 2 mM EDTA, pH 7.5) at room temperature with various NaCl concentrations. After 2 hours in the presence of 20 mM NaCl, the CMV-Ntt830 VLPs were relatively unstable and formed aggregates in significant proportion that were both visible to the eye and demonstrable by native gel electrophoresis (FIG. 12). In contrast, there was no evidence of aggregate formation for CMV-Ntt830-E4 VLPs even with NaCl concentrations up to 0.4 M (FIG. 12).

The improved stability in higher salt solution arising from the surface charge modifications of the inventive modified CMV VLPs is important for its processability by ion-exchange chromatography as described in Example 3.

### Example 3

### Purification Potential of Surface Charge Modified CMV VLPs as Compared to Non-Surface-Charged CMV VLPs

The sucrose gradient/cushion ultra-centrifugation purification step, which was used in the laboratory-scale CMV VLP manufacture process as described in the prior art such as in Examples 2-4 of WO2016/062720A1 and for the preparation of the inventive modified CMV VLPs as described in Example 1 above, provides CMV VLPs of suitable yield and purity for subsequent conjugation, vaccine manufacture and preclinical evaluation. However, this method cannot be simply and cost effectively used to produce vaccine for commercial purposes.

Ion exchange chromatography (IEX) is typically readily scalable and used in downstream processes for the commercial production of biologics. It is based on reversible ionic interactions between charged molecules/macromolecules in solution and an immobilized oppositely charged chromatography resin. An example is anion-exchange chromatography (AEX) where the stationary phase (resin) is positively charged and negatively charged molecules such as proteins are bound. The interaction of the resin and sample can be disrupted by application of a counter ion such as Cl—. IEX is commonly used in bind/elute mode to provide rapid capture, high-resolution purification and concentration of the desired sample. It can be employed in the initial (e.g. after lysate clarification), intermediate or penultimate stages of a downstream process.

For CMV VLPs to be effectively bound and eluted by IEX, it is necessary that the CMV VLP is stable to the ionic environment encountered during the binding and elution phases. Both the charge on the ion-exchange resin and elution salt contribute to the ionic environment.

The prior art CMV-Ntt830 VLPs as well as the inventive modified CMV VLPs such as CMV-Ntt830-E4, CMV-Ntt830-E8 and CMV-Ntt830-E8* have a net negative charge at about pH's of 9 and below, as demonstrated by their migration towards the positively charged electrode in NAGE. Thus, anion-exchange chromatography (AIX) is a technique that would have been expected to work for both CMV VLP particles.

However, this is not the case because the CMV-Ntt830 VLPs, as described above in Example 2, are relatively unstable in solution in the presence of already 20 mM NaCl and form aggregates, which precipitate. In contrast, the inventive modified CMV VLPs such as the CMV-Ntt830-E4 VLP do not form aggregates at NaCl concentrations up to 0.4 M (FIG. 12, Panel B). The improved stability in higher salt solution arising from the surface charge modifications to the VLP is essential for its processability by ion-exchange chromatography.

### Purification by Anion Exchange Chromatography (AEX).

To test the processability of prior art CMV-Ntt830 VLPs with anion exchange chromatography (AEX), sucrose gradient purified VLPs were prepared as described in Examples 2-4 of WO2016/062720A1. Five mls of CMV-Ntt830 VLPs (1 mg/ml) were buffer exchanged into 5 mM sodium borate pH 9 and loaded onto a 1.0 ml Macro-Prep DEAE Bio-Rad anion exchange cartridge equilibrated with the same buffer. After the loading step, the concentration of NaCl in the elution buffer was increased in step-wise manner (0.1, 0.2, 0.3, 0.4. 0.5, 0.8, 1.0 and 2.0 M). Fractions were collected and measured at 260 nm using Nanodrop spectrophotometer to measure protein and subjected to native agarose gel electrophoresis (NAGE).

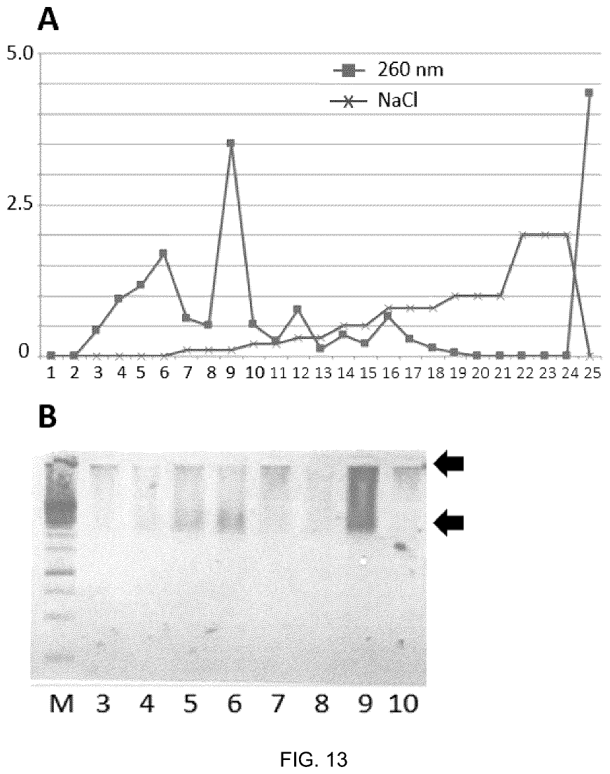

The resultant chromatogram of protein elution and NaCl concentrations plotted against the corresponding fraction (FIG. 13, panel A) shows the CMV-Ntt830 VLPs did not elute as a single peak as is typical for AIX. Instead, CMV-Ntt830 VLPs eluted in a broad non-specific manner during the loading (at 0 M NaCl) and subsequent elution steps over a range of NaCl concentrations, principally 0.2 to 0.8 M. Critically, the VLP-containing fractions after elution from the column were turbid and contained a significant proportion of aggregated VLPs, as demonstrated by the presence of ethidium bromide stained VLPs in the loading wells following NAGE (FIG. 13, panel B). The propensity of the CMV-Ntt830 VLPs to aggregate and elute in a non-discrete manner precludes the ready use of this methodology for scale-up manufacture.

In contrast, non-aggregated CMV-Ntt830-E4 VLPs could be readily purified from a crude lysate using AEX. Clarified lysate prepared from E. coli expressing CMV-Ntt830-E4 VLPs (as described in Example 1) in 50 mM citrate, 5 mM Borate buffer pH 9.0 was loaded onto 60 ml of Fracto-DEAE (Merck) in an XK 26/20 column equilibrated with the same buffer and eluted by applying a continuous NaCl gradient from 0 to 1.0 M in the same buffer. The eluate was monitored at A260 nm to measure protein and conductivity measured to monitor salt concentration. The clarified lysate, flow-through and fractions were collected and subjected to NAGE and SDS-PAGE.

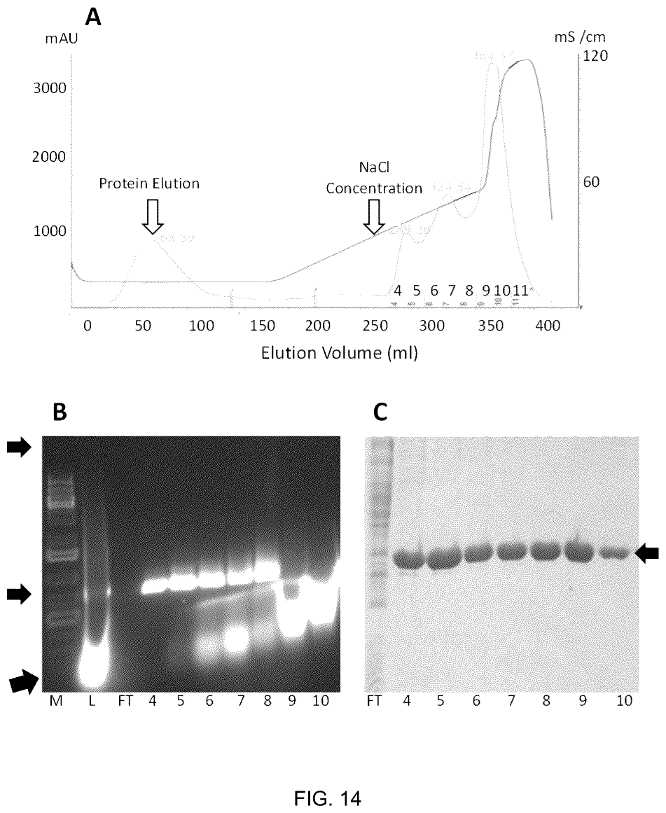

The resultant chromatogram, SDS-PAGE and NAGE analyses (FIG. 14) show that the CMV-Ntt830-E4 VLPs were not present in the flow-through and entirely bound to the Fracto-DEAE. The VLPs were subsequently eluted over a relatively narrow concentration range of 0.2-0.5M NaCl. Moreover, there was no evidence of aggregated VLPs in the loading wells of the native agarose gel. The Coomassie blue stained SDS-polyacrylamide gel showed highly pure VLP coat protein was obtained from the crude bacterial lysate.

### Example 4

### Cloning, Expression and Purification of Recombinant Canine IL-1β-D145K Mutein

### Cloning of Recombinant Canine IL-1β-D145K Mutein

The canine IL-1β-D145K mutein (SEQ ID NO:28) comprised in very preferred compositions of the present invention aimed to have a lower bioactivity as compared to its wild-type canine IL-1β (SEQ ID NO:29) (Ju et al., 1991, Proc. Nati. Acad. Sci., 88:2658-2662). The amino acid sequence of said canine IL-1β-D145K mutein (SEQ ID NO:28) is hereby mutated at its position 145, wherein said mutation is a replacement (as compared to the SEQ ID NO:29) of the amino acid aspartic acid (D) by the amino acid lysine (K).

The resulting amino acid sequence of the cIL-1β-D145K antigen used for coupling to modified CMV VLPs in accordance with the present invention is provided as SEQ ID NO:30. This amino acid sequence of SEQ ID NO:30 comprise the cIL-1β-D145K sequence of SEQ ID NO:28 to which a His6-tag (SEQ ID NO:31) and a four amino acid linker (SEQ ID NO:32) at the C-terminus are added. SEQ ID NO:30 further comprises amino acids derived from the start codon and the BamHI restriction site at the N-terminus as well derived from the transcription of the SpeI restriction site at the C-terminus. The corresponding nucleotide sequence of this cIL-1β-D145K antigen is described in SEQ ID NO: 33.

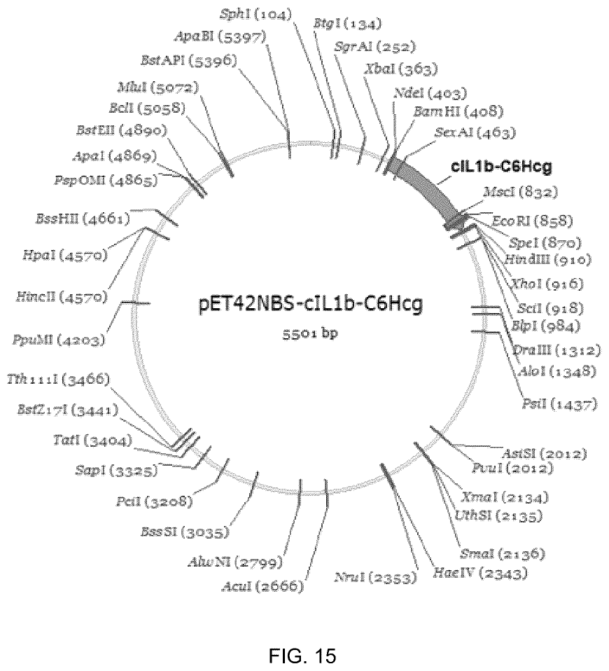

For producing the canine IL-1β-D145K antigen of SEQ ID NO:30, the aspartic acid amino acid at position 145 of the corresponding mature canine IL-1β (SEQ ID NO:29) was replaced with a lysine by PCR mutagenesis using the pET42NBS-cIL1b-C6Hcg plasmid as the template DNA and the primers cIL1b-BamF (SEQ ID NO:34) and cIL1b-145K-SpeR: (SEQ ID NO:35). The map of the expression vector pET42NBS-cIL1b-C6Hcg is shown in FIG. 15. For said template plasmid, the underlying and corresponding canine IL-1β sequence with flanking BamHI/SpeI restriction sites and without a “stop”-codon was produced by oligonucleotide directed gene synthesis (BioCat GmbH). After synthesis of the gene, it was excised from a pUC57 helper plasmid and sub-cloned into the Bam HI/Spe I sites of a modified pET42 vector.

The resultant product of the above indicated PCR mutagenesis was cloned into the cloning vector pTZ57R (Fermentas) and the plasmid then transformed into E. coli XL-1-blue (Agilent). The plasmid from a single colony was purified and the cIL1b-D145K-C6Hcg gene confirmed to have the correct nucleotide sequence subcloned into the pET42NBS-C6Hcg vector, a derivative of the pET42 (+) plasmid containing additional BamHI and SpeI cloning sites and the gene sequence coding for SEQ ID NO:36, resulting in the generation of the pET42NBS-cIL1b-D145K-C6Hcg plasmid. That the DNA sequence of the gene insert in the pET42NBS-cIL1b-145K-C6Hcg plasmid was correct, was confirmed by gene sequencing.

### Expression and Purification of Recombinant Canine IL-1β-D145K Mutein

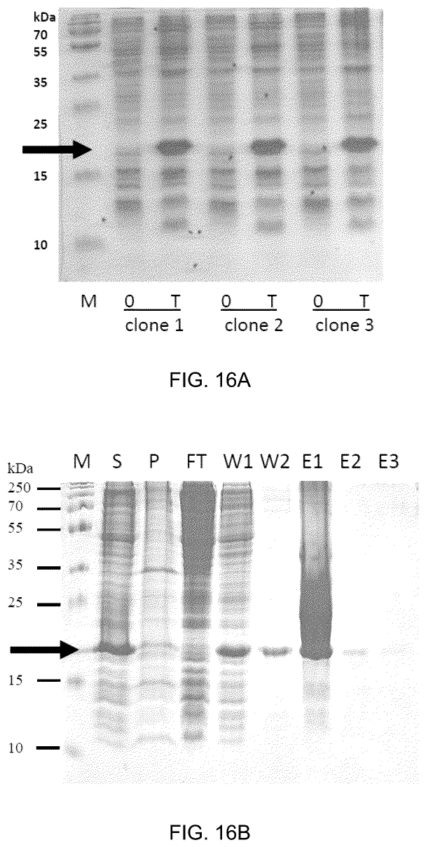

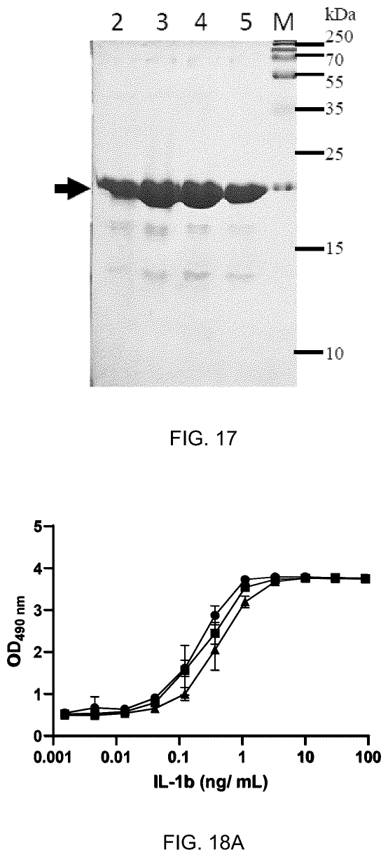

The expression vector pET42NBS-cIL1b-D145K-C6Hcg was transformed into E. coli C2566 cells (New England Biolabs, Ipswich, USA) resulting in the expression clones named C2566/pET42NBS-cIL1b-D145K-C6Hcg. Three clones were selected and expression of the canine IL-1β-D145K antigen of SEQ ID NO:30 was performed in the following way: Cultures of E. coli harboring the expression plasmid were grown in 100 mL 2×YT medium containing kanamycin (25 mg/L) and 0.1% (v/v) glucose on a rotary shaker (200 rev/min; Infers, Bottmingen, Switzerland) at 30° C. to an OD600 of 0.8-1.0. The expression of the canine IL-1β-D145K antigen of SEQ ID NO:30 was then induced by adding 0.2 mM IPTG. The medium was additionally supplemented with MgCl₂ to a final concentration of 5 mM. Incubation was continued on a rotary shaker at +20° C. for 18 hours (FIG. 16A). The resulting biomass was collected by low-speed centrifugation and frozen at −80° C. until purification. The canine IL-1β-D145K mutein antigen was purified using the Protino, Ni-IDA (Macherey-Nagel) single use immobilized metal ion affinity chromatography (IMAC) columns according to manufacturer's instructions. The biomass of a 100 mL culture was thawed and resuspended in 10 mL LEW buffer. Cells were lysed by sonication on ice for 16 minutes using an UP200S (Hielscher) ultrasound device run at an amplitude of 70% and a 0.5 second on/off pulsating mode. The lysate was centrifuged for 20 min at 11,000 rpm and +4° C. (sample S and P FIG. 16B). The clarified soluble fraction was applied to a Protino, Ni-IDA packed column 2000 (Macherey-Nagel), washed twice (sample W1 and W2, FIG. 16B) and eluted with 3×3 ml of imidazole containing elution buffer (sample E1, E2 and E3, FIG. 16B). Fractions containing the canine IL-1β-D145K antigen were identified by SDS-PAGE (FIG. 16B). The protein was further purified by gel filtration (Superdex 75; running buffer 5 mM Na phosphate, 2 mM EDTA, pH 7.5). Elution fractions containing the canine IL-1β-D145K were pooled and the protein in the sample concentrated using an Amicon-Ultra-15 (10 kDa) device. The protein concentration (Qubit, Invitrogen #Q33211) and UV absorbance (NanoDrop 1000) at 230 nm, 260 nm and 280 nm of the final protein sample were determined (not shown). The purity of the purified canine IL-1β-D145K was demonstrated by SDS-PAGE (FIG. 17) and the identity by a canine IL-1β-specific sandwich ELISA, an IL-1RI binding ELISA and a HEK cell-based IL-1β bioassay. The same assays were also used to measure the bioactivity of the cIL-1b-D145K-CMV vaccine.

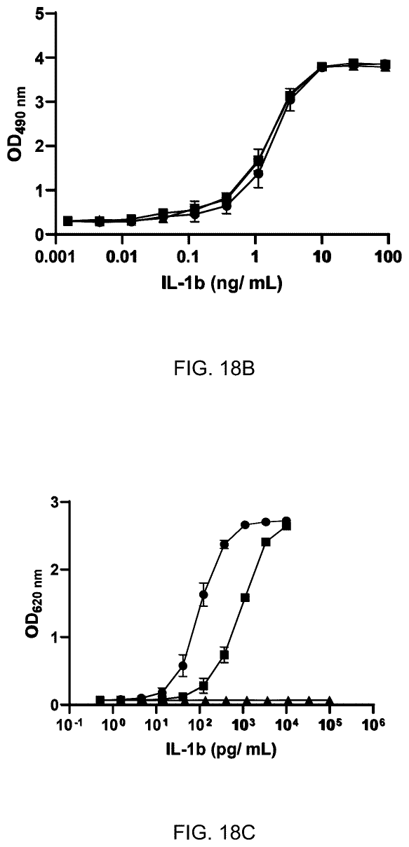

Briefly, for the canine IL-1β-specific ELISA the vaccines and cytokines were tested in a canine IL-1β-specific ELISA (R&D 3747-CL) to assess their ability to bind to canine IL-1β-specific antibodies. ELISA plates were coated overnight with 100 μl/well capture antibody at 800 ng/ml in PBS. After washing, plates were blocked with 1% BSA in PBS. Samples and standards were serial diluted, added to the ELISA plates for 2 hours at room temperature and then washed off. The biotinylated anti-cIL-1β detection antibody was added at a concentration of 200 ng/ml. Plates were incubated for 2 hours at room temperature and washed before adding the streptavidin-HRP reagent supplied by the kit for 20 minutes. A final washing step was performed and O-phenylenediamine dihydrochloride (OPD) reagent was added. The color reaction was stopped by addition of sulfuric acid and plates read at OD490 nm. Using GraphPad Prism and Microsoft Excel, sample concentrations were plotted against OD490 nm values. A 4-parameter logistic regression model was used for curve fitting and to calculate the EC50 values of each sample i.e. protein concentration resulting in half-maximal OD490 nm value. FIG. 18A shows the recombinant canine IL-1β-D145K mutein produced as described above was positive in a canine IL-1β-specific ELISA.

The binding of the vaccines and cytokines to the IL-1RI, the natural receptor of IL-1β, was tested to confirm that the mutein maintained the conformational structure of the wild type canine IL-1β. As canine IL-1RI was not commercially available, binding of the products to recombinant mouse IL-1RI was assessed instead. Briefly, ELISA plates were coated overnight with 100 μl/well recombinant mouse IL-1RI Fc chimera (R&D 771-MR-100) at 1 μg/ml in carbonate buffer (0.1M NaHCO₃ pH 9.6). After washing, plates were blocked with Superblock in PBS (Thermofisher 37515). Samples and standards were serial diluted, added to ELISA plates for at least 1 hour at room temperature before being washed off. The biotinylated anti-cIL-1β detection antibody (R&D DY3747) was then added at a concentration of 200 ng/ml. Plates were incubated for 1 hour at room temperature and washed before adding the streptavidin-HRP (JacksonImmunoResearch 016-030-084 diluted 1:1000) for 1 hour. A final washing step was performed and OPD reagent added. The color reaction was stopped with sulfuric acid and plates read at OD450 nm. Using GraphPad Prism and Microsoft Excel, sample concentrations were plotted against OD450 nm values. A 4-parameter logistic regression model was used for curve fitting and to calculate the EC50 values of each sample i.e. protein concentration resulting in half-maximal OD490 nm value. Comparable binding of the canine IL-1β-D145K mutein, of the corresponding canine IL-1β wild type of SEQ ID NO:29 (analogously prepared as described herein for IL-1β-D145K mutein; detailed synthesis described in PCT/EP2023/073756) and a commercially available recombinant canine IL-1β protein (R&D 3747-CL-025) to the IL-1RI was observed (FIG. 18B).

The bioactivities of the canine IL-1β-D145K mutein was assessed relative to said commercially available canine IL-1β standard (R&D 3747-CL-025) and said wild type canine IL-1β using the HEK-Blue IL-1beta reporter cells (InvivoGen hkb-il1bv2). Briefly, HEK-Blue™ IL-1b cells were grown in DMEM Growth medium containing (DMEM with high glucose, 10% HI-FBS, 1× GlutaMAX, 1× antibiotic (Pen/Strep (100 U/ml penicillin, 100 μg/ml streptomycin) or anti-anti (Gibco Antibiotic-Antimycotic, Thermo Fisher Scientific 15240062)) and 100 μg/mL Zeocin (InvivoGen ant-zn-1)). On the day of the assay, cells were detached using 5 mM EDTA in PBS solution, washed with PBS, and then filtered through a 70 μm cell strainer. Cells were resuspended in bioassay medium for a final concentration of 5×10⁴ cells per well of a 96 flat bottom well plate. Cytokine and vaccines were added in 3-fold dilution series at starting concentrations of 10 ng/ml and 5 μg/mL respectively. Control wells without cytokines were run on each assay plate. Plates were incubated at 5% CO₂, +37° C. for approximately 20 hours. After 20 hours incubation, cell viability was assessed by visual inspection. 40 μL/well of cell supernatant was added to 160 μL/well QUANTI-Blue Solution (Invivogen rep-qbs). The color development was monitored by measuring OD620 nm 5 hours later.

To determine the IL-1β bioactivity of tested samples, the sample concentrations were plotted against OD620 nm values. A 4 parameter logistic regression model was used for curve fitting and to calculate the EC50 values of each sample tested i.e. protein concentration resulting in half-maximal OD620 nm value.

The bioactivity of the mutein canine IL-1β-D145K was greatly decreased relative to a commercially available canine IL-1b standard (R&D 3747-CL-025) and purified wild type canine IL-1β; in fact, the bioactivity of the mutein was below the detection limit of the assay (FIG. 18C).

### Example 5

### Coupling of Recombinant Canine IL-1β-D145K Mutein to Modified CMV VLPs

For covalent coupling of recombinantly produced canine IL-1β-D145K mutein to modified CMV VLPs prepared as described above, in particular to CMV-Ntt830-E4 VLPs, recombinant canine IL-1β-D145K mutein was treated with Tris (2-carboxyethyl)phosphin (TCEP) at a 10-fold molar excess of TCEP. Meanwhile, purified CMV-Ntt830-E4 VLPs were diluted to 1.5 mg/ml and reacted with heterobifunctional chemical cross-linker succinimidyl-6-(b-maleimidopropionamide) hexanoate (SMPH) for 1 hour at room temperature. SMPH contains a NHS ester which reacts with the lysine on the surface of the VLP. The amount of SMPH added was 5× molar excess over one VLP coat protein monomer. Cross-linkers which did not react with the VLP were removed by centrifugation using an Amicon-Ultra-15, PLHK Ultracel-PL Membran, 100K centrifugal filter (Merck-Millipore, #UFC910096). The SMPH-derivatized VLPs were then washed 4 times with 5 mM Na2HPO4, 2 mM EDTA (pH 7.5).

Next, and briefly, the recombinant canine IL-1β-D145K mutein was added to the VLPs in an about 0.25:1 molar ratio, with respect to the respective chimeric CMV polypeptide monomer, to the previously SMPH derivatized surface charge modified CMV VLPs for 3 hours at RT while shaking. The engineered free cysteine of the canine IL-1β-D145K reacted with the maleimide of the cross-linker SMPH bound to the VLPs to form a stable covalent linkage. The sample was then centrifuged at 14,000 rpm for 5 minutes to remove precipitates and canine IL-1β-D145K proteins that were not associated with the VLPs were removed using Amicon-Ultra-0.5, 100K filtration units 100K (Merck-Millipore, UFC510096) and 5 times washing with 5 mM NaHPO4, 2 mM EDTA (pH 7.5).

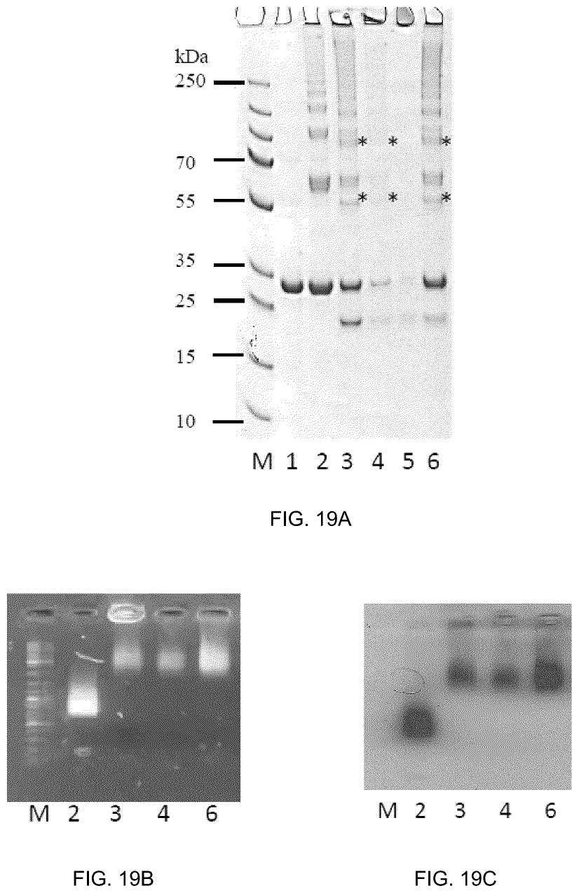

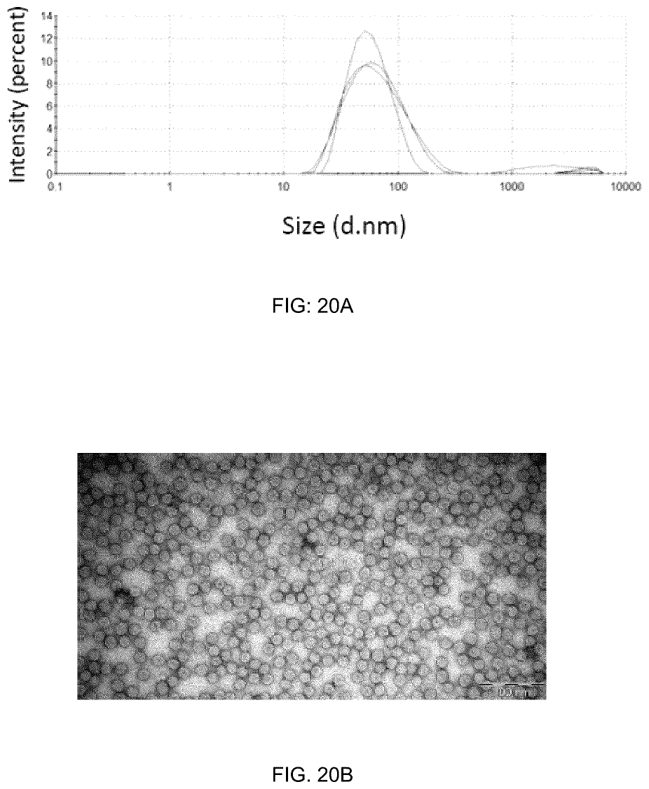

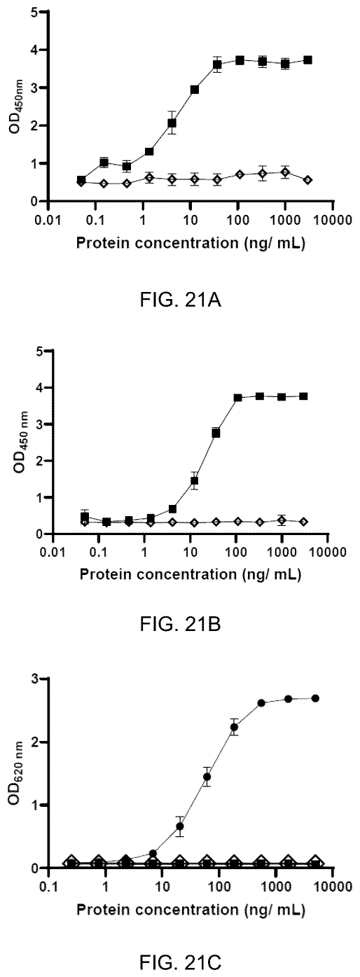

To demonstrate covalent conjugation of canine IL-1β-D145K antigen to VLPs, coupling reactions were analyzed by SDS-PAGE. Prominent conjugation bands were observed following chemical coupling of canine IL-1β-D145K with CMV-Ntt830-E4 VLPs (FIG. 19A). Analysis by native agarose gel electrophoresis showed co-localized bands stained for nucleic acid (FIG. 19B) and protein (FIG. 19C). This is indicative of integral RNA containing VLPs. The shift in migration of the cIL1b-D145K-CMVE4 VLPs relative to the CMV-Ntt830-E4 VLPs is due to the attachment of the positively charged canine IL-1β-D145K at neutral pH. Analysis by dynamic light scattering (DLS) and electron microscopy (FIG. 20A and FIG. 20B) showed the cIL1b-D145K-CMV-Ntt830-E4 VLP conjugates were of uniform size distribution, not aggregated and were stable in solution and formed typical VLP structures. Bioactivity testing of the cIL1b-D145K-CMV-Ntt830-E4 vaccine demonstrated that the VLPs are able to bind canine IL-1β-specific antibodies (FIG. 21A) and IL-1RI receptor (FIG. 21B) but do not activate the downstream signalling cascade of the IL-1β (FIG. 21C). This indicates that although the conformational structure of the IL-1β-D145K antigen incorporated into the VLPs is authentic, the substitution of the aspartic acid on position 145 with a lysine resulted in the loss of bioactivity of the cIL1b-D145K-CMV-Ntt830-E4 vaccine.

### Example 6

### Cloning, Expression and Purification of Recombinant Feline IL-1β-D145K Mutein

### Cloning of Recombinant Feline IL-1b-D145K Mutein

The gene coding for the wild type feline IL-1β protein without signaling sequence and neither a “start”-nor a “stop”-codon and with flanking BamHI/SpeI restriction sites was obtained by oligonucleotide directed gene synthesis in a pUC57 helper plasmid (BioCat GmbH, Heidelberg, Germany). The gene was then sub-cloned into the Bam HI/Spe I sites of a modified pET42 vector resulting in the production of the expression vector pET42NBS-fIL1b-c6Hcg. The resulting amino acid sequence of the so prepared feline IL-1β antigen is provided as SEQ ID NO:37. This amino acid sequence comprise the feline IL-1β sequence (SEQ ID NO:38) to which a His6-tag and a four amino acid linker (SEQ ID NO: 36) at the C-terminus are added. The corresponding nucleotide sequence of this feline IL-1b antigen is described in SEQ ID NO:39.

For the production of a feline IL-1β-D145K mutein (SEQ ID NO:40) with low bioactivity the aspartic acid amino acid at position 145 of the mature feline IL-1β of SEQ ID NO: 38 was replaced with a lysine. The resulting amino acid sequence of the so prepared fIL-1β-D145K antigen used for coupling to modified CMV VLPs in accordance with the present invention is provided as SEQ ID NO:41. This amino acid sequence comprises the fIL-1β-D145K sequence (SEQ ID NO:40) with an N terminal glycine and serine, a C terminal threonine and serine, to which a His6-tag and a four amino acid linker at the C-terminus are added. The corresponding nucleotide sequence of this fIL-1β-D145K antigen is described in SEQ ID NO:42.

For the production of the feline IL-1β-D145K antigen of SEQ ID NO:41, the aspartic acid amino acid at position 145 of the mature feline IL-1β of SEQ ID NO:38 was replaced with a lysine by PCR mutagenesis using the pET42NBS-fIL1b-C6Hcg plasmid as the template DNA and the primers fIL1b-o-BamF (SEQ ID NO:43) and f1IL1b-145K-SpeR (SEQ ID NO:44). The resultant PCR product was cloned into the cloning vector pTZ57R (Fermentas) and the plasmid then transformed into E. coli XL-1-Blue. The plasmid from a single colony was purified and the fIL1b-145K-C6Hcg gene confirmed to have the correct nucleotide sequence subcloned into the pET42NBS-c6Hcg vector, a derivative of the pET42 (+) plasmid containing additional BamHI and SpeI cloning sites and the gene sequence coding for SEQ ID NO:36, resulting in the generation of the pET42NBS-fIL1b-145K-C6Hcg plasmid.

### Expression and Purification of Recombinant Feline IL-1β-D145K Mutein

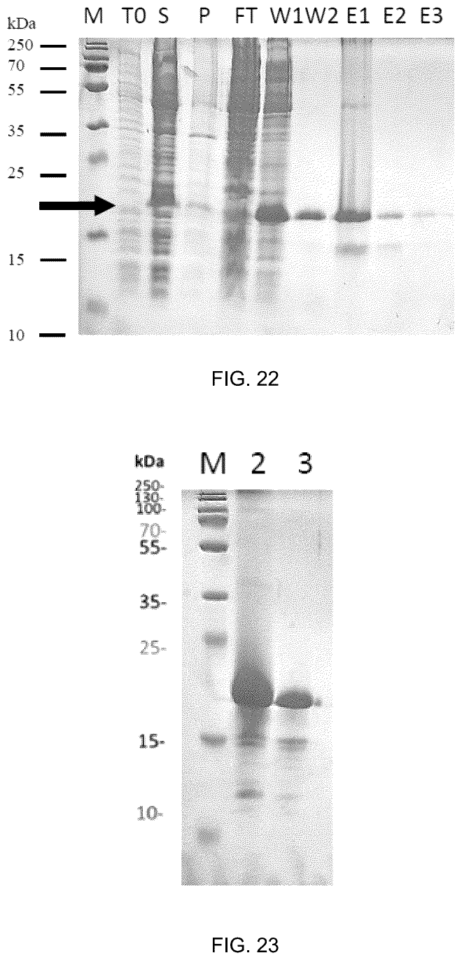

The expression vector pET42NBS-fIL1b-145K-C6Hcg was transformed into E. coli C2566 cells (New England Biolabs, Ipswich, USA) resulting in the expression clones named C2566/pET42NBS-fIL1b-145K-C6Hcg. Single colonies were picked and the expression of the feline IL-1β-D145K antigen of SEQ ID NO:41 was performed in the following way: Cultures of E. coli harboring the expression plasmid were grown in 200 mL 2× YT medium containing kanamycin (25 mg/L) and 0.1% (v/v) glucose on a rotary shaker (200 rev/min; Infers, Bottmingen, Switzerland) at 30° C. to an OD600 of 0.8-1.0. The expression of the feline IL-1β-D145K antigen of SEQ ID NO:41 was then induced by adding 0.2 mM IPTG (FIG. 22). The medium was additionally supplemented with MgCl₂ to a final concentration of 5 mM. Incubation was continued on a rotary shaker at +20° C. for 18 hours. The resulting biomass was collected by low-speed centrifugation and frozen at −80° C. until purification. The feline IL-1β-D145K mutein antigen was purified using the Protino, Ni-IDA (Macherey-Nagel) single use immobilized metal ion affinity chromatography (IMAC) columns according to manufacturer's instructions. The biomass of 200 mL culture was thawed and resuspended in 10 mL LEW buffer. Cells were lysed by sonication on ice for 16 minutes using an UP200S (Hielscher) ultrasound device run at an amplitude of 70% and a 0.5 second on/off pulsating mode. The lysate was centrifuged for 10 min at 14,000 rpm (sample S and P FIG. 22). The clarified soluble fraction was applied to a Protino, Ni-IDA packed columns 2000 (Macherey-Nagel), washed twice (sample W1 and W2 FIG. 22) and eluted with 3×3 ml of imidazole containing elution buffer (sample E1, E2 and E3 FIG. 22). Fractions containing the feline IL-1β-D145K antigen were identified by SDS-PAGE (FIG. 22). The protein was further purified by gel filtration (Superdex 75; running buffer 5 mM Na phosphate, 2 mM EDTA, pH 7.5). Elution fractions containing the feline IL-1β-D145K were pooled and the protein in the sample concentrated using an Amicon-Ultra-15 (10 kDa) device. The protein concentration (Qubit, Invitrogen #Q33211) and UV absorbance (NanoDrop 1000) at 230 nm, 260 nm and 280 nm of the final protein sample were determined (not shown). The purity of the purified feline IL-1β-D145K was demonstrated by SDS-PAGE (FIG. 23) and the identity by a feline IL-1β-specific sandwich ELISA, an IL-1RI binding ELISA and a HEK cell-based IL-1β bioassay. The same assays were also used to measure the bioactivity of the fIL-1b-D145K-CMV-Ntt830-E4 vaccine.

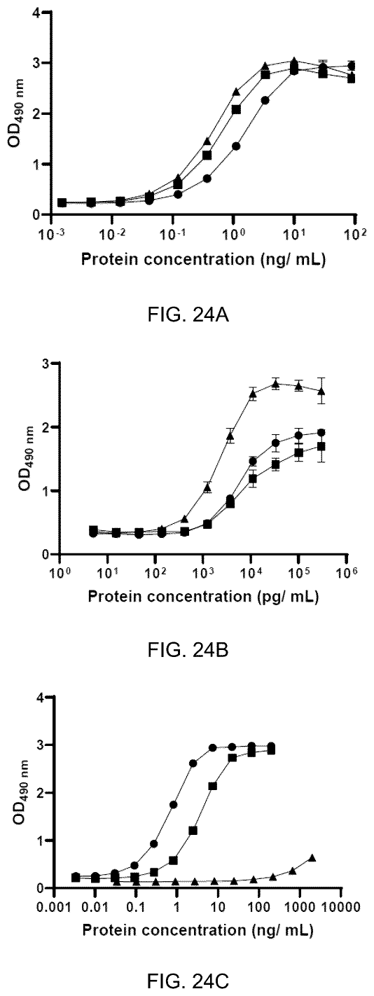

Briefly, for the feline IL-1β-specific ELISA the vaccines and cytokines were tested in a feline IL-1β-specific ELISA to assess their ability to bind to feline IL-1β specific antibodies. ELISA plates were coated overnight with 100 μl/well capture antibody (R&D Systems AF1796) at 500 ng/ml in PBS. After washing, plates were blocked with 1% BSA in PBS. Samples and standards were serial diluted, added to the ELISA plates for 1.5 hours at room temperature and then washed off. The biotinylated anti-fIL-1β detection antibody (R&D Systems BAF1796) was added at a concentration of 400 ng/ml. Plates were incubated for 1 hour at room temperature and washed before adding the streptavidin-HRP (JacksonImmunoResearch, 016-030-084) diluted 1:1000 for 30 minutes. A final washing step was performed and O-phenylenediamine dihydrochloride (OPD, Sigma FAST OPD tablets set, Sigma P9187-50SET) reagent was added. The color reaction was stopped by addition of sulfuric acid and plates read at OD490 nm. Using GraphPad Prism and Microsoft Excel, sample concentrations were plotted against OD490 nm values. A 4-parameter logistic regression model was used for curve fitting and to calculate the EC50 values of each sample i.e. protein concentration resulting in half-maximal OD490 nm value. FIG. 24A shows the recombinant feline IL-1β-D145K mutein produced as described above was positive in a feline IL-1β-specific ELISA.

The binding of the vaccines and cytokines to the IL-1RI, the natural receptor of IL-1β, was tested to confirm that the mutein maintained the conformational structure of the wild type feline IL-1β. As feline IL-1RI was not commercially available, binding of the products to recombinant mouse IL-1RI was assessed instead. Briefly, ELISA plates were coated overnight with 100 μl/well recombinant mouse IL-1RI Fc chimera (R&D 771-MR-100) at 1 μg/ml in carbonate buffer (0.1M NaHCO₃ pH 9.6). After washing, plates were blocked with Superblock (Thermofisher 37515) in PBS. Samples and standards were serial diluted, added to ELISA plates for minimum 1 hour at room temperature before being washed off. The biotinylated anti-fIL-1β detection antibody (R&D Systems BAF1796) was then added at a concentration of 200 ng/ml. Plates were incubated for 1 hour at room temperature and washed before adding the streptavidin-HRP (JacksonImmunoResearch 016-030-084 diluted 1:1000) for 1 hour. A final washing step was performed and OPD reagent (Sigma FAST OPD tablets set, Sigma P9187-50SET) added. The color reaction was stopped with sulfuric acid and plates read at OD450 nm. Using GraphPad Prism and Microsoft Excel, sample concentrations were plotted against OD450 nm values. A 4-parameter logistic regression model was used for curve fitting and to calculate the EC50 values of each sample i.e. protein concentration resulting in half-maximal OD490 nm value. Comparable binding of the feline IL-1β-D145K mutein, purified feline IL-1β wild type of SEQ ID NO:38 (analogously prepared as described herein for IL-1β-D145K mutein; detailed synthesis described in PCT/EP2023/073756), and a commercially available recombinant feline IL-1β protein (R&D 3747-CL-025) to the IL-1RI was observed (FIG. 24B).

The bioactivities of the feline IL-1β-D145K mutein was assessed relative to a commercially available feline IL-1b standard (R&D Systems 1796-FL-010) and purified wild type feline IL-1β using the HEK-Blue IL-1beta reporter cells (InvivoGen hkb-il1bv2). Briefly, HEK-Blue™ IL-1b cells were grown in DMEM Growth medium containing (DMEM with high glucose, 10% HI-FBS, 1× GlutaMAX, 1× antibiotic (Pen/Strep (100 U/ml penicillin, 100 μg/ml streptomycin) or anti-anti (Gibco Antibiotic-Antimycotic, Thermo Fisher Scientific 15240062)) and 100 μg/mL Zeocin (InvivoGen ant-zn-1)). On the day of the assay, cells were detached using 5 mM EDTA in PBS solution, washed with PBS, and then filtered through a 70 μm cell strainer. Cells were resuspended in bioassay medium for a final concentration of 5×10⁴ cells per well of a 96 flat bottom well plate. Cytokine and vaccines were added in a 3-fold dilution series at a starting concentrations of 10 ng/ml and 5 μg/mL respectively. Control wells without cytokines were run on each assay plate. Plates were incubated at 5% CO₂, +37° C. for approximately 20 hours. After 20 hours incubation, cell viability was assessed by visual inspection. 40 μL/well of cell supernatant was added to 160 μL/well QUANTI-Blue Solution (Invivogen rep-qbs). Color development was monitored by measuring OD620 nm 5 hours later.

To determine the bioactivity of the tested samples, sample concentrations were plotted against OD620 nm values. A 4 parameter logistic regression model was used for curve fitting and to calculate the EC50 values of each sample i.e. protein concentration resulting in half-maximal OD620 nm value.

The bioactivity of the mutein feline IL-1β-D145K protein was greatly decreased relative to a commercially available feline IL-1b standard (R&D Systems 1796-FL-010) and purified wild type feline IL-1β; in fact, the bioactivity of the mutein was below the detection limit of the assay (FIG. 24C).

### Example 7

### Coupling of Recombinant Feline IL-1β-D145K Mutein to Modified CMV VLPs

For covalent coupling of recombinantly produced feline IL-1β-D145K mutein to modified CMV VLPs prepared as described above, in particular to CMV-Ntt830-E4 VLPs, recombinant feline IL-1β-D145K mutein was treated with Tris (2-carboxyethyl)phosphin (TCEP) at a 10-fold molar excess of TCEP for 10 minutes at room temperature. Meanwhile, purified CMV-Ntt830-E4 VLPs were diluted to 1.5 mg/ml and reacted with heterobifunctional chemical cross-linker succinimidyl-6-(b-maleimidopropionamide) hexanoate (SMPH) for 1 hour at room temperature. SMPH contains a NHS ester which reacts with the lysine on the surface of the VLP. The amount of SMPH added was 5× molar excess over one VLP coat protein monomer. Cross-linkers which did not react with the VLP were removed by centrifugation using an Amicon-Ultra-15, 100K centrifugal filter (Merck-Millipore, #UFC910024). The SMPH-derivatized VLPs were then washed 4 times with 5 mM Na2HPO4, 2 mM EDTA (pH 7.5).

Next, and briefly, the recombinant feline IL-1β-D145K mutein was added to the VLPs in an about 0.3:1 molar ratio, with respect to the respective chimeric CMV polypeptide monomer, to the previously SMPH derivatized surface charge modified CMV VLPs for 2 hours at RT while shaking. The engineered free cysteine of the feline IL-1β-D145K reacted with the maleimide of the cross-linker SMPH bound to the VLPs to form a stable covalent linkage. The sample was then centrifuged at 10,000 rpm for 10 minutes to remove precipitates and feline IL-1β-D145K proteins that were not associated with the VLPs were removed by gel filtration on a Superdex200 column (run buffer 5 mM NaHPO₄, 2 mM EDTA, pH 7.5). The VLPs in solution were concentrated using a micon-Ultra-15, 100K centrifugal filter device (Merck-Millipore, #UFC910024).

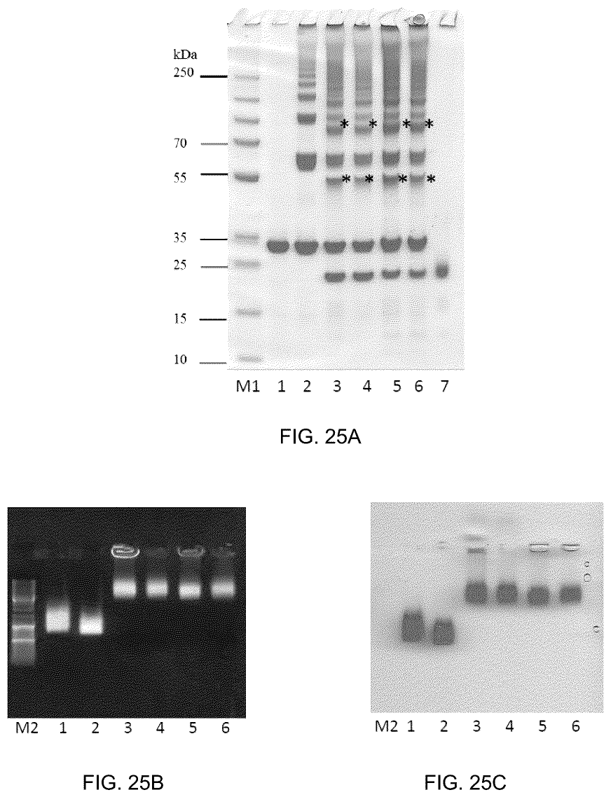

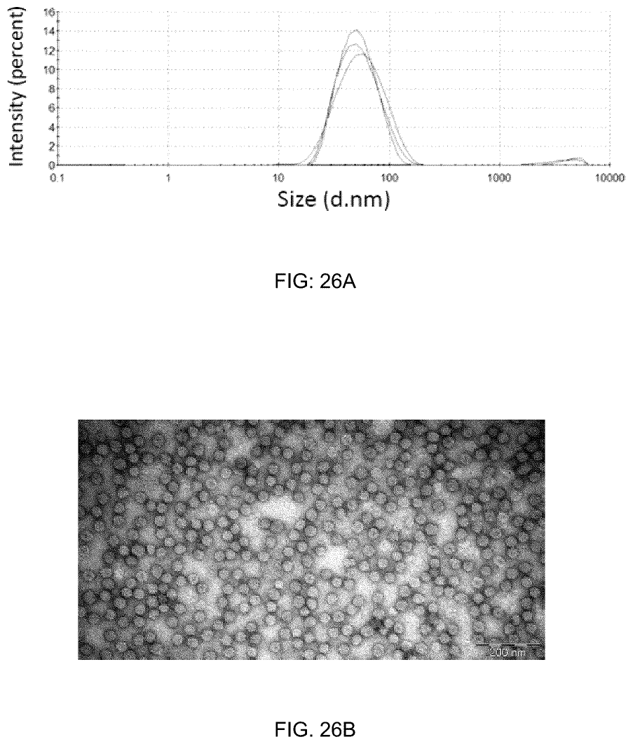

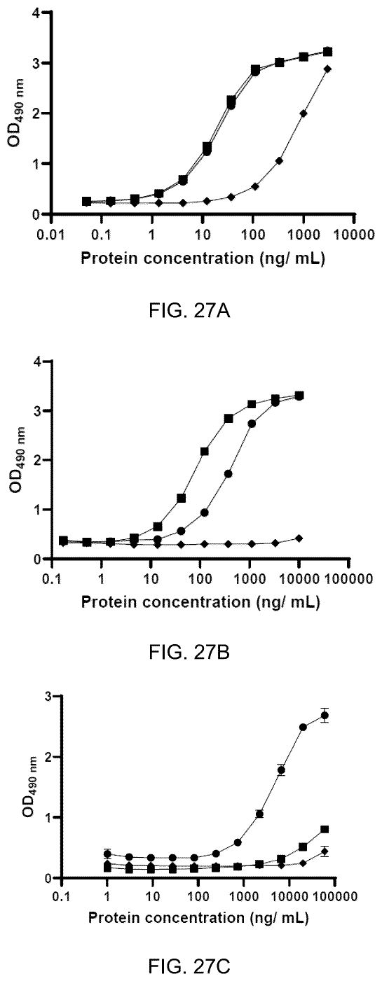

To demonstrate covalent conjugation of feline IL-1β-D145K antigen to VLPs, coupling reactions were analyzed by SDS-PAGE. Prominent conjugation bands were observed following chemical coupling of feline IL-1β-D145K with CMV-Ntt830-E4 VLPs (FIG. 25A). Analysis by native agarose gel electrophoresis showed co-localized bands stained for nucleic acid (FIG. 25B) and protein (FIG. 25C). This is indicative of integral RNA containing VLPs. The shift in migration of the cIL-1b-D145K-CuMVE4 VLPs relative to the CMV-Ntt830-E4 VLPs is due to the attachment of the positively charged feline IL-1β-D145K protein at neutral pH. Analysis by dynamic light scattering (DLS) and electron microscopy (FIG. 26A and FIG. 26B) showed the fIL1b-D145K-CMV-Ntt830-E4 VLP conjugates were of uniform size distribution, not aggregated and were stable in solution and formed typical VLP structures. Bioactivity testing of the fIL-1b-D145K-CMV-Ntt830-E4 vaccine demonstrated that the VLPs are able to bind feline IL-1β-specific antibodies (Fig FIG. 27A) and IL-1RI receptor (FIG. 27B) but do not activate the downstream signalling cascade of the IL-1β (FIG. 27C). This indicates that although the conformational structure of the feline IL-1β-D145K antigen incorporated into the VLPs is authentic, the substitution of the aspartic acid on position 145 with a lysine resulted in the loss of bioactivity of the fIL1b145K-CMV-Ntt830-E4 vaccine.

### Example 8

Induction of Neutralizing Antibodies by Immunizing with the fIL-1b-D145K-CMV-Ntt830-E4 Vaccines

### Immunization of Mice

Five Balb/c mice were immunized twice 21 days apart with 150 μl of fIL-1b-D145K-CMV-Ntt830-E4 VLPs formulated to a concentration of 200 μg/ml in 5 mM NaHPO₄, 2 mM EDTA, pH 7.5. Before each immunization, blood was taken as well as on days 14, 35 and 42 after the first vaccination. Serum was prepared by spinning the blood samples in serum tubes (BD 365968) at 10,000×g for 10 min. Sera were stored at ca. −20° C. until tested.

Measurement of fIL-1β and CMV-VLP Specific IgG Antibodies

Anti-fIL-1β- and CMV-Ntt830-VLP specific IgG antibodies in sera were measured by ELISA. For this purpose, Maxisorp ELISA plates were coated with recombinant feline wild type IL-1β produced as described above or CMV-Ntt830-VLP in 0.1 M Na Carbonate buffer, pH 9.6 at a concentration of 1 μg/ml or 10 μg/ml, respectively, overnight at +4° C. Plates were washed and SuperBlock™ (PBS) Blocking buffer (Thermo Scientific 37518) added for at least 1 hour at room temperature then washed again. Serum samples were pre-diluted 1:100 in 2% BSA in PBS (PBS pH 7.4 (1×) Gibco) with 0.05% Tween 20, transferred to the ELISA plates and subjected to seven 3-fold serial dilutions resulting in total 8 serum dilutions per serum sample. Following incubation for 1.5 hours at room temperature and washing, Horse-radish peroxidase-(HRP-)labelled goat anti-mouse IgG, subclasses 1+2a+2b+3-specific (Jackson ImmunoResearch 115-035-164) diluted 1:2000 in 2% BSA in PBS (PBS pH 7.4 (1×) Gibco) with 0.05% Tween-20 was added. After incubation and washing, OPD substrate (SIGMAFAST™ OPD tablet set Sigma P9187) was used for colorimetric development. The enzymatic reaction was stopped by the addition of 5% H₂SO₄ and the absorbance at 490 nm measured by spectrophotometry using an ELISA reader (Tecan Spark 10). An EC50 titer describes the reciprocal of the dilution, which reaches half of the maximal OD value.

Hek-Blue™ IL-1b Cell Based fIL-1β Neutralization Assay

To determine feline IL-1β neutralizing antibody titers in mouse sera, a HEK-Blue™ IL-1b cell based fIL-1β neutralization assay using the human IL-1b reporter cell line HEK-Blue™ IL-1β Cells (InvivoGen hkb-il1bv2) was performed. Briefly, HEK-Blue IL-1b cells (InvivoGen hkb-il1bv2) were grown and plated at a final concentration of 5×10⁴ cells per well as described above. Sera were heat inactivated for 30 min at +56° C., then diluted 1:12.5 in assay medium (a 4 fold concentrated, lowest sera dilution i.e. 1:50 tested in the assay). A 3-fold serial dilution was prepared for a total of 7 dilutions of each serum, prior to addition of equivalent volume of fIL-1β (R&D Systems 1796-FL-010) at 2000 μg/mL (final concentration in assay is 500 μg/mL). The serum/cytokine solution was incubated for 1 hour at room temperature. 50 μL of the serum/cytokine mixture were then added to the 50 μL of 5×10⁴ HEK-Blue IL-1b cells per well. Positive control wells containing 500 μg/mL of fIL-1β in absence of sera and negative control wells containing cells in assay medium only in absence of sera and fIL-1β were included on each assay plate. Plates were incubated at 5% CO₂, +37° C. for approximately 20 hours. After 20 hours incubation, cell viability was assessed visually. 40 μL/well of cell supernatant was added to 160 μL/well QUANTI-Blue Solution (Invivogen rep-qbs). Color development was monitored by measuring OD620 nm over time. The OD620 nm readout after 5 hours was used to determine the neutralization titers. To determine neutralization titers, results were analyzed using Microsoft Excel and GraphPad prism. Titration curves were generated by plotting the OD620 nm values versus the dilution factor of the serum sample using GraphPad prism software (GraphPad Prism version 8.0.0 for Windows, GraphPad Software, San Diego, California USA). Using a 4-Parameter logistic regression curve fit model with the bottom curve fit value restrained to the average value of the negative controls and the top curve fit value to the mean of the positive control wells included in the assay run the IC50 values, the dilution factor corresponding to half maximum OD values, were determined. Serum fIL-1β neutralization titers of samples at different time points were defined and depicted as the IC50 values of the curve fit.

### Results

### Murine Experiments

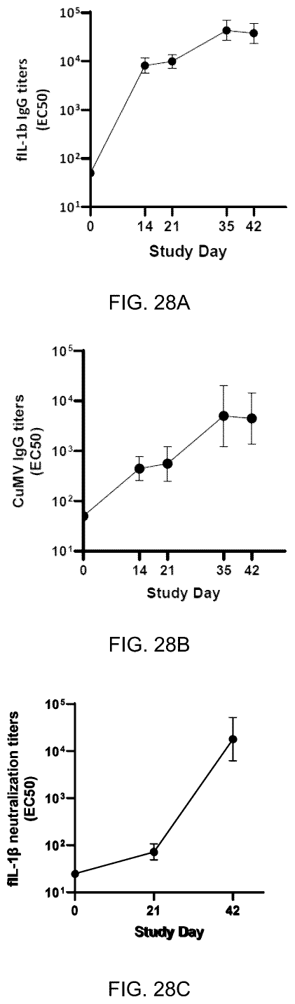

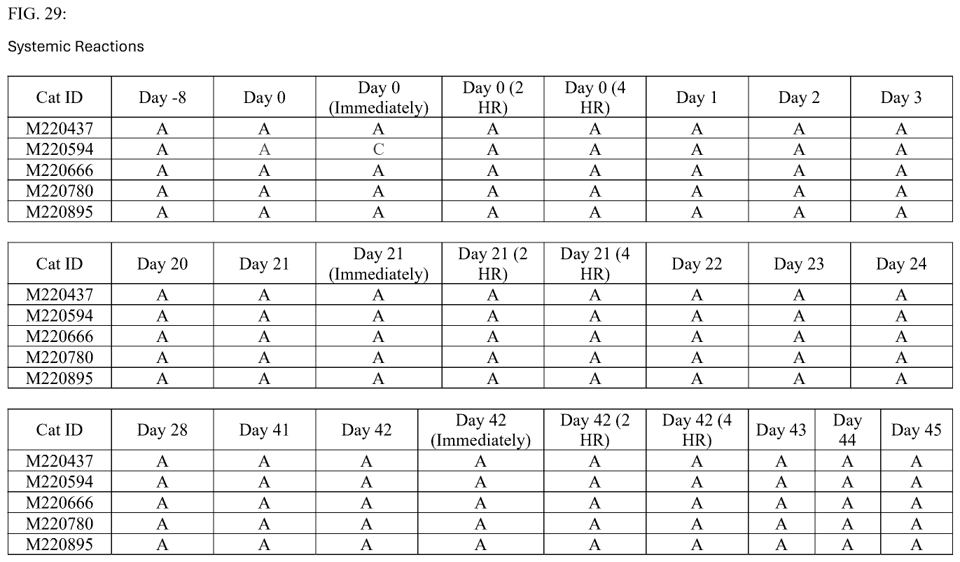

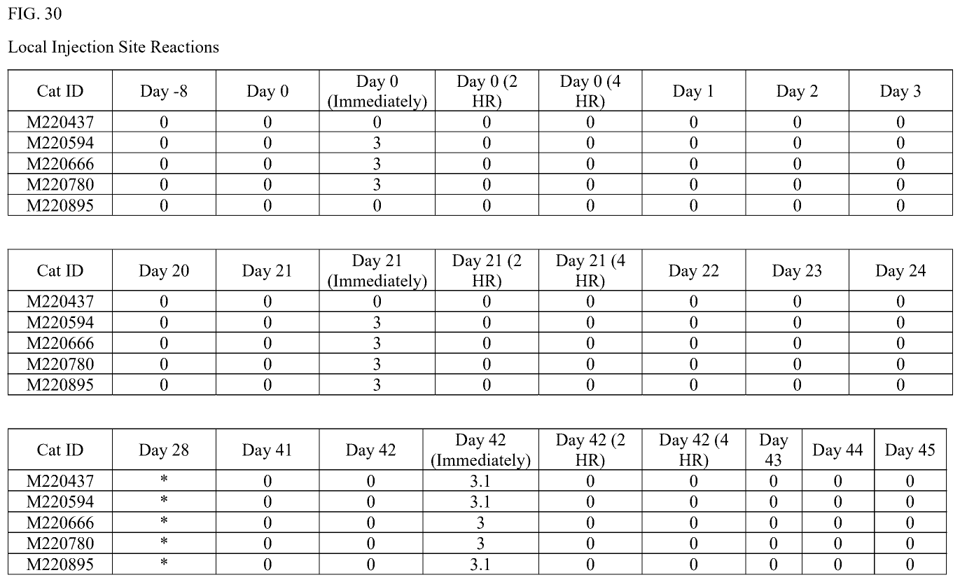

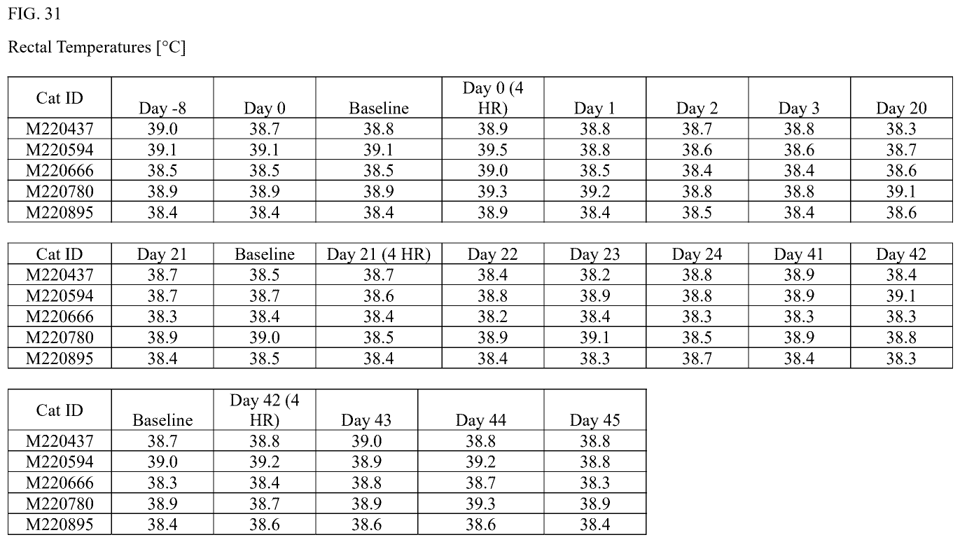

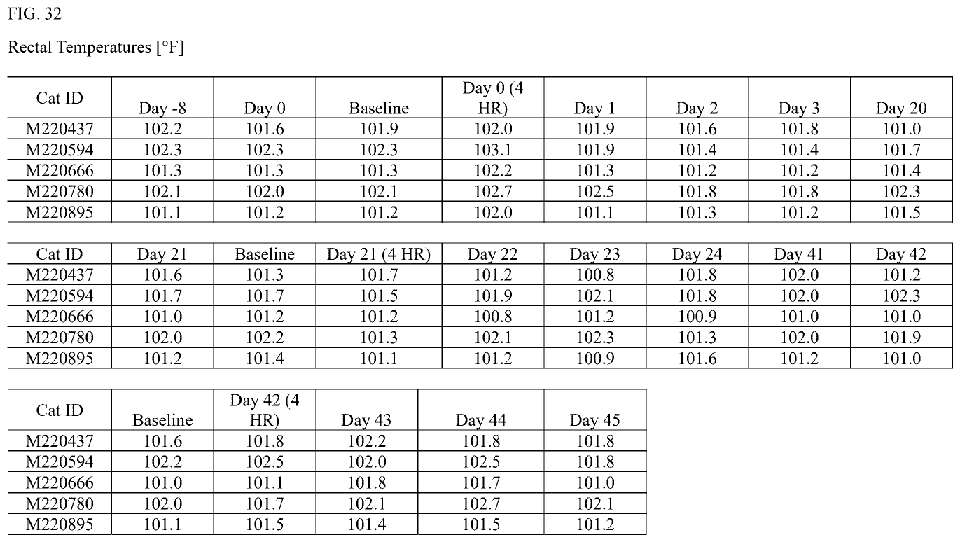

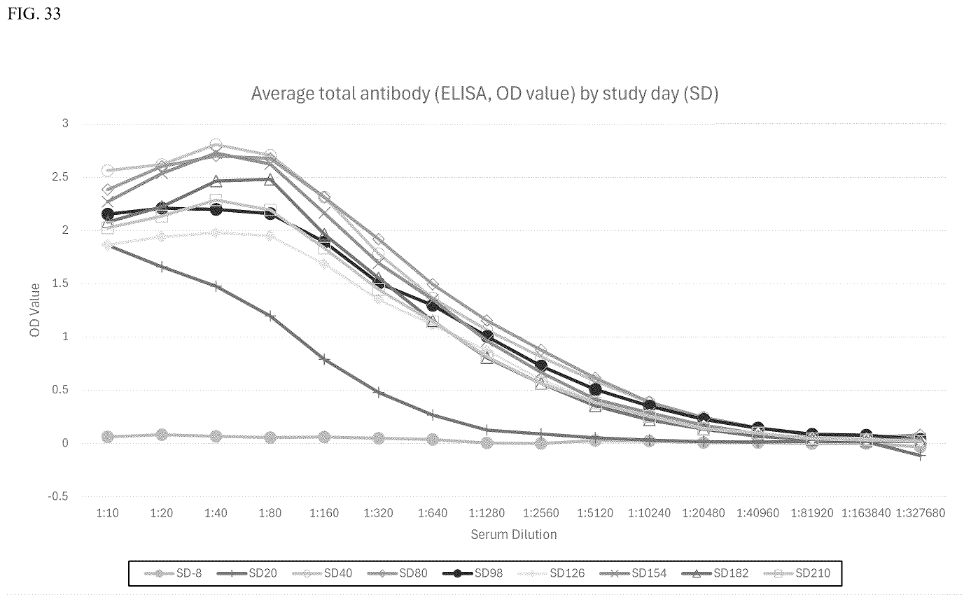

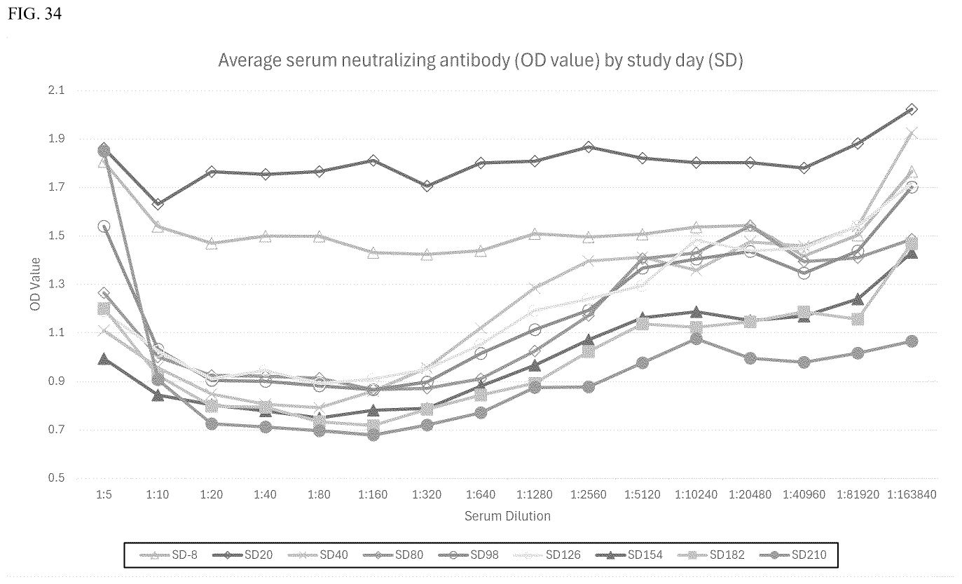

For mice immunized with 30 μg of fIL-1b-D145K-CMV-Ntt830-E4 VLPs, fIL-1β- and VLP carrier-specific IgG antibodies were detected in sera collected from day 14 onwards (FIG. 28A and FIG. 28B). A further increase in the antibody titers was measured in day 35 sera 14 days after administration of the second injection on day 21. Titers remained high until termination of the experiment on day 42.

To test if anti-fIL-1β IgG antibodies induced by immunization with fIL-1b-D145K-CMV-Ntt830-E4 VLPs were neutralizing, they were tested in a HEK-Blue™ IL-1b cell-based fIL-1β neutralization assay, where the reporter construct secretory alkaline phosphatase is expressed under the NF-κB and AP-1 promoter which is activated upon binding of the IL-1β to its receptor. FIG. 28C shows that immune sera collected 21 days and 42 days after first administration of fIL-1b-D145K-CMV-Ntt830-E4 VLPs neutralized fIL-1β, whereas serum collected on day 0 did not.

### Example 9

### Serological and Tolerance Assessment of Feline IL-1β Vaccine in Felines Animals

The animal care and use procedure for this study was reviewed and approved by the Institutional Animal Care and Use Committee (IACUC) of the facility. Animal care and handling was conducted in accordance with the Guide for the Care and Use of Laboratory Animals, 8th Addition, 2011; National Research Council of the National Academies; and the National Academies Press, Washington DC, in conjunction with local standard operating procedures. Minimum space requirements during all phases of the study were met as specified in 9 CFR Subchapter A-Animal Welfare.

Only normal and healthy cats were included in the study. Cats eligible for the study fulfilled the following criteria:
Clinically healthy, no clinically relevant changes at the physical examination and no clinically relevant changes to hematology and clinical chemistry parameters.
No previous treatment with feline IL-1β vaccines.
No treatment with glucocorticoids, immunostimulants, or immune suppressing drugs at least 8 weeks prior to study start.

TABLE A
Animal Information

| | |
|---|---|
| Species: | Felis catus (Feline) |
| Breed: | Domestic short hair |
| Sex: | Male and Female |
| Number of Animals: | 5 |
| Age: | Approximately 5 months on SD 0 |
| Animal Identification: | USDA Ear tattoos and/or microchips |
| Immunological | Conventional |
| Status: | |

Male cats were neutered prior to study initiation and allowed to recover prior to study procedures.

During the course of the study, all animal facilities utilized were shower-in and shower-out per established standard operating procedures. Care order and entry procedures were in place according to established standard operating procedures.

Except when moved to an adjacent pen during husbandry tasks, cats were group housed together in one air space. Cats were acclimated for at least seven days in the room in which they resided during the study.

Cats were housed in a controlled environment that provided temperature, humidity, ventilation, and light cycle management appropriate for their age. Rooms were fitted with self-feeders, automatic waterers, and litter pans with commercial litter. Cats were cared for, and rooms/litter pans cleaned as per established standard operating procedures. Cats were provided with environmental enrichment and socialization with clinical/veterinarian/animal care staff according to the established standard operating procedures.

Cats were provided with access to food and water ad libitum except when anesthetizing for blood draws. Cats were fed a standard commercial dry kibble diet. In addition, treats were provided as deemed necessary.

### Study Design

The Study Design is summarized in Table B.

TABLE B
Schedule of Events

| Study Day | |
|---|---|
| (SD) | Event |
| SD −8 | Injection site observation and record rectal temperatures. Collect one |
| | *SST tube for clinical chemistry and IL-1β serology testing. |
| SD 0 | Injection site observation and record rectal temperatures prior to |
| | vaccination. First vaccination. Immediately, 2 hours, and 4 hours post |
| | vaccination observations. Rectal temperatures at 4 hours (±1 hour) post |
| | vaccination. |
| SD 1, 2, and 3 | Observe for systemic and local site reactions and record rectal |
| | temperatures daily. |
| SD 20 | Injection site observation and record rectal temperatures. Collect one |
| | SST tube for IL-1β serology testing. |
| SD 21 | Injection site observation and record rectal temperatures prior to |
| | vaccination. Second vaccination. Immediately, 2 hours, and 4 hours post |
| | vaccination observations. Rectal temperatures at 4 hours (±1 hour) post |
| | vaccination. |
| SD 22, 23, 24 | Observe for systemic and local site reactions and record rectal |
| | temperatures daily. |
| SD 40 | Injection site observation and record rectal temperatures. Collect one SST |
| | tube for IL-1β serology testing. |
| SD 42 | Injection site observation and record rectal temperatures prior to |
| | vaccination. Third vaccination. Immediately, 2 hours, and 4 hours post |
| | vaccination observations. Rectal temperatures at 4 hours (±1 hour) post |
| | vaccination. |
| SD 43, 44, 45 | Observe for systemic and local site reactions and record rectal |
| | temperatures daily. |
| SD 80 | Collect one SST tube for clinical chemistry and IL-1β serology testing. |
| SD 98 | Collect one SST tube for IL-1β serology testing. |
| SD 126 | Collect one SST tube for IL-1β serology testing. |
| SD 154 | Collect one SST tube for IL-1β serology testing. |
| SD 182 | Collect one SST tube for IL-1β serology testing. |
| SD 210 | Collect one SST tube for clinical chemistry and IL-1β serology testing. |

*SST = Serum Separator Tube.

Cats were chosen randomly from a pool of 40 cats from multiple litters. The randomization was carried out by personnel not involved with performing any clinical or laboratory tasks for the study.

### Vaccine

The following investigational veterinary vaccine (investigational veterinary product (IVP)) was administered to cats during the study:

TABLE C
IVP = “CMV E4 Conjugated Mutein Feline IL-1β”

| Concentration: | 100 | μg/mL |
|---|---|---|
| Volume per Vial: | 7 | mL |
| Shipping Conditions to the Study | Frozen on dry ice | |
| Site: | | |
| Storage Temperature at the Study | ≤−60° | C. |

| | |
|---|---|
| Site: | |
| Administration Location: | Subcutaneous (SQ) |
| Administration Information: | 1.0 mL per dose for 100 |
| | μg at each vaccination |
| Disposition of Remaining IVP: | Freeze at <−60° C. |

The active ingredient in the IVP “CMV E4 Conjugated Mutein Feline IL-1β” is Cucumber Mosaic Virus E4 (CMVE4) virus like particles (VLP) chemically conjugated to feline IL-1β mutein. The feline IL-1β mutein in “CMV E4 Conjugated Mutein Feline IL-1β” is the feline IL-1β-D145K mutein as produced in Example 6. As described in Example 7, the feline IL-1β-D145K mutein was chemically conjugated to CMV-Ntt830-E4 VLPs as described in Example 7, thereby producing “CMV E4 Conjugated Mutein Feline IL-1β”. Production of CMV-Ntt830-E4 VLPs is described in Example 1.

### Vaccine Preparation

On each day of dosing, prior to thawing the IVP formulation, a water bath (with clean water) was pre-equilibrated to 25° C. (+2° C.). The temperature of the water bath was verified and documented.

The IVP formulation was placed in the water bath for no longer than 5 minutes. After complete thawing of the IVP formulation, it was placed on ice until use.

Prior to administration, the IVP formulation was mixed by gentle inversion multiple times; it was not vortexed or centrifuged. The IVP formulation was transported to the animal facility on ice. Once in the animal facility the IVP formulation was equilibrated to room temperature for at least 15 minutes. The IVP formulation was administered within 2 hours of preparation.

The IVP formulation was tested for sterility prior to use.

### Vaccine Administration

Five cats were vaccinated subcutaneously (“SQ”) with 1.0 mL of 100 μg/mL CMV E4 Conjugated Mutein Feline IL-1β on each of Study Day (“SD”) 0, 21, and 42. The first vaccination (SD 0) was administered in the scapular region, right lateral of the midline. The second vaccination (SD 21) was administered in the scapular region, left lateral of the midline. The third vaccination (SD 42) was administered in the scapular region, right lateral of the midline distal to the first vaccination. A new sterile needle was be used for each cat being vaccinated. Vaccine administration was recorded.

### Pre- and Post-Vaccination Observations

Pre- and post-vaccination observations for the cats included localized and systemic reactions. Post-vaccination observations included all observed abnormalities, with focus on the signs listed in Tables D and E.

TABLE D
Localized Reactions
Localized Reaction

| | |
|---|---|
| 0 | No reactions |
| 1 | Pain upon palpation |
| 2 | Injection site markedly warmer than rest of the body |
| 3 | Swelling |
| 4 | Mild Lump <1 cm |
| 5 | Moderate Lump 1-3 cm |
| 6 | Severe Lump >3 cm |
| 7 | Abscess (with drainage) |
| 8 | Scratching/biting/rubbing of injection site |
| 9 | Other (Recorded in comments) |

TABLE E
Systemic Reactions
Systemic Reactions

| | |
|---|---|
| A | Normal |
| B | Generalized Scratching/biting/rubbing |
| C | Vocalization |
| D | Labored breathing |
| E | Lethargy/depression |
| F | Vomiting |
| G | Inappetence |
| H | Diarrhea |
| I | Facial Swelling |
| J | Hives |
| K | Other (Recorded in comments) |

### Systemic and Injection Site Observations

Clinical observations performed on the cats included localized and systemic reactions at the vaccination site.

All cats had the injection site observed, and observations were recorded according to Tables D and E, above. Observations for SDs-8, 0, 1, 2, and 3 were performed on the right. Observations for SDs 20, 21, 22, 23, and 24 were performed on the left. Observations for SDs 41, 42, 43, 44, and 45 were performed on the right.

Initial systemic and injection site reaction observations post-vaccination were conducted immediately, 2 hours, and 4 hours after IVP administration. Reactions were then observed for 3 consecutive days following each vaccination, in accordance with Table B. Systemic and injection site reaction observations were recorded, and are set forth in FIGS. 29 and 30.

### Rectal Temperature Monitoring

All cats had rectal temperatures taken and recorded at approximately the same time of the day to avoid diurnal variation.

For the first vaccination, baseline temperatures were taken on SD-8 and on the day of the first vaccination prior to vaccine administration. For the second and third vaccinations, baseline temperatures were taken the day before each vaccination and the day of each vaccination prior to vaccine administration. The schedule can be seen in Table B.

On vaccination days, temperatures were monitored prior to IVP administration and 4 hours (±1 hour) after IVP administration. Temperatures were then monitored for 3 consecutive days following each vaccination. The schedule can be seen in Table B. Rectal temperature monitoring was recorded, and is set forth in FIGS. 31 and 32. In Celsius, pyrexia is indicated by a temperature >39. %° C. and at least 0.6° C. above baseline. In Fahrenheit, pyrexia is indicated by a temperature >103.5° F. and at least 1.0° F. above baseline.

### Blood Collection

Blood collection volume was based on the following guidelines: up to 15% of the total blood volume may be collected for aggregate collections with no more than 7.5% from any single collection with at least one week rest. These percentages are based on 55 mL of blood per kg body weight. Up to 8.5 mL draw volume tubes were be used. Small cats were weighed, and the blood collection volume was reduced so as not to exceed 7.5% with 1 week rest.

Cats were sedated before blood collections using Ketamine/Butorphanol in accordance with established standard operating procedures. Sedation was be recorded. Blood samples were collected throughout the study as described in Table B. On study days-8, 20, 41, 80, 98, 126, 154, 182, and 210 samples were collected by venipuncture of the jugular, cephalic, or saphenous veins using BD Vacutainer™ SST® serum separation tubes or equivalent. Each blood tube was inverted multiple times to mix the sample well.

Upon receipt in the laboratory the serum was separated by centrifugation and dispensed into labeled 1.0 mL aliquots and/or bulk as dictated by testing needs and stored at −20° C. or colder.

Whole blood was tested on the day of collection by the lab.

### Feline IL-1β Total Antibody ELISA Method

Immunoplates were coated with feline IL-1β coating buffer solution and incubated overnight at 2°−8° C. After overnight incubation, coating solution was removed. Plates were washed a minimum of 3 times with wash buffer. A blocking solution was then added to each plate followed by a room temperature incubation of at least 1 hour with closed/sealed lid.

Feline serum samples were diluted using 2-fold steps until desired dilution was reached. Blocking solution was removed from immunoplate and all feline serum dilutions and controls were added to the plates according to plate template. Sealed plates were incubated at room temperature for at least one hour. After incubation, serum dilutions were removed, and plates were washed a minimum of 3 times with wash buffer. Goat Anti-Feline IgG (H+L)-AP diluted in blocking solution (“conjugate solution”) was then added to the plates. Sealed plates were incubated at room temperature for a minimum of 1 hour.

Conjugate solution was removed, and plates were washed 2 times with wash buffer. Then plates were washed one time with Alkaline Phosphatase buffer. ELISA plates were developed by adding pNPP substrate solution to each well. After addition of substrate, a plate reader was used to perform kinetic monitoring at an OD of 405 nm.

### Feline IL-1β Serum Neutralization Assay Method

CRFK cells were maintained prior to sample testing days. Cells were grown in MEM with 10% heat-inactivated fetal bovine serum. Cells were incubated at 37° C. at 5% CO₂ conditions.

Diluted serum from designated study days was added to the first row in a cell culture microplate while cell media was added to the rest of the plate. Serum was titrated down plate in 2-fold serial dilution steps until desired dilution was reached. After titration, diluted feline IL-1β stimulant was added to the entire plate. Plates underwent preincubation for a minimum of 1 hour up to a maximum of 3 hours. After plate preincubation, cells were added to the whole plate at approximately 1×10⁵ cells/mL. Plates were incubated for approximately 96 hours at 37° C. at 5% CO₂ conditions.

After incubation, culture supernatant from each was transferred to a new microplate. A pNPP substrate solution was added to the entire plate. The reaction was monitored kinetically using a plate reader at an OD of 405 nm.

### Feline IL-1β Total Antibody ELISA Assay Results and Feline IL-1β Serum Neutralization Assay Results

All cats were screened on SD-8 for total feline IL-1β antibodies using an ELISA method prior to being enrolled in the study and were found to be negative for IL-1β antibodies. Cats were vaccinated with a primary vaccination on SD0. All cats had detectable feline IL-1β antibodies by SD20. Antibody titers increased following the two booster vaccinations on SD21 and SD42 with peak antibody titers observed on SD 40, 80, and 154. The results are set forth in FIG. 33.

Prior to being enrolled in the study, all cats were screened on study day (SD)-8 for IL-1β serum neutralization (SN) titers. None of the cats had detectable IL-1β serum neutralization (SN) titers prior to study initiation. After receiving the initial primary vaccination on SD0, all cats remained negative for IL-1β SN titers through SD20. On SD21, all cats received the first booster vaccination, and an IL-1 β SN response was observed in all cats on SD40. The 2nd and final booster vaccination was administered on SD42. On SD80 the IL-1β SN response increased further and the titers remained high through the remainder of the study. The results are set forth in FIG. 34.

Together, these results indicate that vaccination with CMV E4 Conjugated Mutein Feline IL-1β successfully induced serum production of neutralizing anti-IL-1β antibodies in cats. The concentration of total and SN antibodies followed a classic prime-boost pattern, with titers detected following the prime vaccination and increasing after subsequent booster vaccinations.

### Example 10

EXEMPLARY SEQUENCES

| SEQ | |
|---|---|
| ID NO: | Sequence |
| 1 | EEEE |
| 2 | EEEEEEEE |
| 3 | EEEEEEEEEEEE |
| 4 | CGGGSGS |
| 5 | MGQYIKANSKFIGITERRRRPRRGSRSAPSSADANFRVLSQQLSRLNKTL |
| | AAGRPTINHPTFVGSERCKPGYTFTSITLKPPKIDRGSYYGKRLLLPDSV |
| | TEYDKKLVSRIQIRVNPLPKFDSTVWVTVRKVPASSDLSVAAISAMFADG |
| | ASPVLVYQYAASGVQANNKLLYDLSAMRADIGDMRKYAVLVYSKDDALET |
| | DELVLHVDVEHQRIPTSGVLPV |
| 6 | QYIKANSKFIGITE |
| 7 | atgggccagtatattaaggccaactccaaatttatcgggattaccgagcg |
| | tcgacgtcgtccgcgtcgtggttcccgctccgccccctcctccgcggatg |
| | ctaactttagagtcttgtcgcagcagctttcgcgacttaataagacgtta |
| | gcagctggtcgtccaactattaaccacccaacctttgtagggagtgaacg |
| | ctgtaaacctgggtacacgttcacatctatcaccctaaagccaccaaaaa |
| | tagaccgtgggtcttattatggtaaaaggttgttattacctgattcagtc |
| | acggaatatgataagaaacttgtttcgcgcattcaaattcgagttaatcc |
| | tttgccgaaatttgattcaaccgtgtgggtgacagtccgtaaagttcctg |
| | cctcttcggacttatccgttgccgccatttctgctatgtttgcggacgga |
| | gcctcaccggtactggtttatcagtacgctgcatctggagtccaagctaa |
| | caacaaactgttgtatgatctttcggcgatgcgcgctgatataggcgaca |
| | tgagaaagtacgccgtcctcgtgtattcaaaagacgatgcactcgagaca |
| | gacgagttagtacttcatgttgacgtcgagcaccaacgtattcccacatc |
| | tggggtgctcccagtttgataa |
| 8 | GGGS |
| 9 | GGGSGS |
| 10 | MGQYIKANSKFIGITERRRRPRRGSRSAPSSADANFRVLSQQLSRLNKTL |
| | AAGRPTINHPTFVGSERCKPGYTFTSITLKPPKIDRGSGGGSEEEEGGGS |
| | GSYYGKRLLLPDSVTEYDKKLVSRIQIRVNPLPKFDSTVWVTVRKVPASS |
| | DLSVAAISAMFADGASPVLVYQYAASGVQANNKLLYDLSAMRADIGDMRK |
| | YAVLVYSKDDALETDELVLHVDVEHQRIPTSGVLPV |
| 11 | MGQYIKANSKFIGITERRRRPRRGSRSAPSSADANFRVLSQQLSRLNKTL |
| | AAGRPTINHPTFVGSERCKPGYTFTSITLKPPKIDRGSGGGSEEEEEEEE |
| | GGGSGSYYGKRLLLPDSVTEYDKKLVSRIQIRVNPLPKFDSTVWVTVRKV |
| | PASSDLSVAAISAMFADGASPVLVYQYAASGVQANNKLLYDLSAMRADIG |
| | DMRKYAVLVYSKDDALETDELVLHVDVEHQRIPTSGVLPV |
| 12 | MGQYIKANSKFIGITERRRRPRRGSRSAPSSADANFRVLSQQLSRLNKTL |
| | AAGRPTINHPTFVGSERCKPGYTFTSITLKPPKIDRGSGGGSEEEEEEEE |
| | CGGGSGSYYGKRLLLPDSVTEYDKKLVSRIQIRVNPLPKFDSTVWVTVRK |
| | VPASSDLSVAAISAMFADGASPVLVYQYAASGVQANNKLLYDLSAMRADI |
| | GDMRKYAVLVYSKDDALETDELVLHVDVEHQRIPTSGVLPV |
| 13 | MGQYIKANSKFIGITERRRRPRRGSRSAPSSADANFRVLSQQLSRLNKTL |
| | AAGRPTINHPTFVGSERCKPGYTFTSITLKPPKIDRGSGGGSEEEEEEEE |
| | EEEEGGGSGSYYGKRLLLPDSVTEYDKKLVSRIQIRVNPLPKFDSTVWVT |
| | VRKVPASSDLSVAAISAMFADGASPVLVYQYAASGVQANNKLLYDLSAMR |
| | ADIGDMRKYAVLVYSKDDALETDELVLHVDVEHQRIPTSGVLPV |
| 14 | ccatgggccagtatattaaggccaactccaaatttatcgggattaccgag |
| | cgtcgacgtcgtccgcgtcgtggttcccgctccgccccctcctccgcgga |
| | tgctaactttagagtcttgtcgcagcagctttcgcgacttaataagacgt |
| | tagcagctggtcgtccaactattaaccacccaacctttgtagggagtgaa |
| | cgctgtaaacctgggtacacgttcacatctatcaccctaaagccaccaaa |
| | aatagaccgtggatccggtggcggtagcgaagaggaagaagggggtggtt |
| | cgggttcttattatggtaaaaggttgttattacctgattcagtcacggaa |
| | tatgataagaaacttgtttcgcgcattcaaattcgagttaatcctttgcc |
| | gaaatttgattcaaccgtgtgggtgacagtccgtaaagttcctgcctctt |
| | cggacttatccgttgccgccatttctgctatgtttgcggacggagcctca |
| | ccggtactggtttatcagtacgctgcatctggagtccaagctaacaacaa |
| | actgttgtatgatctttcggcgatgcgcgctgatataggcgacatgagaa |
| | agtacgccgtcctcgtgtattcaaaagacgatgcactcgagacagacgag |
| | ttagtacttcatgttgacgtcgagcaccaacgtattcccacatctggggt |
| | gctcccagtttgataagctt |
| 15 | ccatgggccagtatattaaggccaactccaaatttatcgggattaccgag |
| | cgtcgacgtcgtccgcgtcgtggttcccgctccgccccctcctccgcgga |
| | tgctaactttagagtcttgtcgcagcagctttcgcgacttaataagacgt |
| | tagcagctggtcgtccaactattaaccacccaacctttgtagggagtgaa |
| | cgctgtaaacctgggtacacgttcacatctatcaccctaaagccaccaaa |
| | aatagaccgtggatccggtggcggtagcgaggaagaggaagaagaggaag |
| | aagggggtggttcgggttcttattatggtaaaaggttgttattacctgat |
| | tcagtcacggaatatgataagaaacttgtttcgcgcattcaaattcgagt |
| | taatcctttgccgaaatttgattcaaccgtgtgggtgacagtccgtaaag |
| | ttcctgcctcttcggacttatccgttgccgccatttctgctatgtttgcg |
| | gacggagcctcaccggtactggtttatcagtacgctgcatctggagtcca |
| | agctaacaacaaactgttgtatgatctttcggcgatgcgcgctgatatag |
| | gcgacatgagaaagtacgccgtcctcgtgtattcaaaagacgatgcactc |
| | gagacagacgagttagtacttcatgttgacgtcgagcaccaacgtattcc |
| | cacatctggggtgctcccagtttgataagctt |
| 16 | Catatgggccagtatattaaggccaactccaaatttatcgggattaccgag |
| | cgtcgacgtcgtccgcgtcgtggttcccgctccgccccctcctccgcgga |
| | tgctaactttagagtcttgtcgcagcagctttcgcgacttaataagacgt |
| | tagcagctggtcgtccaactattaaccacccaacctttgtagggagtgaa |
| | cgctgtaaacctgggtacacgttcacatctatcaccctaaagccaccaaa |
| | aatagaccgtggatccggtggcggtagcgaagaggaagaagaggaagaag |
| | agtgcgggggtggttcgggttcttattatggtaaaaggttgttattacct |
| | gattcagtcacggaatatgataagaaacttgtttcgcgcattcaaattcg |
| | agttaatcctttgccgaaatttgattcaaccgtgtgggtgacagtccgta |
| | aagttcctgcctcttcggacttatccgttgccgccatttctgctatgttt |
| | gcggacggagcctcaccggtactggtttatcagtacgctgcatctggagt |
| | ccaagctaacaacaaactgttgtatgatctttcggcgatgcgcgctgata |
| | taggcgacatgagaaagtacgccgtcctcgtgtattcaaaagacgatgca |
| | ctcgagacagacgagttagtacttcatgttgacgtcgagcaccaacgtat |
| | tcccacatctggggtgctcccagtttgataagctt |
| 17 | Ccatgggccagtatattaaggccaactccaaatttatcgggattaccgagc |
| | gtcgacgtcgtccgcgtcgtggttcccgctccgccccctcctccgcggat |
| | gctaactttagagtcttgtcgcagcagctttcgcgacttaataagacgtt |
| | agcagctggtcgtccaactattaaccacccaacctttgtagggagtgaac |
| | gctgtaaacctgggtacacgttcacatctatcaccctaaagccaccaaaa |
| | atagaccgtggatccggtggcggtagcgaggaagaagaggaagaagagga |
| | agaagaggaagaagggggtggttcgggttcttattatggtaaaaggttgt |
| | tattacctgattcagtcacggaatatgataagaaacttgtttcgcgcatt |
| | caaattcgagttaatcctttgccgaaatttgattcaaccgtgtgggtgac |
| | agtccgtaaagttcctgcctcttcggacttatccgttgccgccatttctg |
| | ctatgtttgcggacggagcctcaccggtactggtttatcagtacgctgca |
| | tctggagtccaagctaacaacaaactgttgtatgatctttcggcgatgcg |
| | cgctgatataggcgacatgagaaagtacgccgtcctcgtgtattcaaaag |
| | acgatgcactcgagacagacgagttagtacttcatgttgacgtcgagcac |
| | caacgtattcccacatctggggtgctcccagtttgataagctt |
| 18 | tgcgggggtggttcgggttcttattatggtaaaaggttgttattacctga |
| | t |
| 19 | tggatccggtggcggtagcgaagaggaagaagaggaagaagagtgggggg |
| | ggttcgggtt |
| 20 | caaagcttatcaaactgggagcaccccagatgtggga |
| 21 | aggatcgagatctcgatcccgcga |
| 22 | accataataggatccacggtctatttttggtggct |
| 23 | agaccgtggatcctattatggtaaaaggttgttattacct |
| 24 | agtaccggtgaggctccgtccgcaa |
| 25 | ggatccggtggcggtagcgaagaggaagaagggggtggttcgggttctta |
| | t |
| 26 | ggatccggtggcggtagcgaggaagaggaagaagaggaagaagggggtgg |
| | ttcgggttcttat |
| 27 | ggatccggtggcggtagcgaggaagaagaggaagaagaggaagaagagga |
| | agaagggggtggttcgggttcttat |
| 28 | AAMQSVDCKLQDISHKYLVLSNSYELRALHLNGENVNKQVVFHMSFVHGD |
| | ESNNKIPVVLGIKQKNLYLSCVMKDGKPTLQLEKVDPKVYPKRKMEKRFV |
| | FNKIEIKNTVEFESSQYPNWYISTSQVEGMPVFLGNTRGGQDITKFTMEF |
| | SS |
| 29 | AAMQSVDCKLQDISHKYLVLSNSYELRALHLNGENVNKQVVFHMSFVHGD |
| | ESNNKIPVVLGIKQKNLYLSCVMKDGKPTLQLEKVDPKVYPKRKMEKRFV |
| | FNKIEIKNTVEFESSQYPNWYISTSQVEGMPVFLGNTRGGQDITDFTMEF |
| | SS |
| 30 | MGSAAMQSVDCKLQDISHKYLVLSNSYELRALHLNGENVNKQVVFHMSFV |
| | HGDESNNKIPVVLGIKQKNLYLSCVMKDGKPTLQLEKVDPKVYPKRKMEK |
| | RFVFNKIEIKNTVEFESSQYPNWYISTSQVEGMPVFLGNTRGGQDITKFT |
| | MEFSSTSHHHHHHGGCG |
| 31 | HHHHHH |
| 32 | GGCG |
| 33 | Catatgggatccgcagccatgcaatcggtggactgcaagttacaggacata |
| | agccacaaatacctggtgctgtctaactcttatgagcttcgggctctcca |
| | cctcaatggggaaaatgtgaacaaacaagtggtgttccacatgagctttg |
| | tgcacggggatgaaagtaataacaagatacctgtggtcttgggcatcaaa |
| | caaaagaatctgtacctgtcctgtgtgatgaaggatggaaagcccaccct |
| | acagctagagaaggtagaccccaaagtctacccaaagaggaagatggaaa |
| | agcgatttgtcttcaacaagatagaaatcaagaacacagtggaatttgag |
| | tcttctcagtaccctaactggtacatcagcacctctcaagtcgaaggaat |
| | gcctgtcttcctaggaaataccagaggtggccaggatataactaaattca |
| | cgatggaattctcttccactagtcatcatcatcaccatcatggtggttgt |
| | ggataataagcgcttctcgag |
| 34 | tacatatgggatccgcagccatgcaatcggt |
| 35 | atgactagtggaagagaattccatcgtgaatttagttatatcctggcca |
| 36 | HHHHHHGGCG |
| 37 | MGSAAIQSQDYTFRDISQKSLVLSGSYELRALHLNGQNMNQQVVFRMSFV |
| | HGEENSKKIPVVLCIKKNNLYLSCVMKDGKPTLQLEMLDPKVYPKKKMEK |
| | RFVFNKTEIKGNVEFESSQFPNWYISTSQAEEMPVFLGNTKGGQDITDFI |
| | MESASTSHHHHHHGGCG |
| 38 | AAIQSQDYTFRDISQKSLVLSGSYELRALHLNGQNMNQQVVFRMSFVHGE |
| | ENSKKIPVVLCIKKNNLYLSCVMKDGKPTLQLEMLDPKVYPKKKMEKRFV |
| | FNKTEIKGNVEFESSQFPNWYISTSQAEEMPVFLGNTKGGQDITDFIMES |
| | AS |
| 39 | catatgggatccgcagccatacagtcacaggactacacgttccgagacat |
| | aagccaaaagagcctggtgctgtctggctcatacgagcttcgggctctcc |
| | acctcaatggacagaatatgaaccaacaagtggtgttccgcatgagcttt |
| | gtgcacggggaggaaaatagtaagaagataccagtagtgttgtgcatcaa |
| | gaaaaataacctgtacctgtcctgtgtgatgaaagacgggaaacccaccc |
| | tacagctggagatgttagaccccaaagtttacccaaagaagaagatggaa |
| | aagcgatttgtcttcaacaagacagaaatcaagggcaatgtggaatttga |
| | gtcttcccagttccccaactggtacatcagcacctctcaagcagaagaaa |
| | tgcctgtcttcctaggaaataccaaaggtggccaggatataactgacttc |
| | atcatggaaagcgcttccactagtcatcatcatcaccatcatggtggttg |
| | tggataataagcttctcgag |
| 40 | AAIQSQDYTFRDISQKSLVLSGSYELRALHLNGQNMNQQVVFRMSFVHGE |
| | ENSKKIPVVLCIKKNNLYLSCVMKDGKPTLQLEMLDPKVYPKKKMEKRFV |
| | FNKTEIKGNVEFESSQFPNWYISTSQAEEMPVFLGNTKGGQDITKFIMES |
| | AS |
| 41 | GSAAIQSQDYTFRDISQKSLVLSGSYELRALHLNGQNMNQQVVFRMSFVH |
| | GEENSKKIPVVLCIKKNNLYLSCVMKDGKPTLQLEMLDPKVYPKKKMEKR |
| | FVFNKTEIKGNVEFESSQFPNWYISTSQAEEMPVFLGNTKGGQDITKFIM |
| | ESASTSHHHHHHGGCG |
| 42 | ggatccgcagccatacagtcacaggactacacgttccgagacataagcca |
| | aaagagcctggtgctgtctggctcatacgagcttcgggctctccacctca |
| | atggacagaatatgaaccaacaagtggtgttccgcatgagctttgtgcac |
| | ggggaggaaaatagtaagaagataccagtagtgttgtgcatcaagaaaaa |
| | taacctgtacctgtcctgtgtgatgaaagacgggaaacccaccctacagc |
| | tggagatgttagaccccaaagtttacccaaagaagaagatggaaaagcga |
| | tttgtcttcaacaagacagaaatcaagggcaatgtggaatttgagtcttc |
| | ccagttccccaactggtacatcagcacctctcaagcagaagaaatgcctg |
| | tcttcctaggaaataccaaaggtggccaggatataactaaattcatcatg |
| | gaaagcgcttccactagt |
| 43 | atggatccgcagccatacagtcacaggacta |
| 44 | tactagtggaagcgctttccatgatgaatttagttatatcctggccacct |
| | ttggt |
| 45 | MDKSESTSAGRSRRRRPRRGSRSAPSSADANFRVLSQQLSRLNKTLAAGR |
| | PTINHPTFVGSERCKPGYTFTSITLKPPKIDRGSYYGKRLLLPDSVTEYD |
| | KKLVSRIQIRVNPLPKFDSTVWVTVRKVPASSDLSVAAISAMFADGASPV |
| | LVYQYAASGVQANNKLLYDLSAMRADIGDMRKYAVLVYSKDDALETDELV |
| | LHVDVEHQRIPTSGVLPV |
| 46 | DKSESTSAGRSRRRRPRRGSRSAPSSA |
| 47 | QYIKANSKFIGITE |
| 48 | AKFVAAWTLKAAA |
| 49 | GGGSEEEEEEEECGGGSGS |
| 50 | GGGSEEEEGGGSGS |
| 51 | GGGSEEEEEEEEGGGSGS |
| 52 | MAKFVAAWTLKAAARRRRPRRGSRSAPSSADANFRVLSQQLSRLNKTLAA |
| | GRPTINHPTFVGSERCKPGYTFTSITLKPPKIDRGSYYGKRLLLPDSVTE |
| | YDKKLVSRIQIRVNPLPKFDSTVWVTVRKVPASSDLSVAAISAMFADGAS |
| | PVLVYQYAASGVQANNKLLYDLSAMRADIGDMRKYAVLVYSKDDALETDE |
| | LVLHVDVEHQRIPTSGVLPV |
| 53 | PKYVKQNTLKLAT |
| 54 | PHHTALRQAILCWGELMTLA |
| 55 | DIEKKIAKMEKASSVFNVVNS |
| 56 | YSGPLKAEIAQRLEDV |
| 57 | FNNFTVSFWLRVPKVSASHLE |
| 58 | gacgatcgtc |
| 59 | gggggggggggacgatcgtcgggggggggg |
| 60 | MDIPVRSLNCTLRDSQQKSLVMSGPYELKALHLQGQDMEQQVVFSMSFVQ |
| | GEESNDKIPVALGLKEKNLYLSCVLKDDKPTLQLESVDPKNYPKKKMEKR |
| | FVFNKIEINNKLEFESAQFPNWYISTSQAENMPVFLGGTKGGQDITKFTM |
| | QFVSS |
| 61 | MGSAAIQSQDYTFRDISQKSLVLSGSYELRALHLNGQNMNQQVVFRMSFV |
| | HGEENSKKIPVVLCIKKNNLYLSCVMKDGKPTLQLEMLDPKVYPKKKMEK |
| | RFVFNKTEIKGNVEFESSQFPNWYISTSQAEEMPVFLGNTKGGQDITKFI |
| | MESASTSHHHHHHGGCG |

In this specification and the appended claims (“herein”), the singular forms “a,” “an,” and “the” include plural reference unless the context clearly dictates otherwise.

As used herein, the term “and/or” includes any and all combinations of one or more of the associated listed items.

Unless otherwise specified herein, ranges of values set forth herein are intended to operate as a scheme for referring to each separate value falling within the range individually, including but not limited to the endpoints of the ranges, and each separate value of each range set forth herein is hereby incorporated into the specification as if it were individually recited.

As used herein, the terms “about” and “approximately” each mean a range of values that includes the specified value and that one of ordinary skill in the art would reasonably consider to be comparable to the specified value. In embodiments, the terms “about” and “approximately” each mean within a standard deviation using measurements generally acceptable in the art. In embodiments, the terms “about” and “approximately” each mean a range of up to +10%, up to +9%, up to +8%, up to +7%, up to +6%, up to +5%, up to +4%, up to +3%, up to +2%, up to +1%, up to +0.5%, or up to +0.1% of the specified value.

This specification may include references to “embodiment”, “embodiments”, “aspect”, or “aspects”. Each of these words and phrases is not intended to convey a different meaning from the other words and phrases. These words and phrases may refer to the same embodiment or aspect, may refer to different embodiments or aspects, and may refer to more than one embodiment or aspect. The herein described and disclosed embodiments, preferred embodiments and/or very preferred embodiments should apply to all aspects and other embodiments, preferred embodiments and/or very preferred embodiments irrespective of whether is specifically again referred to or irrespective of whether its repetition is avoided for the sake of conciseness. Various embodiments and aspects may be combined in any manner consistent with this disclosure.

The foregoing embodiments and examples are presented by way of example only; the scope of the present disclosure is to be limited only by the claims.

Unless defined otherwise, all technical and scientific terms used herein have the same meaning as commonly understood to one of ordinary skill in the art to which this disclosure belongs.

All references cited in this specification are hereby incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

1. A composition, optionally a veterinary composition, comprising
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, a CMV polypeptide, wherein said CMV polypeptide comprises a coat protein of CMV or an amino acid sequence having a sequence identity of at least 75% with SEQ ID NO:45; and
(b) at least one antigen, wherein said antigen comprises at least one second attachment site, and wherein said antigen is an interleukin 1β D145X mutein (IL-1 β-D145X mutein) antigen; and
wherein (a) and (b) are linked through said at least one first and said at least one second attachment site, optionally via at least one covalent non-peptide bond.

2. A composition, optionally a veterinary composition, comprising
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises,
(i) a CMV polypeptide, wherein said CMV polypeptide comprises a coat protein of CMV or an amino acid sequence having a sequence identity of at least 75% with SEQ ID NO:45; and
(ii) a polypeptide comprising a stretch of consecutive negative amino acids, wherein said negative amino acids are independently selected from aspartic acid and/or glutamic acid, wherein said polypeptide is inserted between any amino acid residue of said CMV polypeptide corresponding to any amino acid residue between position 75 and position 85 of SEQ ID NO:45.
(b) at least one antigen, wherein said antigen comprises at least one second attachment site, and wherein said antigen is an interleukin 1β D145X mutein (IL-1 β-D145X mutein) antigen; and
wherein (a) and (b) are linked through said at least one first and said at least one second attachment site, optionally via at least one covalent non-peptide bond.

3. The composition of claim 1, wherein said chimeric CMV polypeptide further comprises a T helper cell epitope, wherein said T helper cell epitope replaces a N-terminal region of said CMV polypeptide, wherein said N-terminal region of said CMV polypeptide corresponds to amino acids 2-12 of SEQ ID NO:45.

4. The composition of claim 3, wherein said T helper cell epitope comprises the amino acid sequence of SEQ ID NO:47 or SEQ ID NO:48.

5. The composition of CMV of claim 1, wherein said CMV polypeptide is a coat protein of CMV or an amino acid sequence having a sequence identity of at least 90%, optionally 95% with SEQ ID NO:45.

6. The composition of claim 2, wherein said CMV polypeptide comprises the amino acid sequence of SEQ ID NO:5, wherein said polypeptide comprising said stretch of consecutive negative amino acids is inserted between amino acid residues of position 88 and position 89 of said SEQ ID NO:5.

7. The composition of CMV of claim 2, wherein said stretch of consecutive negative amino acids has a length of 3 to 10 amino acids.

8. The composition of CMV of claim 2, wherein said stretch of consecutive negative amino acids consists solely of glutamic acids.

9. The composition of CMV of claim 2, wherein said polypeptide comprising said stretch of consecutive negative amino acids further comprises a first amino acid linker and a second amino acid linker, wherein said first amino acid linker is positioned at the N-terminus of said stretch of consecutive negative amino acids, and said second amino acid linker is positioned at the C-terminus of said stretch of consecutive negative amino acids, and wherein said first and said second amino acid linker is independently selected from the group consisting of:
(a.) a polyglycine linker (G-linker) having an amino acid sequence (Gly) n of a length of n=2-10;
(b.) a glycine-serine linker (GS-linker) comprising at least one glycine and at least one serine, wherein optionally said GS linker has an amino acid sequence of (GS)ᵣ(GₛS)ₜ(GS)ᵤ with r=0 or 1, s=1-5, t=1-5 and u=0 or 1; and
(c.) an amino acid linker (GS*-linker) comprising at least one Gly, at least one Ser, and at least one amino acid selected from Thr, Ala, Lys, and Cys.

10. The composition of claim 1, wherein said polypeptide comprises SEQ ID NO: 49, SEQ ID NO:50 or SEQ ID NO:51.

11. The composition of claim 1, wherein said chimeric CMV polypeptide comprises the amino acid sequence of SEQ ID NO:10, SEQ ID NO:11 or SEQ ID NO:12.

12. The composition of claim 2, wherein said at least one first attachment site is not comprised or part of the polypeptide comprising said stretch of consecutive negative amino acid.

13. The composition of claim 1, wherein said first attachment site is an amino group, optionally an amino group of a lysine residue, and wherein said at least one second attachment site is a sulfhydryl group, optionally a sulfhydryl group of a cysteine residue.

14. The composition of claim 1, wherein said antigen is selected from an interleukin 1β D145K mutein (IL-1β-D145K mutein) antigen.

15. A method of inducing neutralizing antibodies against IL-1β in an animal wherein said method comprising administering a composition of claim 1.

16. (canceled)

17. The composition of claim 2, wherein said chimeric CMV polypeptide further comprises a T helper cell epitope, wherein said T helper cell epitope replaces a N-terminal region of said CMV polypeptide, wherein said N-terminal region of said CMV polypeptide corresponds to amino acids 2-12 of SEQ ID NO:45.

18. The composition of claim 17, wherein optionally said T helper cell epitope comprises the amino acid sequence of SEQ ID NO:47 or SEQ ID NO:48.

19. The composition of CMV of claim 2, wherein said CMV polypeptide is a coat protein of CMV or an amino acid sequence having a sequence identity of at least 90%, optionally 95% with SEQ ID NO:45.

20. The composition of CMV of claim 2, wherein said CMV polypeptide comprises the amino acid sequence of SEQ ID NO:5, wherein said polypeptide comprising said stretch of consecutive negative amino acids is inserted between amino acid residues of position 88 and position 89 of said SEQ ID NO:5.

21. The composition of claim 2, wherein said chimeric CMV polypeptide comprises the amino acid sequence of SEQ ID NO:10, SEQ ID NO:11 or SEQ ID NO:12.


SEQUENCE LISTING (61 sequences)

SEQ ID NO: 1 - protein, 4 aa, synthetic construct
EEEE

SEQ ID NO: 2 - protein, 8 aa, synthetic construct
EEEEEEEE

SEQ ID NO: 3 - protein, 12 aa, synthetic construct
EEEEEEEEEEEE

SEQ ID NO: 4 - protein, 7 aa, synthetic construct
CGGGSGS

SEQ ID NO: 5 - protein, 222 aa, synthetic construct
MGQYIKANSKFIGITERRRRPRRGSRSAPSSADANFRVLSQQLSRLNKTLAAGRPTINHPTFVGSERCKPGYTFTSITLKPPKIDRGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 6 - protein, 14 aa, synthetic construct
QYIKANSKFIGITE

SEQ ID NO: 7 - DNA, 672 bp, synthetic construct
  source 1..672: mol_type = other DNA
atgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtgggtcttattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataa

SEQ ID NO: 8 - protein, 4 aa, synthetic construct
GGGS

SEQ ID NO: 9 - protein, 6 aa, synthetic construct
GGGSGS

SEQ ID NO: 10 - protein, 236 aa, synthetic construct
MGQYIKANSKFIGITERRRRPRRGSRSAPSSADANFRVLSQQLSRLNKTLAAGRPTINHPTFVGSERCKPGYTFTSITLKPPKIDRGSGGGSEEEEGGGSGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 11 - protein, 240 aa, synthetic construct
MGQYIKANSKFIGITERRRRPRRGSRSAPSSADANFRVLSQQLSRLNKTLAAGRPTINHPTFVGSERCKPGYTFTSITLKPPKIDRGSGGGSEEEEEEEEGGGSGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 12 - protein, 241 aa, synthetic construct
MGQYIKANSKFIGITERRRRPRRGSRSAPSSADANFRVLSQQLSRLNKTLAAGRPTINHPTFVGSERCKPGYTFTSITLKPPKIDRGSGGGSEEEEEEEECGGGSGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 13 - protein, 244 aa, synthetic construct
MGQYIKANSKFIGITERRRRPRRGSRSAPSSADANFRVLSQQLSRLNKTLAAGRPTINHPTFVGSERCKPGYTFTSITLKPPKIDRGSGGGSEEEEEEEEEEEEGGGSGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 14 - DNA, 720 bp, synthetic construct
  source 1..720: mol_type = other DNA
ccatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatccggtggcggtagcgaagaggaagaagggggtggttcgggttcttattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagctt

SEQ ID NO: 15 - DNA, 732 bp, synthetic construct
  source 1..732: mol_type = other DNA
ccatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatccggtggcggtagcgaggaagaggaagaagaggaagaagggggtggttcgggttcttattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagctt

SEQ ID NO: 16 - DNA, 736 bp, synthetic construct
  source 1..736: mol_type = other DNA
catatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatccggtggcggtagcgaagaggaagaagaggaagaagagtgcgggggtggttcgggttcttattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagctt

SEQ ID NO: 17 - DNA, 744 bp, synthetic construct
  source 1..744: mol_type = other DNA
ccatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatccggtggcggtagcgaggaagaagaggaagaagaggaagaagaggaagaagggggtggttcgggttcttattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagctt

SEQ ID NO: 18 - DNA, 51 bp, synthetic construct
  source 1..51: mol_type = other DNA
tgcgggggtggttcgggttcttattatggtaaaaggttgttattacctgat

SEQ ID NO: 19 - DNA, 62 bp, synthetic construct
  source 1..62: mol_type = other DNA
tggatccggtggcggtagcgaagaggaagaagaggaagaagagtgcgggggtggttcgggtt

SEQ ID NO: 20 - DNA, 37 bp, synthetic construct
  source 1..37: mol_type = other DNA
caaagcttatcaaactgggagcaccccagatgtggga

SEQ ID NO: 21 - DNA, 24 bp, synthetic construct
  source 1..24: mol_type = other DNA
aggatcgagatctcgatcccgcga

SEQ ID NO: 22 - DNA, 35 bp, synthetic construct
  source 1..35: mol_type = other DNA
accataataggatccacggtctatttttggtggct

SEQ ID NO: 23 - DNA, 40 bp, synthetic construct
  source 1..40: mol_type = other DNA
agaccgtggatcctattatggtaaaaggttgttattacct

SEQ ID NO: 24 - DNA, 25 bp, synthetic construct
  source 1..25: mol_type = other DNA
agtaccggtgaggctccgtccgcaa

SEQ ID NO: 25 - DNA, 51 bp, synthetic construct
  source 1..51: mol_type = other DNA
ggatccggtggcggtagcgaagaggaagaagggggtggttcgggttcttat

SEQ ID NO: 26 - DNA, 63 bp, synthetic construct
  source 1..63: mol_type = other DNA
ggatccggtggcggtagcgaggaagaggaagaagaggaagaagggggtggttcgggttcttat

SEQ ID NO: 27 - DNA, 75 bp, synthetic construct
  source 1..75: mol_type = other DNA
ggatccggtggcggtagcgaggaagaagaggaagaagaggaagaagaggaagaagggggtggttcgggttcttat

SEQ ID NO: 28 - protein, 152 aa, synthetic construct
AAMQSVDCKLQDISHKYLVLSNSYELRALHLNGENVNKQVVFHMSFVHGDESNNKIPVVLGIKQKNLYLSCVMKDGKPTLQLEKVDPKVYPKRKMEKRFVFNKIEIKNTVEFESSQYPNWYISTSQVEGMPVFLGNTRGGQDITKFTMEFSS

SEQ ID NO: 29 - protein, 152 aa, synthetic construct
AAMQSVDCKLQDISHKYLVLSNSYELRALHLNGENVNKQVVFHMSFVHGDESNNKIPVVLGIKQKNLYLSCVMKDGKPTLQLEKVDPKVYPKRKMEKRFVFNKIEIKNTVEFESSQYPNWYISTSQVEGMPVFLGNTRGGQDITDFTMEFSS

SEQ ID NO: 30 - protein, 167 aa, synthetic construct
MGSAAMQSVDCKLQDISHKYLVLSNSYELRALHLNGENVNKQVVFHMSFVHGDESNNKIPVVLGIKQKNLYLSCVMKDGKPTLQLEKVDPKVYPKRKMEKRFVFNKIEIKNTVEFESSQYPNWYISTSQVEGMPVFLGNTRGGQDITKFTMEFSSTSHHHHHHGGCG

SEQ ID NO: 31 - protein, 6 aa, synthetic construct
HHHHHH

SEQ ID NO: 32 - protein, 4 aa, synthetic construct
GGCG

SEQ ID NO: 33 - DNA, 522 bp, synthetic construct
  source 1..522: mol_type = other DNA
catatgggatccgcagccatgcaatcggtggactgcaagttacaggacataagccacaaatacctggtgctgtctaactcttatgagcttcgggctctccacctcaatggggaaaatgtgaacaaacaagtggtgttccacatgagctttgtgcacggggatgaaagtaataacaagatacctgtggtcttgggcatcaaacaaaagaatctgtacctgtcctgtgtgatgaaggatggaaagcccaccctacagctagagaaggtagaccccaaagtctacccaaagaggaagatggaaaagcgatttgtcttcaacaagatagaaatcaagaacacagtggaatttgagtcttctcagtaccctaactggtacatcagcacctctcaagtcgaaggaatgcctgtcttcctaggaaataccagaggtggccaggatataactaaattcacgatggaattctcttccactagtcatcatcatcaccatcatggtggttgtggataataagcgcttctcgag

SEQ ID NO: 34 - DNA, 31 bp, synthetic construct
  source 1..31: mol_type = other DNA
tacatatgggatccgcagccatgcaatcggt

SEQ ID NO: 35 - DNA, 49 bp, synthetic construct
  source 1..49: mol_type = other DNA
atgactagtggaagagaattccatcgtgaatttagttatatcctggcca

SEQ ID NO: 36 - protein, 10 aa, synthetic construct
HHHHHHGGCG

SEQ ID NO: 37 - protein, 167 aa, synthetic construct
MGSAAIQSQDYTFRDISQKSLVLSGSYELRALHLNGQNMNQQVVFRMSFVHGEENSKKIPVVLCIKKNNLYLSCVMKDGKPTLQLEMLDPKVYPKKKMEKRFVFNKTEIKGNVEFESSQFPNWYISTSQAEEMPVFLGNTKGGQDITDFIMESASTSHHHHHHGGCG

SEQ ID NO: 38 - protein, 152 aa, synthetic construct
AAIQSQDYTFRDISQKSLVLSGSYELRALHLNGQNMNQQVVFRMSFVHGEENSKKIPVVLCIKKNNLYLSCVMKDGKPTLQLEMLDPKVYPKKKMEKRFVFNKTEIKGNVEFESSQFPNWYISTSQAEEMPVFLGNTKGGQDITDFIMESAS

SEQ ID NO: 39 - DNA, 520 bp, synthetic construct
  source 1..520: mol_type = other DNA
catatgggatccgcagccatacagtcacaggactacacgttccgagacataagccaaaagagcctggtgctgtctggctcatacgagcttcgggctctccacctcaatggacagaatatgaaccaacaagtggtgttccgcatgagctttgtgcacggggaggaaaatagtaagaagataccagtagtgttgtgcatcaagaaaaataacctgtacctgtcctgtgtgatgaaagacgggaaacccaccctacagctggagatgttagaccccaaagtttacccaaagaagaagatggaaaagcgatttgtcttcaacaagacagaaatcaagggcaatgtggaatttgagtcttcccagttccccaactggtacatcagcacctctcaagcagaagaaatgcctgtcttcctaggaaataccaaaggtggccaggatataactgacttcatcatggaaagcgcttccactagtcatcatcatcaccatcatggtggttgtggataataagcttctcgag

SEQ ID NO: 40 - protein, 152 aa, synthetic construct
AAIQSQDYTFRDISQKSLVLSGSYELRALHLNGQNMNQQVVFRMSFVHGEENSKKIPVVLCIKKNNLYLSCVMKDGKPTLQLEMLDPKVYPKKKMEKRFVFNKTEIKGNVEFESSQFPNWYISTSQAEEMPVFLGNTKGGQDITKFIMESAS

SEQ ID NO: 41 - protein, 166 aa, synthetic construct
GSAAIQSQDYTFRDISQKSLVLSGSYELRALHLNGQNMNQQVVFRMSFVHGEENSKKIPVVLCIKKNNLYLSCVMKDGKPTLQLEMLDPKVYPKKKMEKRFVFNKTEIKGNVEFESSQFPNWYISTSQAEEMPVFLGNTKGGQDITKFIMESASTSHHHHHHGGCG

SEQ ID NO: 42 - DNA, 468 bp, synthetic construct
  source 1..468: mol_type = other DNA
ggatccgcagccatacagtcacaggactacacgttccgagacataagccaaaagagcctggtgctgtctggctcatacgagcttcgggctctccacctcaatggacagaatatgaaccaacaagtggtgttccgcatgagctttgtgcacggggaggaaaatagtaagaagataccagtagtgttgtgcatcaagaaaaataacctgtacctgtcctgtgtgatgaaagacgggaaacccaccctacagctggagatgttagaccccaaagtttacccaaagaagaagatggaaaagcgatttgtcttcaacaagacagaaatcaagggcaatgtggaatttgagtcttcccagttccccaactggtacatcagcacctctcaagcagaagaaatgcctgtcttcctaggaaataccaaaggtggccaggatataactaaattcatcatggaaagcgcttccactagt

SEQ ID NO: 43 - DNA, 31 bp, synthetic construct
  source 1..31: mol_type = other DNA
atggatccgcagccatacagtcacaggacta

SEQ ID NO: 44 - DNA, 55 bp, synthetic construct
  source 1..55: mol_type = other DNA
tactagtggaagcgctttccatgatgaatttagttatatcctggccacctttggt

SEQ ID NO: 45 - protein, 218 aa, synthetic construct
MDKSESTSAGRSRRRRPRRGSRSAPSSADANFRVLSQQLSRLNKTLAAGRPTINHPTFVGSERCKPGYTFTSITLKPPKIDRGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 46 - protein, 27 aa, synthetic construct
DKSESTSAGRSRRRRPRRGSRSAPSSA

SEQ ID NO: 47 - protein, 14 aa, synthetic construct
QYIKANSKFIGITE

SEQ ID NO: 48 - protein, 13 aa, synthetic construct
AKFVAAWTLKAAA

SEQ ID NO: 49 - protein, 19 aa, synthetic construct
GGGSEEEEEEEECGGGSGS

SEQ ID NO: 50 - protein, 14 aa, synthetic construct
GGGSEEEEGGGSGS

SEQ ID NO: 51 - protein, 18 aa, synthetic construct
GGGSEEEEEEEEGGGSGS

SEQ ID NO: 52 - protein, 220 aa, synthetic construct
MAKFVAAWTLKAAARRRRPRRGSRSAPSSADANFRVLSQQLSRLNKTLAAGRPTINHPTFVGSERCKPGYTFTSITLKPPKIDRGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 53 - protein, 13 aa, synthetic construct
PKYVKQNTLKLAT

SEQ ID NO: 54 - protein, 20 aa, synthetic construct
PHHTALRQAILCWGELMTLA

SEQ ID NO: 55 - protein, 21 aa, synthetic construct
DIEKKIAKMEKASSVFNVVNS

SEQ ID NO: 56 - protein, 16 aa, synthetic construct
YSGPLKAEIAQRLEDV

SEQ ID NO: 57 - protein, 21 aa, synthetic construct
FNNFTVSFWLRVPKVSASHLE

SEQ ID NO: 58 - DNA, 10 bp, synthetic construct
  source 1..10: mol_type = other DNA
gacgatcgtc

SEQ ID NO: 59 - DNA, 30 bp, synthetic construct
  source 1..30: mol_type = other DNA
gggggggggggacgatcgtcgggggggggg

SEQ ID NO: 60 - protein, 155 aa, synthetic construct
MDIPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITKFTMQFVSS

SEQ ID NO: 61 - protein, 167 aa, synthetic construct
MGSAAIQSQDYTFRDISQKSLVLSGSYELRALHLNGQNMNQQVVFRMSFVHGEENSKKIPVVLCIKKNNLYLSCVMKDGKPTLQLEMLDPKVYPKKKMEKRFVFNKTEIKGNVEFESSQFPNWYISTSQAEEMPVFLGNTKGGQDITKFIMESASTSHHHHHHGGCG